SEQUENCE LISTING (47 sequences)

SEQ ID NO: 1 - DNA, 1277 bp, Gallus gallus
atggtcgaaatgctgctgttgacaagaattctcttggtgggcttcatctgcgctcttttagtctcctctgggctgacttgtggaccaggcaggggcattggaaaaaggaggcaccccaaaaagctgaccccgttagcctataagcagtttattcccaatgtggcagagaagaccctaggggccagtggaagatatgaagggaagatcacaagaaactccgagagatttaaagaactaaccccaaattacaaccctgacattatttttaaggatgaagagaacacgggagctgacagactgatgactcagcgctgcaaggacaagctgaatgccctggcgatctcggtgatgaaccagtggcccggggtgaagctgcgggtgaccgagggctgggacgaggatggccatcactccgaggaatcgctgcactacgagggtcgcgccgtggacatcaccacgtcggatcgggaccgcagcaagtacggaatgctggcccgcctcgccgtcgaggccggcttcgactgggtctactacgagtccaaggcgcacatccactgctccgtcaaagcagaaaactcagtggcagcgaaatcaggaggctgcttccctggctcagccacagtgcacctggagcatggaggcaccaagctggtgaaggacctgagccctggggaccgcgtgctggctgctgacgcggacggccggctgctctacagtgacttcctcaccttcctcgaccggatggacagctcccgaaagctcttctacgtcatcgagacgcggcagccccgggcccggctgctactgacggcggcccacctgctctttgtggccccccagcacaaccagtcggaggccacagggtccaccagtggccaggcgctcttcgccagcaacgtgaagcctggccaacgtgtctatgtgctgggcgagggcgggcagcagctgctgccggcgtctgtccacagcgtctcattgcgggaggaggcgtccggagcctacgccccactcaccgcccagggcaccatcctcatcaaccgggtgttggcctcctgctacgccgtcatcgaggagcacagttgggcccattgggccttcgcaccattccgcttggctcaggggctgctggccgccctctgcccagatggggccatccctactgccgccaccaccaccactggcatccattggtactcacggctcctctaccgcatcggcagctgggtgctggatggtgacgcgctgcatccgctgggcatggtggcaccggccagctg

SEQ ID NO: 2 - DNA, 1190 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctg

SEQ ID NO: 3 - DNA, 1281 bp, Mus musculus
atgtctcccgcctggctccggccccgactgcggttctgtctgttcctgctgctgctgcttctggtgccggcggcgcggggctgcgggccgggccgggtggtgggcagccgccggaggccgcctcgcaagctcgtgcctcttgcctacaagcagttcagccccaacgtgccggagaagaccctgggcgccagcgggcgctacgaaggcaagatcgcgcgcagctctgagcgcttcaaagagctcacccccaactacaatcccgacatcatcttcaaggacgaggagaacacgggtgccgaccgcctcatgacccagcgctgcaaggaccgtctgaactcactggccatctctgtcatgaaccagtggcctggtgtgaaactgcgggtgaccgaaggccgggatgaagatggccatcactcagaggagtctttacactatgagggccgcgcggtggatatcaccacctcagaccgtgaccgaaataagtatggactgctggcgcgcttagcagtggaggccggcttcgactgggtgtattacgagtccaaggcccacgtgcattgctctgtcaagtctgagcattcggccgctgccaagacaggtggctgctttcctgccggagcccaggtgcgcctagagaacggggagcgtgtggccctgtcagctgtaaagccaggagaccgggtgctggccatgggggaggatgggacccccaccttcagtgatgtgcttattttcctggaccgcgagccaaaccggctgagagctttccaggtcatcgagactcaggatcctccgcgtcggctggcgctcacgcctgcccacctgctcttcattgcggacaatcatacagaaccagcagcccacttccgggccacatttgccagccatgtgcaaccaggccaatatgtgctggtatcaggggtaccaggcctccagcctgctcgggtggcagctgtctccacccacgtggcccttgggtcctatgctcctctcacaaggcatgggacacttgtggtggaggatgtggtggcctcctgctttgcagctgtggctgaccaccatctggctcagttggccttctggcccctgcgactgtttcccagtttggcatggggcagctggaccccaagtgagggtgttcactcctaccctcagatgctctaccgcctggggcgtctcttgctagaagagagcaccttccatccactgggcatgtctggggcaggaagctgaagggactctaaccactgccctcctggaactgctgtgcgtggatcc

SEQ ID NO: 4 - DNA, 1313 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaggctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctg

SEQ ID NO: 5 - DNA, 1256 bp, Brachydanio rerio
atgcggcttttgacgagagtgctgctggtgtctcttctcactctgtccttggtggtgtccggactggcctgcggtcctggcagaggctacggcagaagaagacatccgaagaagctgacacctctcgcctacaagcagttcatacctaatgtcgcggagaagaccttaggggccagcggcagatacgagggcaagataacgcgcaattcggagagatttaaagaacttactccaaattacaatcccgacattatctttaaggatgaggagaacacgggagcggacaggctcatgacacagagatgcaaagacaagctgaactcgctggccatctctgtaatgaaccactggccaggggttaagctgcgtgtgacagagggctgggatgaggacggtcaccattttgaagaatcactccactacgagggaagagctgttgatattaccacctctgaccgagacaagagcaaatacgggacactgtctcgcctagctgtggaggctggatttgactgggtctattacgagtccaaagcccacattcattgctctgtcaaagcagaaaattcggttgctgcgaaatctgggggctgtttcccaggttcggctctggtctcgctccaggacggaggacagaaggccgtgaaggacctgaaccccggagacaaggtgctggcggcagacagcgcgggaaacctggtgttcagcgacttcatcatgttcacagaccgagactccacgacgcgacgtgtgttttacgtcatagaaacgcaagaacccgttgaaaagatcaccctcaccgccgctcacctcctttttgtcctcgacaactcaacggaagatctccacaccatgaccgccgcgtatgccagcagtgtcagagccggacaaaaggtgatggttgttgatgatagcggtcagcttaaatctgtcatcgtgcagcggatatacacggaggagcagcggggctcgttcgcaccagtgactgcacatgggaccattgtggtcgacagaatactggcgtcctgttacgccgtaatagaggaccaggggcttgcgcatttggccttcgcgcccgccaggctctattattacgtgtcatcattcctgtcccccaaaactccagcagtcggtccaatgcgactttacaacaggagggggtccactggtactccaggctcctgtcatcaaatgggaacgtggcttttggacagcaacatgcttcatcctttggggatgtcagtaaactcaagctg

SEQ ID NO: 6 - DNA, 1425 bp, Homo sapiens
  modified_base (1387...1389): n=a, c, g, or t
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagcnnnagccggggggccgggggaggggcgcgggagggggcc

SEQ ID NO: 7 - DNA, 1622 bp, Homo sapiens
catcagcccaccaggagacctcgcccgccgctcccccgggctccccggccatgtctcccgcccggctccggccccgactgcacttctgcctggtcctgttgctgctgctggtggtgcccgcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattccc

SEQ ID NO: 8 - DNA, 1191 bp, Homo sapiens
atggctctcctgaccaatctactgcccttgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgcaaggagagggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctga

SEQ ID NO: 9 - DNA, 1251 bp, Brachydanio rerio
atggacgtaaggctgcatctgaagcaatttgctttactgtgttttatcagcttgcttctgacgccttgtggattagcctgtggtcctggtagaggttatggaaaacgaagacacccaaagaaattaaccccgttggcttacaagcaattcatccccaacgttgctgagaaaacgcttggagccagcggcaaatacgaaggcaaaatcacaaggaattcagagagatttaaagagctgattccgaattataatcccgatatcatctttaaggacgaggaaaacacaaacgctgacaggctgatgaccaagcgctgtaaggacaagttaaattcgttggccatatccgtcatgaaccactggcccggcgtgaaactgcgcgtcactgaaggctgggatgaggatggtcaccatttagaagaatctttgcactatgagggacgggcagtggacatcactacctcagacagggataaaagcaagtatgggatgctatccaggcttgcagtggaggcaggattcgactgggtctattatgaatctaaagcccacatacactgctctgtcaaagcagaaaattcagtggctgctaaatcaggaggatgttttcctgggtctgggacggtgacacttggtgatgggacgaggaaacccatcaaagatcttaaagtgggcgaccgggttttggctgcagacgagaagggaaatgtcttaataagcgactttattatgtttatagaccacgatccgacaacgagaaggcaattcatcgtcatcgagacgtcagaacctttcaccaagctcaccctcactgccgcgcacctagttttcgttggaaactcttcagcagcttcgggtataacagcaacatttgccagcaacgtgaagcctggagatacagttttagtgtgggaagacacatgcgagagcctcaagagcgttacagtgaaaaggatttacactgaggagcacgagggctcttttgcgccagtcaccgcgcacggaaccataatagtggatcaggtgttggcatcgtgctacgcggtcattgagaaccacaaatgggcacattgggcttttgcgccggtcaggttgtgtcacaagctgatgacgtggctttttccggctcgtgaatcaaacgtcaattttcaggaggatggtatccactggtactcaaatatgctgtttcacatcggctcttggctgctggacagagactctttccatccactcgggattttacacttaagttga

SEQ ID NO: 10 - protein, 425 aa, Gallus gallus
MVEMLLLTRILLVGFICALLVSSGLTCGPGRGIGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEHGGTKLVKDLSPGDRVLAADADGRLLYSDFLTFLDRMDSSRKLFYVIETRQPRARLLLTAAHLLFVAPQHNQSEATGSTSGQALFASNVKPGQRVYVLGEGGQQLLPASVHSVSLREEASGAYAPLTAQGTILINRVLASCYAVIEEHSWAHWAFAPFRLAQGLLAALCPDGAIPTAATTTTGIHWYSRLLYRIGSWVLDGDALHPLGMVAPAS

SEQ ID NO: 11 - protein, 396 aa, Mus musculus [sequence omitted]

SEQ ID NO: 12 - protein, 411 aa, Mus musculus
MSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGRDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHSYPQMLYRLGRLLLEESTFHPLGMSGAGS

SEQ ID NO: 13 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVRLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 14 - protein, 418 aa, Brachydanio rerio
MRLLTRVLLVSLLTLSLVVSGLACGPGRGYGRRRHPKKLTPLAYKQFIPNVAEKTLGASGRYEGKITRNSERFKELTPNYNPDIIFKDEENTGADRLMTQRCKDKLNSLAISVMNHWPGVKLRVTEGWDEDGHHFEESLHYEGRAVDITTSDRDKSKYGTLSRLAVEAGFDWVYYESKAHIHCSVKAENSVAAKSGGCFPGSALVSLQDGGQKAVKDLNPGDKVLAADSAGNLVFSDFIMFTDRDSTTRRVFYVIETQEPVEKITLTAAHLLFVLDNSTEDLHTMTAAYASSVRAGQKVMVVDDSGQLKSVIVQRIYTEEQRGSFAPVTAHGTIVVDRILASCYAVIEDQGLAHLAFAPARLYYYVSSFLSPKTPAVGPMRLYNRRGSTGTPGSCHQMGTWLLDSNMLHPLGMSVNSS

SEQ ID NO: 15 - protein, 475 aa, Homo sapiens
  SITE (463): Xaa=unknown amino acid residue
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSSXSRGAGGGAREGA

SEQ ID NO: 16 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 17 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 18 - protein, 416 aa, Brachydanio rerio
MDVRLHLKQFALLCFISLLLTPCGLACGPGRGYGKRRHPKKLTPLAYKQFIPNVAEKTLGASGKYEGKITRNSERFKELIPNYNPDIIFKDEENTNADRLMTKRCKDKLNSLAISVMNHWPGVKLRVTEGWDEDGHHLEESLHYEGRAVDITTSDRDKSKYGMLSRLAVEAGFDWVYYESKAHIHCSVKAENSVAAKSGGCFPGSGTVTLGDGTRKPIKDLKVGDRVLAADEKGNVLISDFIMFIDHDPTTRRQFIVIETSEPFTKLTLTAAHLVFVGNSSAASGITATFASNVKPGDTVLVWEDTCESLKSVTVKRIYTEEHEGSFAPVTAHGTIIVDQVLASCYAVIENHKWAHWAFAPVRLCHKLMTWLFPARESNVNFQEDGIHWYSNMLFHIGSWLLDRDSFHPLGILHLS

SEQ ID NO: 19 - DNA, 1416 bp, Drosophila melanogaster
  CDS (1)..(1413)
atggataaccacagctcagtgccttgggccagtgccgccagtgtcacctgtctctccctgggatgccaaatgccacagttccagttccagttccagctccaaatccgcagcgagctccatctccgcaagcccgcaagaagaacgcaaacgatgcgccacattgcgcatacgcagcgttgcctcagcaggctgacctctctggtggccctgctgctgatcgtcttgccgatggtctttagcccggctcacagctgcggtcctggccgaggattgggtcgtcatagggcgcgcaacctgtatccgctggtcctcaagcagacaattcccaatctatccgagtacacgaacagcgcctccggacctctggagggtgtgatccgtcgggattcgcccaaattcaaggacctcgtgcccaactacaacagggacatccttttccgtgacgaggaaggcaccggagcggatggcttgatgagcaagcgctgcaaggagaagctaaacgtgctggcctactcggtgatgaacgaatggcccggcatccggctgctggtcaccgagagctgggacgaggactaccatcacggccaggagtcgctccactacgagggccgagcggtgaccattgccacctccgatcgcgaccagtccaaatacggcatgctcgctcgcctggccgtcgaggctggattcgattgggtctcctacgtcagcaggcgccacatctactgctccgtcaagtcagattcgtcgatcagttcccacgtgcacggctgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcgcatcgcatcgaggagaagaaccaggtgctcgtacgggatgtggagacgggcgagctgaggccccagcgagtggtcaagttgggcagtgtgcgcagtaagggcgtggtcgcgccgctgacccgcgagggcaccattgtggtcaactcggtggccgccagttgctatgcggtgatcaacagtcagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgtgctgccgcagagctggcgccacgattga

SEQ ID NO: 20 - protein, 471 aa, Drosophila melanogaster
MDNHSSVPWASAASVTCLSLGCQMPQFQFQFQLQIRSELHLRKPARRTQTMRHIAHTQRCLSRLTSLVALLLIVLPMVFSPAHSCGPGRGLGRHRARNLYPLVLKQTIPNLSEYTNSASGPLEGVIRRDSPKFKDLVPNYNRDILFRDEEGTGADGLMSKRCKEKLNVLAYSVMNEWPGIRLLVTESWDEDYHHGQESLHYEGRAVTIATSDRDQSKYGMLARLAVEAGFDWVSYVSRRHIYCSVKSDSSISSHVHGCFTPESTALLESGVRKPLGELSIGDRVLSMTANGQAVYSEVILFMDRNLEQMQNFVQLHTDGGAVLTVTPAHLVSVWQPESQKLTFVFAHRIEEKNQVLVRDVETGELRPQRVVKLGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 21 - protein, 221 aa, Artificial Sequence
  Description of Artificial Sequence Degenerate Shh polypeptide general formula
CGPGRGXGXRRHPKKLTPLAYKQFIPNVAEKTLGASGRYEGKIXRNSERFKELTPNYNPDIIFKDEENTGADRLMTQRCKDKLNXLAISVMNXWPGVXLRVTEGWDEDGHHXEESLHYEGRAVDITTSDRDXSKYGXLXRLAVEAGFDWVYYESKAHIHCSVKAENSVAAKSGGCFPGSAXVXLXXGGXKXVKDLXPGDXVLAADXXGXLXXSDFXXFXDR

SEQ ID NO: 22 - protein, 167 aa, Artificial Sequence
  Description of Artificial Sequence Degenerate hedgehog polypeptide general formula
CGPGRGXXXRRXXXPKXLXPLXYKQFXPXXXEXTLGASGXXEGXXXRXSERFXXLTPNYNPDIIFKDEENXGADRLMTXRCKXXXNXLAISVMNXWPGVXLRVTEGXDEDGHHXXXSLHYEGRAXDITTSDRDXXKYGXLXRLAVEAGFDWVYYESXXHXHXSVKXX

SEQ ID NO: 23 - protein, 175 aa, Homo sapiens
CGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGG

SEQ ID NO: 24 - protein, 174 aa, Homo sapiens
CGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGG

SEQ ID NO: 25 - protein, 176 aa, Homo sapiens
CGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGG

SEQ ID NO: 26 - protein, 175 aa, Artificial Sequence
  Description of Artificial Sequence Consensus sequence
XGPGRXXXXXRRXXXKXLXPLXYKQFXPXVXEKTLGASGRXEGKXXRXSERFKXLXPNYNPDIIFKDEENXGADRLMTXRCKXXXNSLAIXVMNXWPGVKLRVTEGWDEDGHHXXXSLHYEGRAVDITTSDRDRXKYGXLARLAVEAGFDWVYYESXXHXHXSVKXXXXAAXXGG

SEQ ID NO: 27 - DNA, 39 bp, Artificial Sequence
  Mutagenic primer
ggcgatgacgatgacaaattcggaccgggcagggggttc

SEQ ID NO: 28 - DNA, 47 bp, Artificial Sequence
  Mutagenic primer
gcggcgatgacgatgacaaaatcatcggaccgggcagggggttcggg

SEQ ID NO: 29 - DNA, 49 bp, Artificial Sequence
  Mutagenic primer
gagtcatcagcctcccgattttgcgcacaccgagttctctgctttcacc

SEQ ID NO: 30 - DNA, 50 bp, Artificial Sequence
  Mutagenic primer
ccaccaatctcaaagctctcgagctatcagcagcctcccgatttggccgc

SEQ ID NO: 31 - DNA, 31 bp, Artificial Sequence
  Mutagenic primer
ggcccccggcccggaccgcagctctgggctg

SEQ ID NO: 32 - DNA, 52 bp, Artificial Sequence
  Mutagenic primer
gggtaccgggccctcctcgagtcatcagccgcccgcccgcaccgccagtgag

SEQ ID NO: 33 - DNA, 50 bp, Artificial Sequence
  Mutagenic primer
cagcggcgatgacgctgacaaaatcatcggcccgggccgggggccggttg

SEQ ID NO: 34 - DNA, 46 bp, Artificial Sequence
  Mutagenic primer
gtcatcagccgcccgcccgtacgcacagtgagttatcagctttgac

SEQ ID NO: 35 - DNA, 48 bp, Artificial Sequence
  Mutagenic primer
ggcgcctagggtcttctcagccacacaggggataaactgcttgtaggc

SEQ ID NO: 36 - DNA, 41 bp, Artificial Sequence
  Mutagenic primer
gagttccttaaatcgctcggagcacctggagatcttccttc

SEQ ID NO: 37 - DNA, 52 bp, Artificial Sequence
  Mutagenic primer
catccttaaatatgatgtccgggttgcaattgggggtgagttccttaaatcg

SEQ ID NO: 38 - DNA, 48 bp, Artificial Sequence
  Mutagenic primer
catcagcctgtccgctccggtacattcttcatccttaaatatgatgtc

SEQ ID NO: 39 - DNA, 41 bp, Artificial Sequence
  Mutagenic primer
gagatgccaaagcgtttaagcagtccttacacctctgagtc

SEQ ID NO: 40 - DNA, 42 bp, Artificial Sequence
  Mutagenic primer
gtttcactcctggccactgacacatcaccgagatggccaaag

SEQ ID NO: 41 - DNA, 37 bp, Artificial Sequence
  Mutagenic primer
gtagtgcagagactcctcgcagtggtggccatcttcg

SEQ ID NO: 42 - DNA, 39 bp, Artificial Sequence
  Mutagenic primer
ccagcatgccgtacttgcagcgatcgcggtcagacgtgg

SEQ ID NO: 43 - DNA, 38 bp, Artificial Sequence
  Mutagenic primer
cagtggatatgtgccttgcactcgtagtacacccagtc

SEQ ID NO: 44 - DNA, 37 bp, Artificial Sequence
  Mutagenic primer
gtagtgcagagactcgcatgagtggtggccatcttcg

SEQ ID NO: 45 - DNA, 35 bp, Artificial Sequence
  Mutagenic primer
gtacacccagtcgaagcaggcctccaccgccaggc

SEQ ID NO: 46 - DNA, 42 bp, Artificial Sequence
  Mutagenic primer
gagttccttaaatcgctcggagcacctggagatcttcccttc

SEQ ID NO: 47 - DNA, 48 bp, Artificial Sequence; identical to SEQ ID NO: 38
  Mutagenic primer

(19) United States
(12) Patent Application Publication
(10) Pub. No.: US 2004/0038876 A1
(43) Pub. Date: Feb. 26, 2004

(54) Polymer conjugates of hedgehog proteins and uses

(76) Inventors: R. Blake Pepinsky, Arlington, MA (US); Frederick R. Taylor, Milton, MA (US); Ellen A. Garber, Cambridge, MA (US)

Correspondence Address: ROPES & GRAY LLP, ONE INTERNATIONAL PLACE, BOSTON, MA, 02110-2624, US

(21) Appl. No.: 10/244,095

(22) Filed: Sep. 12, 2002

Related U.S. Application Data
(63) Continuation of application No. 09/979,752, now abandoned.
Application No. 09/979,752 is a 371 of international application No. PCT/US00/14741, filed on May 26, 2000.
(60) Provisional application No. 60/137,011, filed on Jun. 1, 1999.
(60) Provisional application No. 60/149,016, filed on Aug. 13, 1999.

(51) Int. Cl. A61K 38/17; C07K 14/47

(52) U.S. Cl. 514/12; 530/350

(57) ABSTRACT

A hedgehog polypeptide comprising hedgehog coupled to a polymer containing a polyalkylene glycol moiety wherein the hedgehog and the polyalkylene glycol moiety are arranged such that the hedgehog has an enhanced bioavailability relative to another hedgehog lacking the polymer and exhibits no decrease in activity as compared to non-conjugated hedgehog. The conjugates of the invention are usefully employed in therapeutic as well as non-therapeutic, e.g., diagnostic, applications.

### BACKGROUND OF THE INVENTION

A peptide family which has been the focus of much research, and efforts to improve its administration and bioavailability, is the hedgehog family of proteins. The hedgehog proteins are a highly conserved family of extracellular signaling proteins with fundamental roles in embryonic development both in vertebrates and in invertebrates (for reviews see Hammerschmidt, M et al., (1997) Trends. Genet. 13: 14-21; Ingham, P. W. (1998) Embo. J., 17: 3505-3511 and Weed, M., et al. (1997) Matrix Biol. 16: 53-58.) The most extensively characterized mammalian hedgehog protein is Sonic hedgehog (Shh), involved in diverse embryonic induction events, including the induction of floor plate and establishment of ventral polarity within the central nervous system as well as proper anterior-posterior patterning of developing limbs (see Riddle, R. D., et al. (1993) Cell 75, 1401-1416; Echelard, Y. et al. (1993) Cell 75, 1417-1471; Roelink, H., et al. (1994) Cell 76, 761-775; and Roelink, H., et al. (1995) Cell 81, 445-455). In mediating these effects, Shh is believed to act both as a short range, contact-dependent inducer and as a long range morphogen (Johnson, R. L. and M. P. Scott (1998), Curr. Opin. Genet. Dev. 8: 450-456).

Shh is synthesized as a 45 kDa precursor protein that is cleaved autocatalytically to yield: (I) a 20 kDa N-terminal fragment (ShhN) that is responsible for all known hedgehog biological activity (SEQ ID NOS. 23-26); and (II) a 25 kDa C-terminal fragment that contains the autoprocessing activity (Lee, J. J., et al. (1994) Science 266, 1528-1536; Bumcrot, D. A., et al. (1995) Mol. Cell Biol. 15, 2294-2303; Porter, J. A., et al. (1995) Nature 374, 363-366). The N-terminal fragment of naturally occurring hedgehog consists of amino acid residues 24-197 of the full-length precursor sequence, of which the N-terminal amino acid residue is a cysteine.

The N-terminal fragment remains membrane-associated through the addition of two lipid tethers: a cholesterol at its C-terminus (Porter, J. A., et al. (1996) Science 274, 255-258; Porter, J. A., et al. (1995) Cell 86, 21-34) and a palmitic acid at its N-terminus (Pepinsky et al., (1998) J. Biol. Chem. 273, 14037-14045). In vivo, the lipid tethers would be critical for restricting the tissue localization of the hedgehog signal and presumably evolved as part of the mechanism for regulating short range-long range signaling. The addition of the cholesterol is catalyzed by the C-terminal domain during the auto-processing step (Porter, J. A., et al. (1996) Cell 86: 21-34), although less is known about the steps leading to palmitylation.

While the mechanism of action of hedgehog proteins is not understood fully, the most recent biochemical and genetic data suggest that the receptor for Shh is the product of the tumor suppressor gene, patched (Marigo, V., et al. (1996) Nature 384, 176-179; Stone, D. M., et al. (1996) Nature 384, 129-134) and that other proteins; smoothened (Alcedo, J., et al. (1996) Cell 86, 221-232), Cubitus interruptus or its mammalian counterpart gli (Dominguez, M., et al. (1996) Science 272, 1621-1625; Alexandre, C., et al. (1996) Genes & Dev. 10, 2003-2013), and fused (Therond, P. P., et al. (1996) Proc. Natl.Acad. Sci. USA 93, 4224-4228) are involved in the hedgehog signaling pathway.

A major factor limiting the usefulness of proteinaceous substances such as hedgehog for their intended application is that, when given parenterally, they are eliminated from the body within a short time. This can occur as a result of metabolism by proteases or by clearance using normal pathways for protein elimination such as by filtration in the kidneys. The oral route of administration of these substances is even more problematic because in addition to proteolysis in the stomach, the high acidity of the stomach may inactivate them before they reach their intended target tissue. The problems associated with these routes of administration of proteins are well known in the pharmaceutical industry, and various strategies are being used in attempts to solve them.

A great deal of work dealing with protein stabilization has been published. One method of stabilization that has been widely used is the addition of an inert polymer to the protein. Numerous ways of conjugating selected amino acid residues of proteins (e.g., cysteines, lysines, N-terminal residues) with polymeric materials are known, including use of dextrans, polyvinyl pyrrolidones, glycopeptides, polyethylene glycol and polyamino acids. The resulting conjugated polypeptides are reported to retain their biological activities and solubility in water for parenteral applications.

### SUMMARY OF THE INVENTION

We have developed a polymer conjugate form of a hedgehog therapeutic with increased bioavailability relative to non-conjugated form and that also has the salutory properties of pegylated proteins in general. We have found that conjugation at the N-terminal cysteine and at the lysine(s) of hedgehog has a deleterious effect on activity of the hedgehog protein. Thus, even if one can show improved bioavailability of a given protein due to conjugation at a given amino acid residue, this is not always predictive of improved efficacy. The ability to produce a product with improved properties is protein dependent and will be governed by specific properties of the test protein.

One aspect of the invention is a conjugated hedgehog protein wherein the hedgehog is covalently bonded to a polymer incorporating as an integral part thereof a polyalkylene glycol at a site other than the lysine(s) and the N-terminal cysteine.

In one particular aspect, the present invention relates to a protein comprising biologically active hedgehog coupled with a polymer comprising a polyalkylene glycol moiety wherein the hedgehog and polyalkylene glycol moiety are arranged such that the biologically active hedgehog therapeutic in the composition has an enhanced half life relative to the hedgehog alone (i.e., in an unconjugated form devoid of the polymer coupled thereto).

Another aspect of the invention is an hedgehog composition comprising biologically active hedgehog coupled with a polymer in which the hedgehog therapeutic is a fusion protein, preferably an immunoglobulin fusion.

In another aspect, the present invention relates to a biologically active hedgehog composition comprising biologically active hedgehog coupled with a polymer comprising a polyalkylene glycol moiety wherein the hedgehog and polyalkylene glycol moiety are arranged such that the biologically active hedgehog in the composition has an enhanced activity relative to hedgehog alone (i.e., in an unconjugated form devoid of the polymer coupled thereto).

The invention further relates to a stable, aqueously soluble, conjugated hedgehog protein comprising a biologically active hedgehog protein covalently coupled to a biologically compatible polyethylene glycol moiety. In such complex, the hedgehog may be covalently coupled to the biologically compatible polyethylene glycol moiety by a labile covalent bond at a free amino acid group of the hedgehog wherein the labile covalent bond is severed in vivo by biochemical hydrolysis and/or proteolysis or through a thiol on a cysteine using a reducible linkage such as a disulfide.

In another aspect, the present invention relates to a dosage form comprising a pharmaceutically acceptable carrier and a stable, aqueously soluble, hedgehog protein comprising hedgehog coupled to a biologically compatible polyethylene glycol.

Modification of a hedgehog protein with a non-toxic polymer may offer certain advantages. Thus, if modifications are made in such a way that the products (polymer-hedgehog protein conjugates) retain all or most of their biological activities, the following properties may result: altered pharmacokinetics and pharmacodynamics leading to increased half-life and alterations in tissue distribution (e.g., ability to stay in the vasculature for longer periods of time), increased stability in solution, reduced immunogenicity, protection from proteolytic digestion and subsequent abolition of activity. Such a formulation is a substantial advance in the pharmaceutical and medical arts and would make a significant contribution to the management of various diseases in which hedgehog has some utility, such as various neuropathies, Parkinson's disease, stroke and inflammatory or autoimmune diseases, and cancers. In particular, the ability to remain for longer periods of time in the vasculature may allow a hedgehog protein of the invention to potentially cross the blood-brain barrier. Further, the thermal stability gained by creating polymer-hedgehog protein conjugates is an advantage when formulating hedgehog protein in powder form for use in subsequent administration.

Hedgehog protein endowed with the improved properties described above may be effective as therapy following either oral, aerosol, or parenteral administration. Other routes of administration, such as nasal and transdermal, may also be possible using the modified hedgehog.

In non-therapeutic (e.g., diagnostic) applications, conjugation of diagnostic and/or reagent species of hedgehog is also contemplated. The resulting conjugated agent is resistant to environmental degradative factors, including solvent- or solution-mediated degradation processes. As a result of such enhanced resistance and increased stability of hedgehog protein, the stability of the active ingredient is able to be significantly increased, with concomitant reliability of the hedgehog protein containing composition in the specific end use for which it is employed.

Other aspects, features, and modifications of the invention will be more fully apparent from the ensuing disclosure and appended claims.

### BRIEF DESCRIPTION OF THE DRAWINGS

FIG. 1: Alignment of the N-terminal domain of Sonic, Indian and Desert Hedgehog proteins (SEQ ID NOS: 23-25, respectively)

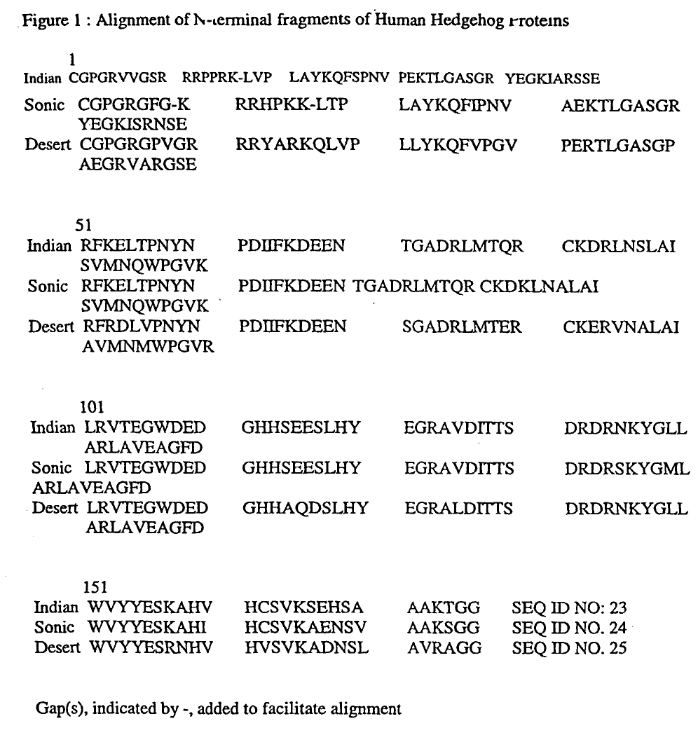

FIG. 2: Purification of a pegylated Shh-polyethylene glycol polymer (“pegylated Shh”) by size exclusion chromatography. Shh II C169 that had been treated with PEG maleimide was subjected to size exclusion chromatography on a Superose 6 FPLC column. Elution fractions were monitored for absorbance at 280 nm and characterized by SDS-PAGE. Peak 1 contains the Shh modified with a single PEG. Peak 2 contains the unmodified Shh.

FIG. 3. Analysis of pegylated Shh by SDS-PAGE. Pegylated Shh was characterized by SDS-PAGE on a 10-20% gradient gel (Daiichi). Proteins were stained with Coomassie brilliant blue. Lane a, prestained high molecular weight markers. Lanes b and d, unmodified Shh II C169. Lanes c and e, pegylated- Shh II C169. Lanes b and c contain 4 μg and lanes d and e, 2 μg, of Shh. Apparent masses of the molecular weight standards are indicated at the left.

FIG. 4. Localization of the site of pegylation by peptide mapping. Pegylated and unmodified Shh II C169 were subjected to peptide mapping analysis. Samples were digested with endoproteinase Lys-C and subjected to reverse phase HPLC on a C₄ column. The column was developed with a 0-70% gradient of acetonitrile in 0.1% trifluoroacetic acid. The column effluent was monitored at 214 nm. Panel a, unmodified Shh II C169. Panel b, pegylated Shh II C169. Arrowheads mark the elution position of the relevant endoproteinase Lys peptide of Shh II C169 containing amino acid resides 164-171.

FIG. 5. Activity of Conjugated and Non-Conjugated Hedgehog. The activity of unmodified Shh II C169 hedgehog or PEGylated Shh II C169 hedgehog at the concentrations indicated on the X axis were assessed in the C3H10T1/2 assay. Following a five day incubation with test compound, the absorbance due to alkaline phosphatase expression which is reflective of hedgehog signaling is shown on the Y axis. The concentration required to achieve 50% response was about 150-200 ng/mL for both the unmodified Shh II C169 and PEGylated Shh II C169.

FIG. 6. Consensus sequence of a hedgehog protein suitable for use in developing the conjugated proteins of the invention. “Xaa” indicates amino acids that differ between the Sonic, Indian and Desert hedgehog proteins. SEQ ID NO: 26.

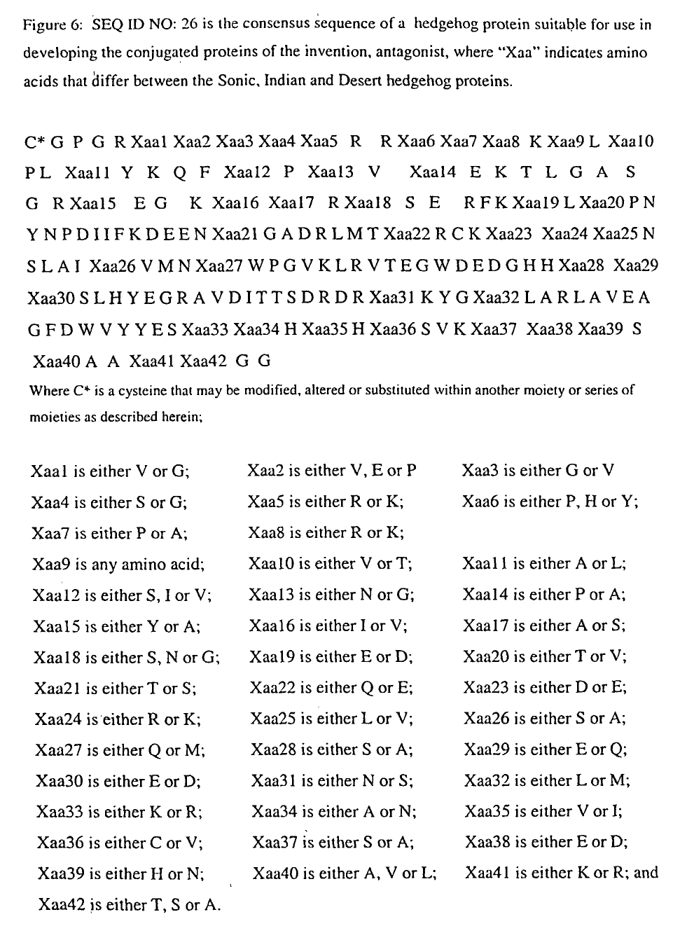

FIG. 7. Analysis of pegylated Shh by SDS-PAGE. Pegylated Shh mutants were characterized by SDS-PAGE on a 10-20% gradient gel (Daiichi) without pegylation (Panel A) after treatment with 5K PEG maleimide (Panel B). Proteins were stained with Coomassie brilliant blue. Lanes are labeled in the figure using numbering based on the gene sequence. A192C corresponds to Shh C1II/A169C, N50C to mutant 17, and N69C, Y80C, N91C, N115C, S177C, K105C, S135C, and S156C to mutants 18-25, respectively. MW stds are prestained high molecular weight markers. Apparent masses of the molecular weight standards are indicated at the left. The −PEG designation in Panel B is Shh C1II/A169A that had not been treated with PEG.

FIG. 8. Structure-activity analysis of the Shh pegylation data. Structure activity data for the pegylated Shh mutants shown in Table I were mapped on the crystal structure of Shh C1II where the positions of the mutated amino acids are indicated.

FIG. 9. Analysis of pegylated Shh by SDS-PAGE. Pegylated Shh mutants were characterized by SDS-PAGE on a 10-20% gradient gel (Daiichi) after treatment with 5K PEG maleimide. Lanes are labeled in the figure. MW stds are prestained high molecular weight markers. Apparent masses of the molecular weight standards are indicated at the left. The −PEG lane designation is Mutant 42 that had not been treated with PEG. The wt designation corresponds to wild type Shh.

### DETAILED DESCRIPTION OF THE INVENTION

Definitions

All references cited in the detailed description are, unless otherwise stipulated, incorporated herein by reference.

I. Definitions

The invention will now be described with reference to the following detailed description of which the following definitions are included:

As used herein, the term hedgehog “antagonist” includes any compound that inhibits hedgehog from binding with its receptor. For the purposes of the invention a hedgehog antagonist also refers to an agent, e.g., a polypeptide such as an anti-hedgehog or anti-patched antibody which can inhibit or block hedgehog and/or patched-mediated binding or which can otherwise modulate hedgehog and/or patched function, e.g., by inhibiting or blocking hedgehog-ligand mediated hedgehog signal transduction. Such an antagonist of the hedgehog/patched interaction is an agent which has one or more of the following properties: (1) it coats, or binds to, a hedgehog on the surface of a hedgehog bearing or secreting cell with sufficient specificity to inhibit a hedgehog-ligand/hedgehog interaction, e.g., the hedgehog/patched interaction; (2) it coats, or binds to, a hedgehog on the surface of a hedgehog- bearing or secreting cell with sufficient specificity to modify, and preferably to inhibit, transduction of a hedgehog-mediated signal e.g., hedgehog/patched-mediated signaling; (3) it coats, or binds to, a hedgehog receptor, (e.g., patched) in or on cells with sufficient specificity to inhibit the hedgehog /patched interaction; (4) it coats, or binds to, a hedgehog receptor (e.g., patched) in or on cells with sufficient specificity to modify, and preferably to inhibit, transduction of hedgehog mediated hedgehog signaling, e.g., patched-mediated hedgehog signaling.

In preferred embodiments the antagonist has one or both of properties 1 and 2. In other preferred embodiments the antagonist has one or both of properties 3 and 4. Moreover, more than one antagonist can be administered to a patient, e.g., an agent which binds to hedgehog can be combined with an agent which binds to patched. As discussed herein, the antagonists used in methods of the invention are not limited to a particular type or structure of molecule so that, for purposes of the invention, any agent capable of binding to hedgehog antigens and which effectively blocks or coats hedgehog is considered to be an equivalent of the antagonists used in the examples herein.

For example, antibodies or antibody homologs (discussed below) as well as other molecules such as soluble forms of the natural binding proteins for hedgehog are useful. Soluble forms of the natural binding proteins for hedgehog include soluble patched peptides, patched fusion proteins, or bifunctional patched/Ig fusion proteins. For example, a soluble form of patched or a fragment thereof may be administered to bind to hedghog, and preferably compete for a hedgehog binding site on cells, thereby leading to effects similar to the administration of antagonists such as anti-hedgehog antibodies. In particular, soluble hedgehog mutants that bind patched but do not elicit hedgehog-dependent signaling are included within the scope of the invention Such hedgehog mutants can act as competitive inhibitors of wild type hedgehog protein and are considered “antagonists”.

The hedgehog antagonists used in the method of the invention bind to, including block or coat, cell-surface hedgehog or patched. These compositions include monoclonal antibody such an anti-hedgehog homolog. Preferred antibodies and homologs for treatment, in particular for human treatment, include human antibody homologs, humanized antibody homologs, chimeric antibody homologs, Fab, Fab′, F(ab′)2 and F(v) antibody fragments, and monomers or dimers of antibody heavy or light chains or mixtures thereof. Thus, monoclonal antibodies against hedgehog are one preferred binding agent in the method of the invention and these are modified by conjugation with polyalkylene polymers as described herein

As used herein, the term “antibody homolog” includes intact antibodies consisting of immunoglobulin light and heavy chains linked via disulfide bonds. The term “antibody homolog” is also intended to encompass a protein comprising one or more polypeptides selected from immunoglobulin light chains, immunoglobulin heavy chains and antigen-binding fragments thereof which are capable of binding to one or more antigens (i.e., hedgehog or patched). The component polypeptides of an antibody homolog composed of more than one polypeptide may optionally be disulfide-bound or otherwise covalently crosslinked. Accordingly, therefore, “antibody homologs” include intact immunoglobulins of types IgA, IgG, IgE, IgD, IgM (as well as subtypes thereof), wherein the light chains of the immunoglobulin may be of types kappa or lambda. “Antibody homologs” also include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like. Thus, antigen-binding fragments, as well as full-length dimeric or trimeric polypeptides derived from the above-described antibodies are themselves useful as well as antobody dimers or multimers created by cross-linking or genetic methods.

As used herein, a “humanized antibody homolog” is an antibody homolog, produced by recombinant DNA technology, in which some or all of the amino acids of a human immunoglobulin light or heavy chain that are not required for antigen binding have been substituted for the corresponding amino acids from a nonhuman mammalian immunoglobulin light or heavy chain. A “human antibody homolog” is an antibody homolog in which all the amino acids of an immunoglobulin light or heavy chain (regardless of whether or not they are required for antigen binding) are derived from a human source.

As used herein, a “chimeric antibody homolog” is an antibody homolog, produced by recombinant DNA technology, in which all or part of the hinge and constant regions of an immunoglobulin light chain, heavy chain, or both, have been substituted for the corresponding regions from another immunoglobulin light chain or heavy chain. In another aspect the invention features a variant of a chimeric molecule which includes: (1) a hedgehog targeting moiety, e.g., a patched moiety capable of binding to hedgehog; (2) optionally, a second peptide, e.g., one which increases solubility or in vivo life time of the hedgehog targeting moiety, e.g., a member of the immunoglobulin super family or fragment or portion thereof, e.g., a portion or a fragment of IgG, e.g., the human IgG1 heavy chain constant region, e.g., CH2, CH3, and hinge regions; and a toxin moiety. The hedgehog targeting moiety can be any naturally occurring hedgehog ligand or fragment thereof, e.g., a patched peptide or a similar conservatively substituted amino acid sequence.

As used herein, the term hedgehog “agonist” includes any compound that activates the hedgehog receptor.

“amino acid”—a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

A hedgehog protein has “biological activity” if it has at least one of the following properties: (i) it has the ability to bind to its receptor, patched or it encodes, upon expression, a polypeptide that has this characteristic; and/or (ii) it may induce alkaline phosphatase activity in C3H10T1/2 cells. The hedgehog protein meeting this functional test of “biological activity” may meet the hedgehog consensus criteria as defined herein (SEQ ID NO: 26) but it may also be a mutant form of hedghog as shown in the Examples. This term includes antagonists and agonists, as defined herein.

The term “bioavailability” refers to the ability of a compound to be absorbed by the body after administration. For instance, a first compound has greater bioavailability than a second compound if, when both are administered subcutaneously in equal amounts, the first compound is absorbed into the blood to a greater extent than the second compound.

As used herein, the term “covalently coupled” means that the specified moieties of the invention (e.g., polyalkylene glycol/hedgehog protein) are either directly covalently bonded to one another, or else are indirectly covalently joined to one another through an intervening moiety or moieties, such as a bridge, spacer, or linkage moiety or moieties. The intervening moiety or moieties are called a “coupling group”. The term “conjugated” is used interchangeably with “covalently coupled”.

“expression control sequence”—a sequence of polynucleotides that controls and regulates expression of genes when operatively linked to those genes.

“expression vector”—a polynucleotide, such as a DNA plasmid or phage (among other common examples) which allows expression of at least one gene when the expression vector is introduced into a host cell. The vector may, or may not, be able to replicate in a cell.

The phrase “extracellular signaling protein” means any protein that is either secreted from a cell, or is associated with the cell membrane, and upon binding to the receptor for that protein on a target cell, triggers a response in the target cell.

An “effective amount” of an agent of the invention is that amount which produces a result or exerts an influence on the particular condition being treated.

“functional equivalent” of an amino acid residue is (i) an amino acid having similar reactive properties as the amino acid residue that was replaced by the functional equivalent; (ii) an amino acid of a ligand of a polypeptide of the invention, the amino acid having similar properties as the amino acid residue that was replaced by the functional equivalent; (iii) a non-amino acid molecule having similar properties as the amino acid residue that was replaced by the functional equivalent.

A first polynucleotide encoding hedgehog protein is “functionally equivalent” compared with a second polynucleotide encoding hedgehog protein if it satisfies at least one of the following conditions:

(a): the “functional equivalent” is a first polynucleotide that hybridizes to the second polynucleotide under standard hybridization conditions and/or is degenerate to the first polynucleotide sequence. Most preferably, it encodes a mutant hedgehog having the activity of an hedgehog protein;

(b) the “functional equivalent” is a first polynucleotide that codes on expression for an amino acid sequence encoded by the second polynucleotide.

The term “hedgehog” includes, but is not limited to, the agents listed herein as well as their functional equivalents. As used herein, the term “functional equivalent” therefore refers to an hedgehog protein or a polynucleotide encoding the hedgehog protein that has the same or an improved beneficial effect on the mammalian recipient as the hedgehog of which it is deemed a functional equivalent. As will be appreciated by one of ordinary skill in the art, a functionally equivalent protein can be produced by recombinant techniques, e.g., by expressing a “functionally equivalent DNA”. Accordingly, the instant invention embraces hedgehog proteins encoded by naturally-occurring DNAs, as well as by non-naturally-occurring DNAs which encode the same protein as encoded by the naturally-occurring DNA. Due to the degeneracy of the nucleotide coding sequences, other polynucleotides may be used to encode hedgehog protein. These include all, or portions of the above sequences which are altered by the substitution of different codons that encode the same amino acid residue within the sequence, thus producing a silent change. Such altered sequences are regarded as equivalents of these sequences. For example, Phe (F) is coded for by two codons, TTC or TTT, Tyr (Y) is coded for by TAC or TAT and His (H) is coded for by CAC or CAT. On the other hand, Trp (W) is coded for by a single codon, TGG. Accordingly, it will be appreciated that for a given DNA sequence encoding a particular hedgehog there will be many DNA degenerate sequences that will code for it. These degenerate DNA sequences are considered within the scope of this invention.

“fusion”—refers to a co-linear linkage of two or more proteins or fragments thereof via their individual peptide backbones through genetic expression of a polynucleotide molecule encoding those proteins. It is preferred that the proteins or fragments thereof be from different sources. Thus, preferred fusion proteins include an hedgehog protein or fragment covalently linked to a second moiety that is not an hedgehog. Specifically, an “hedgehog protein/ Ig fusion” is a protein comprising an hedgehog protein of the invention, or fragment thereof linked to an N-terminus of an immunoglobulin chain wherein a portion of the N-terminus of the immunoglobulin is replaced with the hedgehog protein.

“Heterologous promoter”—as used herein is a promoter which is not naturally associated with a gene or a purified nucleic acid.

“Homology”—as used herein is synonymous with the term “identity” and refers to the sequence similarity between two polypeptides, molecules, or between two nucleic acids. When a position in both of the two compared sequences is occupied by the same base or amino acid monomer subunit (for instance, if a position in each of the two DNA molecules is occupied by adenine, or a position in each of two polypeptides is occupied by a lysine), then the respective molecules are homologous at that position. The percentage homology between two sequences is a function of the number of matching or homologous positions shared by the two sequences divided by the number of positions compared×100. For instance, if 6 of 10 of the positions in two sequences are matched or are homologous, then the two sequences are 60% homologous. By way of example, the DNA sequences CTGACT and CAGGTT share 50% homology (3 of the 6 total positions are matched). Generally, a comparison is made when two sequences are aligned to give maximum homology. Such alignment can be provided using, for instance, the method of Needleman et al., J. Mol Biol. 48: 443-453 (1970), implemented conveniently by computer programs described in more detail below. Homologous sequences share identical or similar amino acid residues, where similar residues are conservative substitutions for, or “allowed point mutations” of, corresponding amino acid residues in an aligned reference sequence. In this regard, a “conservative substitution” of a residue in a reference sequence are those substitutions that are physically or functionally similar to the corresponding reference residues, e.g., that have a similar size, shape, electric charge, chemical properties, including the ability to form covalent or hydrogen bonds, or the like. Particularly preferred conservative substitutions are those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978).

“Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be refered to as homologous at that position. A percentage of homology or identity between sequences is a function of the number of matching or homologous positions shared by the sequences. An. “unrelated” or “non-homologous” sequence shares less than 40 percent identity, though preferably less than 25 percent identity, with an AR sequence of the present invention.

Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

A “hedgehog protein” of the invention is defined in terms of having at least a portion that consists of the consensus amino acid sequence of SEQ ID NO: 26. The term also means a hedgehog polypeptide, or a functional variant of a hedgehog polypeptide, or homolog of a hedgehog polypeptide, or functional variant, which has biological activity.

The term “Hedgehog N-terminal fragment” is used interchangeably with “Hedgehog” and refers to the active mature sequence that is proteolytically cleaved from the hedgehog precursor.

The term “hydrophobic” refers to the tendency of chemical moieties with nonpolar atoms to interact with each other rather than water or other polar atoms. Materials that are “hydrophobic” are, for the most part, insoluble in water. Natural products with hydrophobic properties include lipids, fatty acids, phospholipids, sphingolipids, acylglycerols, waxes, sterols, steroids, terpenes, prostaglandins, thromboxanes, leukotrienes, isoprenoids, retenoids, biotin, and hydrophobic amino acids such as tryptophan, phenylalanine, isoleucine, leucine, valine, methionine, alanine, proline, and tyrosine. A chemical moiety is also hydrophobic or has hydrophobic properties if its physical properties are determined by the presence of nonpolar atoms.

The phrase “internal amino acid” means any amino acid in a peptide sequence that is neither the N-terminal amino acid nor the C-terminal amino acid.

“Isolated” (used interchangeably with “substantially pure”)—when applied to nucleic acid i.e., polynucleotide sequences that encode polypeptides, means an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector, or a portion thereof); or (ii) is linked to a nucleic acid or other chemical moiety other than that to which it is linked in nature; or (iii) does not occur in nature. By “isolated” it is further meant a polynucleotide sequence that is: (i) amplified in vitro by, for example, polymerase chain reaction (PCR); (ii) synthesized chemically; (iii) produce recombinantly by cloning; or (iv) purified, as by cleavage and gel separation.

“Isolated” (used interchangeably with “substantially pure”)—when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature.. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

“multivalent protein complex”—refers to a plurality of proteins (i.e., one or more). A polyalkylene glycol moiety is attached to at least one of the plurality of proteins. The moiety may optionally be in contact with a vesicle. If a protein lacks a poplyalkylene glycol moiety, then that protein may be cross-linked or bind to a protein that does have such a moiety. Each protein may be the same or different and each polyalkylene glycol moiety may be the same or different.

“mutant”—any change in the genetic material of an organism, in particular any change (i.e., deletion, substitution, addition, or alteration) in a wild type polynucleotide sequence or any change in a wild type protein. The term “mutein” is used interchangeably with “mutant”.

“N-terminal end”—refers to the first amino acid residue (amino acid number 1) of the mature form of a protein.

“N-terminal cysteine”—refers to the amino acid number I as shown in SEQ ID NOS. 23-25. In certain embodiments of the hedgehog protein, the N-terminal cysteine has been “modified”. The term “modified” in this regard refers to chemical modification(s) of the N-terminal cysteine such as linkage thereof to another moiety such as a hydrophobic group and/or replacement of the N-terminal cysteine with another moiety, such as a hydrophobic group.

“operatively linked”—a polynucleotide sequence (DNA, RNA) is operatively linked to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“protein”—any polymer consisting essentially of any of the 20 amino acids. Although “polypeptide” is often used in reference to relatively large polypeptides, and “peptide” is often used in reference to small polypeptides, usage of these terms in the art overlaps and is varied. The term “protein” as used herein refers to peptides, proteins and polypeptides, unless otherwise noted.

The terms “peptide(s)”, “protein(s)” and “polypeptide(s)” are used interchangeably herein. The terms “polynucleotide sequence” and “nucleotide sequence” are also used interchangeably herein

A “polymer” is a larger molecule constructed from many smaller structural units called “monomers”, linked together in any conceivable pattern. When only one species of monomer is used to build a larger molecule, the product is called a “homopolymer”, used interchangeably with “polymer”. If the chains are composed of more than one different monomer, the material is generically called a “heteropolymer”. The polymer moiety to which is attached a hedgehog protein or fragment or variant is preferably a polyalkylene glycol polymer but any polymer backbone can be used, most preferably those that are water soluble, non-toxic, and non-immunogenic.

“Recombinant,” as used herein, means that a protein is derived from recombinant, mammalian expression systems. Since hedgehog is not glycosylated nor contains disulfide bonds, it can be expressed in most prokaryotic and eukaryotic expression systems.

“Spacer” sequence refers to a moiety that may be inserted between an amino acid to be modified with a polyalkylene glycol moiety and the remainder of the protein. A spacer is designed to provide separation between the modification and the rest of the protein so as to prevent the modification from interfering with protein function and/or make it easier for the modification to link with a polyalkylene glycol moiety or any other moiety. Thus, if a protein is modified with a polyalkylene glycol polymer at several amino acid sites, there may be two, or more, spacer sequences.

Thus, “substantially pure nucleic acid” is a nucleic acid which is not immediately contiguous with one or both of the coding sequences with which it is normally contiguous in the naturally occurring genome of the organism from which the nucleic acid is derived. Substantially pure DNA also includes a recombinant DNA which is part of a hybrid gene encoding additional hedgehog sequences.

The phrase “surface amino acid” means any amino acid that is exposed to solvent when a protein is folded in its native form.

“standard hybridization conditions”—salt and temperature conditions substantially equivalent to 0.5×SSC to about 5×SSC and 65° C. for both hybridization and wash. The term “standard hybridization conditions” as used herein is therefore an operational definition and encompasses a range of hybridization conditions. Higher stringency conditions may, for example, include hybridizing with plaque screen buffer (0.2% polyvinylpyrrolidone, 0.2% Ficoll 400; 0.2% bovine serum albumin, 50 mM Tris-HCl (pH 7.5); 1 M NaCl; 0.1% sodium pyrophosphate; 1% SDS); 10% dextran sulfate, and 100 μg/ml denatured, sonicated salmon sperm DNA at 65° C. for 12-20 hours, and washing with 75 mM NaCl/7.5 mM sodium citrate (0.5×SSC)/1% SDS at 65° C. Lower stringency conditions may, for example, include hybridizing with plaque screen buffer, 10% dextran sulfate and 110 μg/ml denatured, sonicated salmon sperm DNA at 55° C. for 12-20 hours, and washing with 300 mM NaCl/30 mM sodium citrate (2.0×SSC)/1% SDS at 55° C. See also Current Protocols in Molecular Biology, John Wiley & Sons, Inc. New York, Sections 6.3.1-6.3.6, (1989).

A “therapeutic composition” as used herein is defined as comprising the proteins of the invention and other biologically compatible ingredients. The therapeutic composition may contain excipients such as water, minerals and carriers such as protein.

“wild type”—the naturally-occurring polynucleotide sequence of an exon of a protein, or a portion thereof, or protein sequence, or portion thereof, respectively, as it normally exists in vivo.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. Unless stipulated otherwise, all references cited in the Detailed Description are incorporated herein by reference.

II. General Properties of Isolated Hedgehog Proteins

The various naturally-occurring hedgehog proteins from which the subject proteins can be derived are characterized by a signal peptide, a highly conserved N-terminal region, and a more divergent C-terminal domain. In addition to signal sequence cleavage in the secretory pathway (Lee, J. J. et al. (1992) Cell 71:33-50; Tabata, T. et al. (1992) Genes Dev. 2635-2645; Chang, D. E. et al. (1994) Development 120:3339-3353), hedgehog precursor proteins naturally undergo an internal autoproteolytic cleavage which depends on conserved sequences in the C-terminal portion (Lee et al. (1994) Science 266:1528-1537; Porter et al. (1995) Nature 374:363-366). This autocleavage leads to a 19 kD N-terminal peptide and a C-terminal peptide of 26-28 kD. The N-terminal peptide stays tightly associated with the surface of cells in which it was synthesized, while the C-terminal peptide is freely diffusible both in vitro and in vivo. Cell surface retention of the N-terminal peptide is dependent on autocleavage, as a truncated form of hedgehog encoded by an RNA which terminates precisely at the normal position of internal cleavage is diffusible in vitro (Porter et al. (1995) supra) and in vivo (Porter, J. A. et al. (1996) Cell 86, 21-34). Biochemical studies have shown that the autoproteolytic cleavage of the hedgehog precursor protein proceeds through an internal thioester intermediate, which subsequently is cleaved in a nucleophilic substitution.

The vertebrate family of hedgehog genes includes at least four members, e.g., paralogs of the single drosophila hedgehog gene (reference). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. Isolated hedgehog proteins used in the methods of this invention are naturally occurring or recombinant proteins of the hedgehog family and may be obtainable from either invertebrate or from vertebrate sources (see references below). Members of the vertebrate hedgehog protein family share homology with proteins encoded by the Drosophila hedgehog (hh) gene (Mohler and Vani, (1992) Development 115, 957-971). Other members continue to be identified.

Mouse and chicken Shh and mouse Ihh genes (see, for example, U.S. Pat. No. 5,789,543) encode glycoproteins which undergo cleavage, yielding an amino terminal fragment of about 20 kDa and a carboxy terminal fragment of about 25kDa. The most preferred 20 kDa fragment has the consensus sequence SEQ ID NO: 26 which includes the amino acid sequences of SEQ ID NOS: 23-25. Various other fragments that encompass the 20 kDa moiety are considered within the presently claimed invention. Publications disclosing these sequences, as well as their chemical and physical properties, include Hall et al., (1995) Nature 378, 212-216; Ekker et al., (1995) Current Biology 5, 944-955; Fan et al., (1995) Cell 81, 457-465, Chang et al., (1994) Development 120, 3339-3353; Echelard et al., (1993) Cell 75, 1414-1430 34-38); PCT Patent Application W0 95/23223 (Jessell, Dodd, Roelink and Edlund; PCT Patent Publication W0 95/18856 (Ingham, McMahon and Tabin). U.S. Pat. No. 5,759,811 lists the Genbank accession numbers of a complete mRNA sequence encoding human Sonic hedgehog; a partial sequence of human Indian hedgehog mRNA, 5′ end; and a partial sequence of human Desert hedgehog mRNA. The hedgehog therapeutic compositions of the subject method can be generated by any of a variety of techniques, including purification of naturally occurring proteins, recombinantly produced proteins and synthetic chemistry. Polypeptide forms of the hedgehog therapeutics are preferably derived from vertebrate hedgehog proteins, e.g., have sequences corresponding to naturally occurring hedgehog proteins, or fragments thereof, from vertebrate organisms. However, it will be appreciated that the hedgehog polypeptide can correspond to a hedgehog protein (or fragment thereof) which occurs in any metazoan organism.

The vertebrate family of hedgehog genes includes at least four members, e.g., paralogs of the single drosophila hedgehog gene (SEQ ID No. 19). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. According to the appended sequence listing, (see also Table 1) a chicken Shh polypeptide is encoded by SEQ ID No: 1; a mouse Dhh polypeptide is encoded by SEQ ID No:2; a mouse Ihh polypeptide is encoded by SEQ ID No:3; a mouse Shh polypeptide is encoded by SEQ ID No:4 a zebrafish Shh polypeptide is encoded by SEQ ID No:5; a human Shh polypeptide is encoded by SEQ ID No:6; a human Ihh polypeptide is encoded by SEQ ID No:7; a human Dhh polypeptide is encoded by SEQ ID No. 8; and a zebrafish Thh is encoded by SEQ ID No. 9.

TABLE 1
Guide to hedgehog sequences in Sequence Listing

| | Nucleotide | Amino Acid |
|---|---|---|
| Chicken Shh | SEQ ID No. 1 | SEQ ID No. 10 |
| Mouse Dhh | SEQ ID No. 2 | SEQ ID No. 11 |
| Mouse Ihh | SEQ ID No. 3 | SEQ ID No. 12 |
| Mouse Shh | SEQ ID No. 4 | SEQ ID No. 13 |
| Zebrafish Shh | SEQ ID No. 5 | SEQ ID No. 14 |
| Human Shh | SEQ ID No. 6 | SEQ ID No. 15 |
| Human Ihh | SEQ ID No. 7 | SEQ ID No. 16 |
| Human Dhh | SEQ ID No. 8 | SEQ ID No. 17 |
| Zebrafish Thh | SEQ ID No. 9 | SEQ ID No. 18 |
| Drosophila HH | SEQ ID No. 19 | SEQ ID No. 20 |

In addition to the sequence variation between the various hedgehog homologs, the hedgehog proteins are apparently present naturally in a number of different forms, including a pro-form, a full-length mature form, and several processed fragments thereof. The pro-form includes an N-terminal signal peptide for directed secretion of the extracellular domain, while the full-length mature form lacks this signal sequence.

As described above, further processing of the mature form occurs in some instances to yield biologically active fragments of the protein. For instance, sonic hedgehog undergoes additional proteolytic processing to yield two peptides of approximately 19 kDa and 27 kDa, the 19 kDa fragment corresponding to an proteolytic N-terminal portion of the mature protein.

In addition to the sequence variation between the various hedgehog homologs, the proteins are apparently present naturally in a number of different forms, including a pro-form, a full-length mature form, and several processed fragments thereof. The pro-form includes an N-terminal signal peptide for directed secretion of the extracellular domain, while the full-length mature form lacks this signal sequence.

Family members useful in the methods of the invention include any of the naturally-occurring native hedgehog proteins including allelic, phylogenetic counterparts or other variants thereof, whether naturally-sourced or produced chemically including muteins or mutant proteins, as well as recombinant forms and new, active members of the hedgehog family. Particularly useful hedgehog polypeptides have portions that include all or part of SEQ ID NOS: 23-26.

Isolated hedgehog polypeptides used in the method of the invention have biological activity. The polypeptides include an amino acid sequence at least 60%, 80%, 90%, 95%, 98%, or 99% homologous to an amino acid sequence from SEQ ID NOS; 23-26. The polypeptide can also include an amino acid sequence essentially the same as an amino acid sequence in SEQ ID NOS: 21-24. The polypeptide is at least 5, 10, 20, 50, 100, or 150 amino acids in length and includes at least 5, preferably at least 10, more preferably at least 20, most preferably at least 50, 100, or 150 contiguous amino acids from SEQ ID NOS: 23-26.

Polypeptides of the invention include those which arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

In one embodiment, isolated hedgehog is a hedgehog polypeptide with one or more of the following characteristics:

(i) it has at least 30, 40, 42, 50, 60, 70, 80, 90 or 95% sequence identity with amino acids of SEQ ID NOS: 23-26;

(ii) it has a cysteine or a functional equivalent as the N-terminal end;

(iii) it may induce alkaline phosphatase activity in C3H10T1/2 cells;

(iv) it has an overall sequence identity of at least 50%, preferably at least 60%, more preferably at least 70, 80, 90, or 95%, with a polypeptide of SEQ ID NO; 23-26

(v) it can be isolated from natural sources such as mammalian cells;

(vi) it can bind or interact with patched; and

(vii) it is modified at at least one amino acid residue by a polyalkylene glycol polymer attached to the residue or, optionally, via a linker molecule to the amino acid residue.

Preferred nucleic acids encode a polypeptide comprising an amino acid sequence at least 60% homologous or identical, more preferably 70% homologous or identical, and most preferably 80% homologous or identical with an amino acid sequence selected from the group consisting of SEQ ID Nos: 23-26. Nucleic acids which encode polypeptides at least about 90%, more preferably at least about 95%, and most preferably at least about 98-99% homology or identity with an amino acid sequence represented in one of SEQ ID Nos:23-26 are also within the scope of the invention.

In another embodiment, the hedgehog protein is a polypeptide encodable by a nucleotide sequence that hybridizes under stringent conditions to a hedgehog coding sequence represented in one or more of SEQ ID NOS:1-9 or 19. Appropriate stringency conditions which promote DNA hybridization, for example, 6.0×sodium chloride/sodium citrate (SSC) at about 45 degrees C., followed by a wash of 2.0×SSC at 50 degrees C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50 degrees C. to a high stringency of about 0.2×SSC at 50 degrees C. In addition, the temperature in the wash step can be increased from low stringency conditions at room temperature, about 22 degrees C., to high stringency conditions at about 65 degrees C.

Preferred nucleic acids encode a hedgehog polypeptide comprising an amino acid sequence at least 60% homologous, more preferably 70% homologous and most preferably 80% homologous with an amino acid sequence selected from the group consisting of SEQ ID Nos: 8-14. Nucleic acids which encode polypeptides at least about 90%, more preferably at least about 95%, and most preferably at least about 98-99% homology with an amino acid sequence represented in one of SEQ ID Nos: 10-18 or 20 are also within the scope of the invention.

Hedgehog polypeptides preferred by the present invention, in addition to native hedgehog proteins, are at least 60% homologous, more preferably 70% homologous and most preferably 80% homologous with an amino acid sequence represented by any of SEQ ID Nos:10-18 or 20. Polypeptides which are at least 90%, more preferably at least 95%, and most preferably at least about 98-99% homologous with a sequence selected from the group consisting of SEQ ID Nos: 10-18 or 20 are also within the scope of the invention.

With respect to fragments of hedgehog polypeptide, preferred hedgehogs moieties include at least 50 amino acid residues of a hedgehog polypeptide, more preferably at least 100, and even more preferably at least 150.

Another preferred hedgehog polypeptide which can be included in the hedgehog therapeutic is an N-terminal fragment of the mature protein having a molecular weight of approximately 19 kDa.

Preferred human hedgehog proteins include N-terminal fragments corresponding approximately to residues 24-197 of SEQ ID No. 15, 28-202 of SEQ ID No. 16, and 23-198 of SEQ ID No. 17. By “corresponding approximately” it is meant that the sequence of interest is at most 20 amino acid residues different in length to the reference sequence, though more preferably at most 5, 10 or 15 amino acid different in length.

Still other preferred hedgehog polypeptides includes an amino acid sequence represented by the formula A-B wherein: (i) A represents all or the portion of the amino acid sequence designated by residues 1-168 of SEQ ID No:21; and B represents at least one amino acid residue of the amino acid sequence designated by residues 169-221 of SEQ ID No:21; (ii) A represents all or the portion of the amino acid sequence designated by residues 24-193 of SEQ ID No:15; and B represents at least one amino acid residue of the amino acid sequence designated by residues 194-250 of SEQ ID No: 15; (iii) A represents all or the portion of the amino acid sequence designated by residues 25-193 of SEQ ID No:13; and B represents at least one amino acid residue of the amino acid sequence designated by residues 194-250 of SEQ ID No:13; (iv) A represents all or the portion of the amino acid sequence designated by residues 23-193 of SEQ ID No: 11; and B represents at least one amino acid residue of the amino acid sequence designated by residues 194-250 of SEQ ID No: 11; (v) A represents all or the portion of the amino acid sequence designated by residues 28-197 of SEQ ID No:12; and B represents at least one amino acid residue of the amino acid sequence designated by residues 198-250 of SEQ ID No; 12; (vi) A represents all or the portion of the amino acid sequence designated by residues 29-197 of SEQ ID No: 16; and B represents at least one amino acid residue of the amino acid sequence designated by residues 198-250 of SEQ ID No: 16; or (vii) A represents all or the portion of the amino acid sequence designated by residues 23-193 of SEQ ID No. 17, and B represents at least one amino acid residue of the amino acid sequence designated by residues 194-250 of SEQ ID No. 17. In certain preferred embodiments, A and B together represent a contiguous polypeptide sequence designated sequence, A represents at least 25, 50, 75, 100, 125 or 150 amino acids of the designated sequence, and B represents at least 5, 10, or 20 amino acid residues of the amino acid sequence designated by corresponding entry in the sequence listing, and A and B together preferably represent a contiguous sequence corresponding to the sequence listing entry. Similar fragments from other hedgehog also contemplated, e.g., fragments which correspond to the preferred fragments from the sequence listing entries which are enumerated above.

Generally, the structure of a preferred conjugated hedgehog protein of this invention has the general formula: A—[Sp]—B—[Sp]—X, where A is a polyalkylene glycol polymer moiety; [Sp] is an optional spacer peptide sequence; B is a hedgehog protein (which optionally may have another spacer peptide sequence); and X is an optional hydrophobic moiety linked (optionally by way of the spacer peptide) to the hedgehog protein B or another residue such as a surface site of the protein.

IV. Production of Recombinant Polypeptides

The isolated hedgehog polypeptides described herein may be produced by any suitable method known in the art. Such methods range from direct protein synthetic methods to constructing a DNA sequence encoding isolated polypeptide sequences and expressing those sequences in a suitable transformed host.

In one embodiment of a recombinant method, a DNA sequence is constructed by isolating or synthesizing a DNA sequence encoding a wild type protein of interest. Optionally, the sequence may be mutagenized by site-specific mutagenesis to provide functional analogs thereof. Another method of constructing a DNA sequence encoding a polypeptide of interest would be by chemical synthesis using an oligonucleotide synthesizer. Such oligonucleotides may be preferably designed based on the amino acid sequence of the desired polypeptide, and preferably selecting those codons that are favored in the host cell in which the recombinant polypeptide of interest will be produced. Standard methods may be applied to synthesize an isolated polynucleotide sequence encoding an isolated polypeptide of interest. Once assembled (by synthesis, site-directed mutagenesis, or by another method), the mutant DNA sequences encoding a particular isolated polypeptide of interest will be inserted into an expression vector and operatively linked to an expression control sequence appropriate for expression of the protein in a desired host. Proper assembly may be confirmed by nucleotide sequencing, restriction mapping, and expression of a biologically active polypeptide in a suitable host.

As is well known in the art, in order to obtain high expression levels of a transfected gene in a host, the gene must be operatively linked to transcriptional and translational expression control sequences that are functional in the chosen expression host. As described in the literature, genes for other hedgehog proteins, e.g., from other animals, can be obtained from mRNA or genomic DNA samples using techniques well known in the art. For example, a cDNA encoding a hedgehog protein can be obtained by isolating total mRNA from a cell, e.g. a mammalian cell, e.g. a human cell, including embryonic cells. Double stranded cDNAs can then be prepared from the total mRNA, and subsequently inserted into a suitable plasmid or bacteriophage vector using any one of a number of known techniques. The gene encoding a hedgehog protein can also be cloned using established polymerase chain reaction techniques.

The choice of expression control sequence and expression vector will depend upon the choice of host. A wide variety of expression host/vector combinations may be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Esherichia coli, including pCR1, pBR322, pMB9 and their derivatives, and phage such as M13 and filamentous single-stranded DNA phages. Preferred E. coli vectors include pL vectors containing the lambda phage pL promoter (U.S. Pat. No. 4,874,702), pET vectors containing the T7 polymerase promoter (Studier et al., Methods in Enzymology 185: 60-89, 1990) and the pSP72 vector (Kaelin et al., supra). Useful expression vectors for yeast cells, for example, include the 2μ and centromere plasmids.

In addition, any of a wide variety of expression control sequences may be used in these vectors. Such useful expression control sequences include the expression control sequences associated with structural genes of the foregoing expression vectors. Examples of useful expression control sequences include, for example, the early and late promoters of SV40 or adenovirus, the lac system, the tip system, the TAC or TRC system, the major operator and promoter regions of phage lambda, for example pL, the control regions of fd coat protein, the promoter for 3-phosphoglycerate kinase or other glycolytic enzymes, the promoters of acid phosphatase, e.g., Pho5, the promoters of the yeast alpha-mating system and other sequences known to control the expression of genes of prokaryotic or eukaryotic cells and their viruses, and various combinations thereof.

Any suitable host may be used to produce in quantity the isolated hedgehog polypeptides described herein, including bacteria, fungi (including yeasts), plants, insects, mammals, or other appropriate animal cells or cell lines, as well as transgenic animals or plants. More particularly, these hosts may include well known eukaryotic and prokaryotic hosts, such as strains of E. coli, Pseudomonas, Bacillus, Streptomyces, fungi, yeast (e.g., Hansenula, Pichia), insect cells such as Spodoptera frugiperda (SF9), and High Five™ (see Example 1), animal cells such as Chinese hamster ovary (CHO), mouse cells such as NS/O cells, African green monkey cells COS 1, COS 7, BSC 1, BSC 40, EBNA 293, and BMT 10, and human cells, as well as plant cells.

It should be understood that not all vectors and expression control sequences will function equally well to express a given isolated polypeptide. Neither will all hosts function equally well with the same expression system. However, one of skill in the art may make a selection among these vectors, expression control systems and hosts without undue experimentation. For example, to produce isolated polypeptide of interest in large-scale animal culture, the copy number of the expression vector must be controlled. Amplifiable vectors are well known in the art. See, for example, Kaufman and Sharp, (1982) Mol. Cell. Biol., 2, 1304-1319 and U.S. Pat. Nos. 4,470,461 and 5,122,464.

Such operative linking of a DNA sequence to an expression control sequence includes the provision of a translation start signal in the correct reading frame upstream of the DNA sequence. If the particular DNA sequence being expressed does not begin with a methionine, the start signal may result in an additional amino acid (methionine) being located at the N-terminus of the product. If a hydrophobic moiety is to be linked to the N-terminal methionyl-containing protein, the protein may be employed directly in the compositions of the invention. Neverthless, since the preferred N-terminal end of the protein is to consist of a cysteine (or functional equivalent) the methionine must be removed before use. Methods are available in the art to remove such N-terminal methionines from polypeptides expressed with them. For example, certain hosts and fermentation conditions permit removal of substantially all of the N-terminal methionine in vivo. Other hosts require in vitro removal of the N-terminal methionine. Such in vitro and in vivo methods are well known in the art.

The proteins produced by a transformed host can be purified according to any suitable method. Such standard methods include chromatography (e.g., ion exchange, affinity, and sizing column chromatography), centrifugation, differential solubility, or by any other standard technique for protein purification. For immunoaffinity chromatography (See Example 1), a protein such as Sonic hedgehog (or peptides derived from Sonic hedgehog) may be isolated by binding it to an affinity column comprising of antibodies that were raised against Sonic hedgehog, or a related protein and were affixed to a stationary support. Alternatively, affinity tags such as hexahistidine, maltose binding domain, influenza coat sequence, and glutathione-S-transferase can be attached to the protein to allow easy purification by passage over an appropriate affinity column. Isolated proteins can also be initially characterized using mass spectrometry, electrophoresis (SDS-PAGE) and other conventional methods (e.g., SEC). Isolated proteins can also be characterized physically using such techniques as proteolysis, nuclear magnetic resonance, and X-ray crystallography.

A. Production of Fragments and Analogs

Fragments of an isolated protein (e.g., fragments of SEQ ID NOS: 23-26) can also be produced efficiently by recombinant methods, by proteolytic digestion, or by chemical synthesis using methods known to those of skill in the art. In recombinant methods, internal or terminal fragments of a polypeptide can be generated by removing one or more nucleotides from one end (for a terminal fragment) or both ends (for an internal fragment) of a DNA sequence which encodes for the isolated hedgehog polypeptide. Expression of the mutagenized DNA produces polypeptide fragments. Digestion with “end nibbling” endonucleases can also generate DNAs which encode an array of fragments. DNAs which encode fragments of a protein can also be generated by random shearing, restriction digestion, or a combination or both. Protein fragments can be generated directly from intact proteins. Peptides can be cleaved specifically by proteolytic enzymes, including, but not limited to plasmin, thrombin, trypsin, chymotrypsin, or pepsin. Each of these enzymes is specific for the type of peptide bond it attacks. Trypsin catalyzes the hydrolysis of peptide bonds in which the carbonyl group is from a basic amino acid, usually arginine or lysine. Pepsin and chymotrypsin preferentially catalyse the hydrolysis of peptide bonds from aromatic amino acids, such as tryptophan, tyrosine, and phenylalanine or certain hydrophobic amino acids. Alternative sets of cleaved protein fragments are generated by preventing cleavage at a site which is suceptible to a proteolytic enzyme. For instance, reaction of the ε-amino acid group of lysine with ethyltrifluorothioacetate in mildly basic solution yields blocked amino acid residues whose adjacent peptide bond is no longer susceptible to hydrolysis by trypsin. Proteins can be modified to create peptide linkages that are susceptible to proteolytic enzymes. For instance, alkylation of cysteine residues with β-haloethylamines yields peptide linkages that are hydrolyzed by trypsin (Lindley, (1956) Nature 178, 647). In addition, chemical reagents that cleave peptide chains at specific residues can be used. For example, cyanogen bromide cleaves peptides at methionine residues (Gross and Witkip, (1961) J. Am. Chem. Soc. 83, 1510). Thus, by treating proteins with various combinations of modifiers, proteolytic enzymes and/or chemical reagents, the proteins may be divided into fragments of a desired length with no overlap of the fragments, or divided into overlapping fragments of a desired length.

Fragments may also be synthesized chemically using techniques known in the art such as the Merrifield solid phase F moc or t-Boc chemistry. Merrifield, Recent Progress in Hormone Research 23: 451 (1967)

Examples of prior art methods which allow production and testing of fragments and analogs are discussed below. These, or analogous methods may be used to make and screen fragments and analogs of an isolated polypeptide (e.g., hedgehog) which can be shown to have biological activity. An exemplary method to test whether fragments and analogs of hedgehog have biological activity is found in Example 3.

B. Production of Altered DNA and Peptide Sequences: Random Methods

Amino acid sequence variants of a hedgehog protein may be prepared by random mutagenesis of DNA which encodes the protein or a particular portion thereof. Useful methods include PCR mutagenesis and saturation mutagenesis. A library of random amino acid sequence variants can also be generated by the synthesis of a set of degenerate oligonucleotide sequences. Methods of generating amino acid sequence variants of a given protein using altered DNA and peptides are well-known in the art. The following examples of such methods are not intended to limit the scope of the present invention, but merely serve to illustrate representative techniques. Persons having ordinary skill in the art will recognize that other methods are also useful in this regard.

PCR Mutagenesis: See, for example Leung et al., (1989) Technique 1, 11-15.

Saturation Mutagenesis: One method is described generally in Mayers et al., (1989) Science 229, 242.

Degenerate Oligonucleotide Mutagnesis: See for example Harang, S. A., (1983) Tetrahedron 39, 3; Itakura et al., (1984) Ann. Rev. Biochem. 53, 323 and Itakura et al., Recombinant DNA, Proc. 3rd Cleveland Symposium on Macromolecules, pp. 273-289 (A. G. Walton, ed.), Elsevier, Amsterdam, 1981.

C. Production of Altered DNA and Peptide Sequences: Directed Methods

Non-random, or directed, mutagenesis provides specific sequences or mutations in specific portions of a polynucleotide sequence that encodes an isolated polypeptide, to provide variants which include deletions, insertions, or substitutions of residues of the known amino acid sequence of the isolated polypeptide. The mutation sites may be modified individually or in series, for instance by: (1) substituting first with conserved amino acids and then with more radical choices depending on the results achieved; (2) deleting the target residue; or (3) inserting residues of the same or a different class adjacent to the located site, or combinations of options 1-3.

Clearly, such site-directed methods are one way in which an N-terminal cysteine (or a functional equivalent) can be introduced into a given polypeptide sequence to provide the attachment site for a hydrophobic moiety or specific sites for cysteine residues can be inserted into the sequence to serve as targets for polymer conjugation.

Alanine scanning Mutagenesis: See Cunningham and Wells, (1989) Science 244, 1081-1085).

Oligonucleotide-Mediated Mutagenesis: See, for example, Adelman et al., (1983) DNA 2, 183. Cassette Mutagenesis: See Wells et al., (1985) Gene 34, 315.

Combinatorial Mutagenesis: See, for example, Ladner et al., WO 88/06630. It is plain from the combinatorial mutagenesis art that large scale mutagenesis of hedgehog proteins, without any preconceived ideas of which residues were critical to the biological function, and generate wide arrays of variants having equivalent biological activity. Indeed, it is the ability of combinatorial techniques to screen billions of different variants by high throughout analysis that removes any requirement of a priori understanding or knowledge of critical residues.

To illustrate, the amino acid sequences for a population of hedgehog homologs or other related proteins are aligned, preferably to promote the highest homology possible. Such a population of variants can include, for example, hedgehog homologs from one or more species. Amino acids which appear at each position of the aligned sequences are selected to create a degenerate set of combinatorial sequences. In a preferred embodiment, the variegated library of hedgehog variants is generated by combinatorial mutagenesis at the nucleic acid level, and is encoded by a variegated gene library. For instance, a mixture of synthetic oligonucleotides can be enzymatically ligated into gene sequences such that the degenerate set of potential hedgehog sequences are expressible as individual polypeptides, or alternatively, as a set of larger fusion proteins (e.g. for phage display) containing the set of hedgehog sequences therein.

As illustrated in PCT publication WO 95/18856, to analyze the sequences of a population of variants, the amino acid sequences of interest can be aligned relative to sequence homology. The presence or absence of amino acids from an aligned sequence of a particular variant is relative to a chosen consensus length of a reference sequence, which can be real or artificial.

In an illustrative embodiment, alignment of exons 1, 2 and a portion of exon 3 encoded sequences (e.g. the N-terminal approximately 221 residues of the mature protein) of each of the Shh clones produces a degenerate set of Shh polypeptides represented by the general formula:

| | |
|---|---|
| C-G-P-G-R-G-X(1)-G-X(2)-R-R-H-P-K-K-L-T-P-L-A-Y-K-Q-F-I-P- | (SEQ ID No. 21) |
| N-V-A-E-K-T-L-G-A-S-G-R-Y-E-G-K-I-X(3)-R-M-S-E-R-F-K-E-L- | |
| T-P-N-Y-N-P-D-I-I-F-K-D-E-E-N-T-G-A-D-R-L-M-T-Q-R-C-K-D-K- | |
| L-N-X(4)-L-A-I-S-V-M-N-X(5)-W-P-G-V-X(6)-L-R-V-T-E-G-W-D- | |
| E-D-G-H-H-X(7)-E-E-S-L-H-Y-E-G-R-A-V-D-I-T-T-S-D-R-D-X(8)- | |
| S-K-Y-G-X(9)-L-X(10)-R-L-A-V-E-A-G-F-D-W-V-Y-Y-E-S-K-A-H- | |
| I-H-C-S-V-K-A-E-N-S-V-A-A-K-S-G-G-C-F-P-G-S-A-X(11)-V- | |
| X(12)-L-X(13)-X(14)-G-G-X(15)-K-X-(16)-V-K-D-D-X(17)-P-G- | |
| D-X(18)-V-L-A-A-D-X(19)-X(20)-G-X(21)-L-X(22)-X(23)-S-D-F- | |
| X(24)-X(25)-F-X(26)-D-R, | |

wherein each of the degenerate positions “X” can be an amino acid which occurs in that position in one of the human, mouse, chicken or zebrafish Shh clones, or, to expand the library, each X can also be selected from amongst amino acid residue which would be conservative substitutions for the amino acids which appear naturally in each of those positions. For instance, Xaa(1) represents Gly, Ala, Val, Leu, Ile, Phe, Tyr or Trp Xaa(2) represents Arg, His or Lys; Xaa(3) represents Gly, Ala, Val, Leu, Ile, Ser or Thr; Xaa(4) represents Gly, Ala, Val, Leu, Ile, Ser or Thr; Xaa(5) represents Lys, Arg, His, Asn or Gln; Xaa(6) represents Lys, Arg or His; Xaa(7) represents Ser, Thr, Tyr, Trp or Phe; Xaa(8) represents Lys, Arg or His; Xaa(9) represents Met, Cys, Ser or Thr; Xaa(10) represents Gly, Ala, Val, Leu, Ile, Ser or Thr; Xaa(11) represents Leu, Val, Met, Thr or Ser; Xaa(12) represents His, Phe, Tyr, Ser, Thr, Met or Cys; Xaa(13) represents Gln, Asn, Glu, or Asp; Xaa(14) represents His, Phe, Tyr, Thr, Gln, Asn, Glu or Asp; Xaa(15) represents Gln, Asn, Glu, Asp, Thr, Ser, Met or Cys; Xaa(16) represents Ala, Gly, Cys, Leu, Val or Met; Xaa(17) represents Arg, Lys, Met, Ile, Asn, Asp, Glu, Gln, Ser, Thr or Cys; Xaa(18) represents Arg, Lys, Met or Ile; Xaa(19) represents Ala, Gly, Cys, Asp, Glu, Gln, Asn, Ser, Thr or Met; Xaa(20) represents Ala, Gly, Cys, Asp, Asn, Glu or Gln; Xaa(21) represents Arg, Lys, Met, Ile, Asn, Asp, Glu or Gln; Xaa(22) represent Leu, Val, Met or Ile; Xaa(23) represents Phe, Tyr, Thr, His or Trp; Xaa(24) represents Ile, Val, Leu or Met; Xaa(25) represents Met, Cys, Ile, Leu, Val, Thr or Ser; Xaa(26) represents Leu, Val, Met, Thr or Ser. In an even more expansive library, each X can be selected from any amino acid.

In similar fashion, alignment of each of the human, mouse, chicken and zebrafish hedgehog clones, can provide a degenerate polypeptide sequence represented by the general formula:

| | |
|---|---|
| C-G-P-G-R-G--X(1)-X(2)-X(3)-R-R-X(4)-X(5)-X(6)-P-K-X(7)-L- | (SEQ ID No: 22) |
| X(8)-P-L-X(9)-Y-K-Q-F-X(10)-P-X(11)-X(12)-X(13)-E-X(14)-T- | |
| L-G-A-S-G-X(15)-X(16)-E-G-X(17)-X(18)-X(19)-R-X(20)-S-E-R- | |
| F-X(21)-X(22)-L-T-P-N-Y-N-P-D-I-I-F-K-D-E-E-N-X(23)-G-A-D- | |
| R-L-M-T-X(24)-R-C-K-X(25)-X(26)-X(27)-N-X(28)-L-A-I-S-V-M- | |
| N-X(29)-W-P-G-V-X(30)-L-R-V-T-E-G-X(31)-D-E-D-G-H-H-X(32)- | |
| X(33)-X(34)-S-L-H-Y-E-G-R-A-X(35)-D-I-T-T-S-D-R-D-X(36)- | |
| X(37)-K-Y-G-X(38)-L-X(39)-R-L-A-V-E-A-G-F-D-W-V-Y-Y-E-S- | |
| X(40)-X(41)-H-X(42)-H-X(43)-S-V-K, | |

wherein, as above, each of the degenerate positions “X” can be an amino acid which occurs in a corresponding position in one of the wild-type clones, and may also include amino acid residue which would be conservative substitutions, or each X can be any amino acid residue. In an exemplary embodiment, Xaa(1) represents Gly, Ala, Val, Leu, Ile, Pro, Phe or Tyr; Xaa(2) represents Gly, Ala, Val, Leu or Ile; Xaa(3) represents Gly, Ala, Val, Leu, Ile, Lys, His or Arg; Xaa(4) represents Lys, Arg or His; Xaa(5) represents Phe, Trp, Tyr or an amino acid gap; Xaa(6) represents Gly, Ala, Val, Leu, Ile or an amino acid gap; Xaa(7) represents Asn, Gln, His, Arg or Lys; Xaa(8) represents Gly, Ala, Val, Leu, Ile, Ser or Thr; Xaa(9) represents Gly, Ala, Val, Leu, Ile, Ser or Thr; Xaa(10) represents Gly, Ala, Val, Leu, Ile, Ser or Thr; Xaa(11) represents Ser, Thr, Gln or Asn; Xaa(12) represents Met, Cys, Gly, Ala, Val, Leu, Ile, Ser or Thr; Xaa(13) represents Gly, Ala, Val, Leu, Ile or Pro; Xaa(14) represents Arg, His or Lys; Xaa(15) represents Gly, Ala, Val, Leu, Ile, Pro, Arg, His or Lys; Xaa(16) represents Gly, Ala, Val, Leu, Ile, Phe or Tyr; Xaa(17) represents Arg, His or Lys; Xaa(18) represents Gly, Ala, Val, Leu, Ile, Ser or Thr; Xaa(19) represents Thr or Ser; Xaa(20) represents Gly, Ala, Val, Leu, Ile, Asn or Gln; Xaa(21) represents Arg, His or Lys; Xaa(22) represents Asp or Glu; Xaa(23) represents Ser or Thr; Xaa(24) represents Glu, Asp, Gln or Asn; Xaa(25) represents Glu or Asp; Xaa(26) represents Arg, His or Lys; Xaa(27) represents Gly, Ala, Val, Leu or Ile; Xaa(28) represents Gly, Ala, Val, Leu, Ile, Thr or Ser; Xaa(29) represents Met, Cys, Gln, Asn, Arg, Lys or His; Xaa(30) represents Arg, His or Lys; Xaa(31) represents Trp, Phe, Tyr, Arg, His or Lys; Xaa(32) represents Gly, Ala, Val, Leu, Ile, Ser, Thr, Tyr or Phe; Xaa(33) represents Gln, Asn, Asp or Glu; Xaa(34) represents Asp or Glu; Xaa(35) represents Gly, Ala, Val, Leu, or Ile; Xaa(36) represents Arg, His or Lys; Xaa(37) represents Asn, Gln, Thr or Ser; Xaa(38) represents Gly, Ala, Val, Leu, Ile, Ser, Thr, Met or Cys; Xaa(39) represents Gly, Ala, Val, Leu, Ile, Thr or Ser; Xaa(40) represents Arg, His or Lys; Xaa(41) represents Asn, Gln, Gly, Ala, Val, Leu or Ile; Xaa(42) represents Gly, Ala, Val, Leu or Ile; Xaa(43) represents Gly, Ala, Val, Leu, Ile, Ser, Thr or Cys; Xaa(44) represents Gly, Ala, Val, Leu, Ile, Thr or Ser; and Xaa(45) represents Asp or Glu.

There are many ways by which the library of potential hedgehog homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential hedgehog sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis of hedgehog homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate hedgehog sequences created by combinatorial mutagenesis techniques.

D. Other Variants of Isolated Polypeptides

Included in the invention are isolated molecules that are: allelic variants, natural mutants, induced mutants, and proteins encoded by DNA that hybridizes under high or low stringency conditions to a nucleic acid which encodes a polypeptide such as the N-terminal fragment of Sonic hedgehog (SEQ ID NO: 1) and polypeptides bound specifically by antisera to hedgehog peptides, especially by antisera to an active site or binding site of hedgehog. All variants described herein are expected to: (i) retain the biological function of the original protein and (ii) retain the ability to link to at least one polyalkylene glycol moiety (e.g., a PEG).

The methods of the invention also feature uses of fragments, preferably biologically active fragments, or analogs of an isolated peptide such as hedgehog. Specifically, a biologically active fragment or analog is one having any in vivo or in vitro activity which is characteristic of the peptide shown, for example, in SEQ ID NOS: 23-26 or of other naturally occurring isolated hedgehog. Most preferably, the hydrophobically-modified fragment or analog has at least 10%, preferably 40% or greater, or most preferably at least 90% of the activity of Sonic hedgehog (See Example 3) in any in vivo or in vitro assay.

Analogs can differ from naturally occurring isolated protein in amino acid sequence or in ways that do not involve sequence, or both. The most preferred polypeptides of the invention have preferred non-sequence modifications that include in vivo or in vitro chemical derivatization (e.g., of their N-terminal end), as well as possible changes in acetylation, methylation, phosphorylation, amidation, carboxylation, or glycosylation.

Other analogs include a protein such as Sonic hedgehog or its biologically active fragments whose sequences differ from the wild type consensus sequence (e.g., SEQ ID NOS: 21 or 26) by one or more conservative amino acid substitutions or by one or more non conservative amino acid substitutions, or by deletions or insertions which do not abolish the isolated protein's biological activity. Conservative substitutions typically include the substitution of one amino acid for another with similar characteristics such as substitutions within the following groups: valine, alanine and glycine; leucine and isoleucine; aspartic acid and glutamic acid; asparagine and glutamine; serine and threonine; lysine and arginine; and phenylalanine and tyrosine. The non-polar hydrophobic amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Other conservative substitutions can be readily known by workers of ordinary skill. For example, for the amino acid alanine, a conservative substitution can be taken from any one of D-alanine, glycine, beta-alanine, L-cysteine, and D-cysteine. For lysine, a replacement can be any one of D-lysine, arginine, D-arginine, homo-arginine, methionine, D-methionine, ornithine, or D-ornithine.

Other analogs used within the methods of the invention are those with modifications which increase peptide stability. Such analogs may contain, for example, one or more non-peptide bonds (which replace the peptide bonds) in the peptide sequence. Also included are: analogs that include residues other than naturally occurring L-amino acids, such as D-amino acids or non-naturally occurring or synthetic amino acids such as beta or gamma amino acids and cyclic analogs. Incorporation of D- instead of L-amino acids into the isolated hedgehog polypeptide may increase its resistance to proteases. See, U.S. Pat. No. 5,219,990 supra.

The term “fragment”, as applied to an isolated hedgehog analog, can be as small as a single amino acid provided that it retains biological activity. It may be at least about 20 residues, more typically at least about 40 residues, preferably at least about 60 residues in length. Fragments can be generated by methods known to those skilled in the art. The ability of a candidate fragment to exhibit isolated hedgehog biological activity can be also assessed by methods known to those skilled in the art as described herein.

Production of anti-Hedgehog Antibody Homologs

The technology for producing monoclonal antibody homologs is well known. Briefly, an immortal cell line (typically myeloma cells) is fused to lymphocytes (typically splenocytes) from a mammal immunized with whole cells expressing a given antigen, e.g., hedgehog, and the culture supernatants of the resulting hybridoma cells are screened for antibodies against the antigen. See, generally, Kohler et at., 1975, Nature 265: 295-497, “Continuous Cultures of Fused Cells Secreting Antibody of Predefined Specificity”. Several mouse anti-hedgehog monoclonal antibodies have been described in the prior art.

Fully human monoclonal antibody homologs against hedgehog or patched are another preferred binding agent which may block or coat hedgehog or patched antigens in the method of the invention. In their intact form these may be prepared using several methods: using in vitro-primed human splenocytes (Boemer et al., 1991, J. Immunol. 147:86-95, “Production of Antigen-specific Human Monoclonal Antibodies from In Vitro-Primed Human Splenocytes”); made by repertoire cloning (Persson et al., 1991, Proc. Nat. Acad. Sci. USA 88: 2432-2436, “Generation of diverse high-affinity human monoclonal antibodies by repertoire cloning” and Huang and Stollar, 1991, J. Immunol. Methods 141: 227-236, “Construction of representative immunoglobulin variable region CDNA libraries from human peripheral blood lymphocytes without in vitro stimulation”); prepared from human B cells (U.S. Pat. No. 5,798,230 (Aug. 25, 1998, “Process for the preparation of human monoclonal antibodies and their use”).

In yet another method for producing fully human antibodies, U.S. Pat. No. 5,789,650 (Aug. 4, 1998, “Transgenic non-human animals for producing heterologous antibodies”) describes transgenic non-human animals capable of producing heterologous antibodies and transgenic non-human animals having inactivated endogenous immunoglobulin genes. Endogenous immunoglobulin genes are suppressed by antisense polynucleotides and/or by antiserum directed against endogenous immunoglobulins. Heterologous antibodies are encoded by immunoglobulin genes not normally found in the genome of that species of non-human animal. One or more transgenes containing sequences of unrearranged heterologous human immunoglobulin heavy chains are introduced into a non-human animal thereby forming a transgenic animal capable of functionally rearranging transgenic immunoglobulin sequences and producing a repertoire of antibodies of various isotypes encoded by human immunoglobulin genes. Such heterologous human antibodies are produced in B-cells, which are thereafter immortalized, e.g., by fusing with an immortalizing cell line such as a myeloma or by manipulating such B-cells by other techniques to perpetuate a cell line capable of producing a monoclonal heterologous, fully human antibody homolog.

Yet another preferred binding agent which may block or coat hedgehog or patched antigens in the method of the invention is a humanized recombinant antibody homolog having the capability of binding to a hedgehog or patched protein. See EP 0239400 (Winter et al.) whereby antibodies are altered by substitution of their complementarily determining regions (CDRs) for one species with those from another. This process may be used, for example, to substitute the CDRs from human heavy and light chain Ig variable region domains with alternative CDRs from murine variable region domains. These altered Ig variable regions may subsequently be combined with human Ig constant regions to created antibodies which are totally human in composition except for the substituted murine CDRs. The process for humanizing monoclonal antibodies via CDR “grafting” has been termed “reshaping”. (Riechmann et al., 1988 Nature 332: 323-327, “Reshaping human antibodies for therapy”; Verhoeyen et al., 1988, Science 239: 1534-1536, “Reshaping of human antibodies using CDR-grafting in Monoclonal Antibodies”. The reason that CDR-grafting is successful is that framework regions between mouse and human antibodies may have very similar 3-D structures with similar points of attachment for CDRS, such that CDRs can be interchanged. Such humanized antibody homologs may be prepared, as exemplified in Jones et al., 1986 Nature 321: 522-525, “Replacing the complementarity-determining regions in a human antibody with those from a mouse”; Riechmann, 1988, Nature 332:323-327, “Reshaping human antibodies for therapy”; Queen et al., 1989, Proc. Nat. Acad. Sci. USA 86:10029, “A humanized antibody that binds to the interleukin 2 receptor” and Orlandi et al., 1989, Proc. Natl. Acad. Sci. USA 86:3833 “Cloning Immunoglobulin variable domains for expression by the polymerase chain reaction”. See also Protein Design Labs U.S. Pat. No. 5,585,089 and Tempest (1991, Biotechnology 9: 266-271, “Reshaping a human monoclonal antibody to inhibit human respiratory syncytial virus infection in vivo”)

Regardless of the approach taken, the examples of the initial humanized antibody homologs prepared to date have shown that it is not a straightforward process. Results thus far indicate that changes necessary to preserve specificity and/or affinity are for the most part unique to a given antibody and cannot be predicted based on the humanization of a different antibody. Preferred antagonists useful in the present invention include chimeric recombinant and humanized recombinant antibody homologs (i.e., intact immunoglobulins and portions thereof) with hedgehog or patched specificity.

Hedgehog Proteins as Antagonists

In one preferred embodiment, the hedgehog proteins that are conjugated to a polyalkylene glycol moiety are antagonists of a biological activity of the naturally occurring or recombinant hedgehog protein (e.g., an isolated hedgehog such as a member of the vertebrate family obtainable from Sonic, Indian or Desert hedgehog protein), as defined above.

The antagonists of the present invention are obtainable from isolated hedgehog proteins. Sonic, Indian or Desert may be converted into antagonists, as disclosed in U.S. patent application Ser. No. 60/106,703 (Nov. 2, 1998). Other antagonists are anti-hedgehog or anti-patched-1 antibodies.

A preferred antagonist has at least the following properties: (i) the isolated protein binds the receptor patched-1 with an affinity that may be less than, but is preferably at least the same as, the binding of mature hedgehog protein to patched-1; and (ii) the isolated protein blocks alkaline phosphatase (AP) induction by mature hedgehog protein when tested in an in vitro CH310T1/2 cell-based AP induction assay. Antagonists of the invention may also have the additional properties of being (iii) unable to induce ptc-1 and gli-1 expression.

Persons having ordinary skill in the art can easily test any putative hedgehog antagonist for these properties. In particular, the mouse embryonic fibroblast line C3H10T1/2 is a mesenchymal stem cell line that is hedgehog responsive (as described in more detail in the Examples). Hedgehog treatment of the cells causes an upregulation of gli-1 and patched-1 (known indicators of hedgehog dependent signaling) and also causes induction of alkaline phosphatase activity, an indicator that the cells have differentiated down the chondrocyte/ bone osteoblast lineage. Several hedgehog variants are unable to elicit a hedgehog-dependent response on C3H10T1/2 cells, but they competed with mature hedgehog for function and therefore served as functional antagonists. These functional antagonists are particularly preferred as the hedgehog to which a polyalkylene glycol moiety is conjugated. Their synthesis and use are briefly described below.

A. N-Modified Hedgehog Polypeptides as Antagonists

Certain hedgehog variants that contain N-terminal modifications can block hedgehog function because they lack the ability to elicit a hedgehog-dependent response but retain the ability to bind to hedgehog receptor, patched-1. The critical primary amino acid sequence that defines whether a hedgehog polypeptide (i.e., a Sonic, Indian or Desert hedgehog) is a functional hedgehog antagonist is the N-terminal cysteine residue which corresponds to Cys-1 of the mature hedgehog. So long as the hedgehog polypeptide either lacks this N-terminal cysteine completely or contains this N-terminal cysteine in a modified form (e.g. chemically modified or included as part of an N-terminal extension moiety), the resulting polypeptide can act as a functional hedgehog antagonist. In this regard, the fact that an N-terminal cysteine “corresponds to Cys-1” means: (a) the N-terminal cysteine is the Cys-1 of mature Sonic, Indian or Desert hedgehog; or (b) the N-terminal cysteine occupies the same position as Cys-1 of mature Sonic, Indian or Desert hedgehog. Provided that, for example, a Sonic hedgehog has an N-terminal cysteine corresponding to Cys-1 that is altered or otherwise modified as described herein, it can antagonize the action of any other member of the hedgehog family. Therefore, persons having ordinary skill in the art will understand that it is possible to an Indian hedgehog protein that antagonizes the activity of Sonic, Desert or Indian hedgehogs.

Examples of these antagonists with N-terminal modifications are included below and one skilled in the art can alter the disclosed structure of the antagonist, e.g., by producing fragments or analogs, and test the newly produced structures for antagonist activity. These examples in no way limit the structure of any related hedgehog antagonists, but are merely provided for further description. These, or analogous methods, can be used to make and screen fragments and analogs of a antagonist polypeptides. There are several variants that are able to function as antagonists.

1. N-terminal Extensions

Antagonist polypeptides of the invention may include a hedgehog polypeptide sequence in which the N-terminal cysteine is linked to an N-terminal extension moiety. The isolated antagonist polypeptide can therefore be, as but one example, a recombinant fusion protein having: (a) a first N-terminal polypeptide portion that can be 5′ to the hedgehog polypeptide itself, and that contains at least one element (e.g., an amino acid residue) that may be unrelated to hedgehog, linked to (b) an N-terminal cysteine corresponding to Cys-1 of Sonic hedgehog that is part of a hedgehog antagonist of the invention, or a portion of hedgehog antagonist. This N-terminal extension moiety (e.g., the first N-terminal polypeptide portion) can be a histidine tag, a maltose binding protein, glutathione-S-transferase, a DNA binding domain, or a polymerase activating domain. The functional antagonist may include an N-terminal extension moiety that contains an element which replaces the Cys-1 of mature hedgehog or an N-terminal cysteine that corresponds to Cys-1 of a mature Sonic hedgehog.

2. N-terminal Deletions

Another variation of a functional antagonist is a hedgehog protein that is missing no greater than about 12 amino acids beginning from that N-terminal cysteine corresponding to Cys-1 of a mature hedgehog. Deletions in more than the about the first 12 contiguous amino acid residues do not generate functional antagonists. Preferably, deletions of about 10 contiguous amino acids will provide suitable functional antagonists. One can, however, remove fewer than 10 contiguous residues and still maintain antagonist function. Moreover, one can delete various combinations of non-contiguous residues provided that there are at least about 3 deleted residues in total.

These structures highlight the importance of the N-terminus of hedgehog proteins for function and indeed, underscore the need to conjugate a hedgehog protein at a site other than the N-terminal cysteine. All of the N-terminal deletion variants were indistinguishable from mature Sonic hedgehog (Shh) in their ability to bind patched-1, but were inactive in the in vitro C3H10T1/2 AP induction assay. All these N-terminal variants are unable to promote hedgehog-dependent signaling.

3. N-terminal Mutations

Yet another functional antagonist has a mutation of the N-terminal cysteine to another amino acid residue. Any non-hydrophobic amino acid residue may acceptable and persons having ordinary skill in the art following the teachings described herein will be able to perform the mutations and test the effects of such mutations. One example is Shh in which the N-terminal cysteine is replaced with a serine residue. This mutated form is indistinguishable from mature Shh in its ability to bind patched-1, but it blocks AP induction by mature Shh when tested for function in the C3H10T1/2 AP induction assay. Replacements with aspartic acid, alanine and histidine have also shown to serve as antagonists.

4. N-terminal Cysteine Modifications

Because the primary amino acid sequence of hedgehog contains the Cys-1 that is important for biological activity, certain other modifications will result in inactive antagonist variants of hedgehog protein. Another antagonist is an isolated functional antagonist of a hedgehog polypeptide, comprising a hedgehog polypeptide containing an N-terminal cysteine that corresponds to Cys-1 of a mature Sonic hedgehog, except that the cysteine is in a modified form. Antagonist polypeptides of hedgehog may have non-sequence modifications that include in vivo or in vitro chemical derivatization of their N-terminal cysteine, as well as possible changes in acetylation, methylation, phosphorylation, amidation, or carboxylation. As an example, the functional antagonist can have an N-terminal cysteine in an oxidized form. Thus, a functional antagonist can have an N-terminal cysteine that is effectively modified by including it as part of an N-terminal extension moiety.

B. Other Embodiments

The functional antagonist polypeptides can include amino acid sequences that are at least 60% homologous to a hedgehog protein. The antagonist must exhibit at least the following functional antagonist properties: (i) the isolated protein binds the receptor patched-1 with an affinity that may be less than, but is preferably at least the same as, the binding of mature hedgehog protein to patched-1; and (ii) the isolated protein blocks alkaline phosphatase (AP) induction by mature hedgehog protein when tested in an in vitro CH310T1/2 cell-based AP induction assay.

Antagonists useful in the present invention also include those which arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

In a preferred embodiment, isolated antagonist is a polypeptide with one or more of the following characteristics:

(i) it has at least 60, more preferably 90 and most preferably 95% sequence identity with amino acids of, for example, SEQ ID NOS: 23-26;

(ii) it either has a modified N-terminal cysteine or lacks an N-terminal cysteine or has an N-terminal cysteine in a position different from the N-terminal cysteine corresponding to Cys-1 of the hedgehog;

(iii) it blocks alkaline phosphatase induction by mature hedgehog in CH310T1/2 cells;

(iv) it binds or interacts with its receptor patched-1 with an affinity that may be less than, but is preferably at least the same as, the binding of mature hedgehog protein to patched-1;

(v) it is unable to induce ptc-1 and gli-1 expression in vitro in CH310T1/2 cells; or

(vi) it is unable to induce AP in CH310T1/2 assays.

Moreover, isolated hedgehog antagonists useful in the present invention can also be a recombinant fusion protein containing additional C-terminal sequences unrelated to hedgehog. Thus, the antagonist polypeptide may also include all or a fragment of an amino acid sequence from, for example, SEQ ID NOS: 23-26, fused, in reading frame, to additional amino acid residues. One version of the polypeptides of the invention is a protein having a first polypeptide portion and a hedgehog antagonist portion, the antagonist portion being fused or otherwise linked either 5′ or 3′ to the first polypeptide portion. Thus, first, additional polypeptide portion has an amino acid sequence unrelated to an antagonist polypeptide. The additional polypeptide portion can be, e.g., any of glutathione-S-transferase, a DNA binding domain, or a polymerase activating domain, a histidine tag, an immunoglobulin or portion thereof, fused or otherwise linked to either the N- or C-terminus of the antagonist portion.

Agonists of Hedgehog Biological Activity

Certain preferred hedgehog polypeptides of the invention are based, in part, on the discovery disclosed in U.S. patent application Ser. No. 60/067,423 (Dec. 3, 1997) that human Sonic hedgehog, expressed as a full-length construct in either insect or in mammalian cells, has a hydrophobic palmitoyl group appended to the alpha-amine of the N-terminal cysteine. This is the first example of an extracellular signaling protein being modified in such a manner, and, in contrast to thiol-linked palmitic acid modifications whose attachment is readily reversible, this novel N-linked palmitoyl moiety is likely to be very stable by analogy with myristic acid modifications.

As a direct consequence of this initial discovery, it is known that increasing the hydrophobic nature of a hedgehog signaling protein can increase the protein's biological activity. Thus, the modified hedgehog acts as its own agonist. In particular, appending a hydrophobic moiety to a signaling protein, such as a hedgehog protein, can enhance the protein's activity, and thus, act as an agonist. The N-terminal cysteine of biologically active proteins not only provides a convenient site for appending a hydrophobic moiety, and thereby modifying the physico-chemical properties of the protein, but modifications to the N-terminal cysteine can also increase the protein's stability. Additionally, addition of a hydrophobic moiety to an internal amino acid residue on the surface of the protein structure enhances the protein's activity. Use of these agonists in conjuction with one or more polyalkylene glycol moieties will allow increased bioavailability of the hedgehog agonists in a therapeutic context.

Accordingly, the methods and compositions of the present invention include the use of the conjugated hedgehog agonists due to their increased biological activity. Moreover, the subject methods can be performed on cells which are provided in culture (in vitro), or on cells in a whole animal (in vivo).

The agonists have at least one of the following properties: (i) the isolated protein binds the receptor patched-1 with an affinity that is at similar to, but is preferably higher than, the binding of mature hedgehog protein to patched-1; or (ii) the isolated protein interacts with its external and internal milieu in such a way as to increase the protein's binding affinity to patched-1 when tested in an in vitro CH310T1/2 cell-based AP induction assay. Agonists of the invention may also have the additional properties of being (iii) able to solely induce ptc-1 and gli-1 expression.

A. General Properties of Isolated Hedgehog Proteins Acting As Agonists

The polypeptide portion of the hedgehog compositions of the subject method can be generated by any of a variety of techniques, including purification of naturally occurring proteins, recombinantly produced proteins and synthetic chemistry. Polypeptide forms of the hedgehog proteins are preferably derived from vertebrate hedgehog proteins, e.g., have sequences corresponding to naturally occurring hedgehog proteins, or fragments thereof, from vertebrate organisms. However, it will be appreciated that the hedgehog polypeptide can correspond to a hedgehog protein (or fragment thereof) which occurs in any metazoan organism.

Family members useful in the methods of the invention include any of the naturally-occurring native hedgehog proteins including allelic, phylogenetic counterparts or other variants thereof, whether naturally-sourced or produced chemically including muteins or mutant proteins, as well as recombinant forms and new, active members of the hedgehog family.

The preferred agonists for use in conjugation with polyalkylene glycol include a derivitized hedgehog polypeptide sequence as well as other N-terminal and/or C-terminal amino acid sequence or it may include all or a fragment of a hedgehog amino acid sequence. The isolated hedgehog polypeptide can also be a recombinant fusion protein having a first hedgehog portion and a second polypeptide portion, e.g., a second polypeptide portion having an amino acid sequence unrelated to hedgehog. The second polypeptide portion can be, e.g., histidine tag, maltose binding protein, glutathione-S-transferase, a DNA binding domain, or a polymerase-activating domain.

Polypeptides of the invention include those that arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

In a preferred embodiment, the agonist to be conjugated is a hedgehog polypeptide with one or more of the following characteristics:

(i) it has at least 30, 40, 42, 50, 60, 70, 80, 90 or 95% sequence identity with a hedgehog sequence;

(ii) it has a cysteine or a functional equivalent as the N-terminal end;

(iii) it may induce alkaline phosphatase activity in C3H10T1/2 cells;

(iv) it has an overall sequence identity of at least 50%, preferably at least 60%, more preferably at least 70, 80, 90, or 95%, with a polypeptide of a hedgehog sequence;

(v) it can be isolated from natural sources such as mammalian cells;

(vi) it can bind or interact with patched; and

(vii) it is hydrophobically-modified (i.e., it has at least one hydrophobic moiety attached to the polypeptide).

Increasing the overall hydrophobic nature of a hedgehog protein increases the biological activity of the protein. The potency of a signaling protein such as hedgehog can be increased by: (a) chemically modifying, such as by adding a hydrophobic moiety to, the sulfhydryl and/or to the alpha-amine of the N-terminal cysteine (see U.S. Ser. No. 60/067,423); (b) replacing the N-terminal cysteine with a hydrophobic amino acid (see U.S. Ser. No. 60/067,423); or (c) replacing the N-terminal cysteine with a different amino acid and then chemically modifying the substituted residue so as to add a hydrophobic moiety at the site of the substitution.

Additionally, modification of a hedgehog protein at an internal residue on the surface of the protein with a hydrophobic moiety by: (a) replacing the internal residue with a hydrophobic amino acid; or (b) replacing the internal residue with a different amino acid and then chemically modifying the substituted residue so as to add a hydrophobic moiety at the site of the substitution will retain or enhance the biological activity of the protein.

Additionally, modification of a protein such as a hedgehog protein at the C-terminus with a hydrophobic moiety by: (a) replacing the C-terminal residue with a hydrophobic amino acid; or (b) replacing the C-terminal residue with a different amino acid and then chemically modifying the substituted residue so as to add a hydrophobic moiety at the site of the substitution, will retain or enhance the biological activity of the protein.

For hydrophobically-modified hedgehog obtained by chemically modifying the soluble, unmodified protein, palmitic acid and other lipids can be added to soluble Shh to create a lipid-modified forms with increased potency in the C3H10T1/2 assay. Another form of protein encompassed by the invention is a protein derivatized with a variety of lipid moieties. The principal classes of lipids that are encompassed within this invention are fatty acids and sterols (e.g., cholesterol). Derivatized proteins of the invention may contain fatty acids which are cyclic, acyclic (i.e., straight chain), saturated or unsaturated, mono-carboxylic acids. Exemplary saturated fatty acids have the generic formula: CH3 (CH2)n COOH. Table 2 below lists examples of some fatty acids that can be derivatized conveniently using conventional chemical methods.

TABLE 2
Exemplary Saturated and Unsaturated Fatty Acids

| Saturated Acids: CH3 (CH2)n COOH: | |
|---|---|
| Value of n | Common Name |
| 2 | butyric acid |
| 4 | caproic acid |
| 6 | caprylic acid |
| 8 | capric acid |
| 10 | lauric acid |
| 12 | myristic acid* |
| 14 | palmitic acid* |
| 16 | stearic acid* |
| 18 | arachidic acid* |
| 20 | behenic acid |
| 22 | lignoceric acid |
| Unsaturated Acids: | |
| CH3CH═CHCOOH | crotonic acid |
| CH3(CH2)3CH═CH(CH2)7COOH | myristoleic acid* |
| CH3(CH2)5CH═CH (CH2)7COOH | palmitoleic acid* |
| CH3(CH2)7CH═CH(CH2)7COOH | oleic acid* |
| CH3(CH2)3(CH2CH═CH)2(CH2)7COOH | linoleic acid |
| CH3(CH2CH═CH)3(CH2)7COOH | linolenic acid |
| CH3(CH2)3(CH2CH═CH)4(CH2)3COOH | arachidonic acid |

The asterisk (*) denotes fatty acids detected in recombinant hedgehog protein secreted from a soluble construct (Pepinsky et al., supra).

Other lipids that can be attached to the protein include branched-chain fatty (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

Testing for Biological Activity

While many bioassays have been used to demonstrate hedgehog activity, the C3H10T1/2 cell line provides a simple system for assessing hedgehog function without the complication of having to work with primary cell cultures or organ explants. The mouse embryonic fibroblast line C3H10T1/2 is a mesenchymal stem cell line that, under defined conditions, can differentiate into adipocytes, chondrocytes, and bone osteoblasts (Taylor, S. M., and Jones, P. A., Cell 17: 771-779 (1979) and Wang, E. A., et al., Growth Factors 9: 57-71 (1993)). Bone morphogenic proteins drive the differentiation of C3H10T1/2 cells into the bone cell lineage and alkaline phosphatase induction has been used as a marker for this process (Wang et al., supra). Shh has a similar effect on C3H10T1/2 cells (Kinto, N. et al., FEBS Letts. 404: 319-323 (1997)) and we routinely use the alkaline phosphatase induction by Shh as a quantitative measure of its in vitro potency. Shh treatment also produces a dose-dependent increase in gli-1 and ptc-1 expression, which can be readily detected by a PCR-based analysis.

### THE POLYMER MOIETY

A. Attachment of a Polymer Moiety to the N-terminal Cysteine of Hedgehog

Hedgehog proteins are conjugated most preferably via a terminal reactive group on the polyalkylene glycol polymer although conjugations can also be branched from non-terminal reactive groups. The polymer with the reactive group(s) is designated herein as “activated polymer”. The reactive group would be expected to selectively react with free amino or other reactive groups on the hedgehog protein. In theory, the activated polymer(s) are reacted so that attachment could occur at any available hedgehog amino group such as alpha amino groups or the epsilon-amino groups of lysines, or —SH groups of cysteines. Free carboxylic groups, suitably activated carbonyl groups, hydroxyl, guanidyl, oxidized carbohydrate moieties and mercapto groups of the hedgehog protein (if available) can also be used as attachment sites.

In particular, the chemical modification of any N-terminal cysteine to protect the thiol, with concomitant conjugation with a polyalkylene glycol moiety (i.e., PEG), can be carried out in numerous ways by someone skilled in the art. See U.S. Pat. No. 4,179,337. The sulfhydryl moiety, with the thiolate ion as the active species, is the most reactive functional group in a protein. There are many reagents that react faster with the thiol than any other groups. See Chemistry of Protein Conjugation and Cross-Linking (S. S. Wong, CRC Press, Boca Raton, Fla., 1991). The thiol of an N-terminal cysteine, such as found in all hedgehog proteins, would be expected to be more reactive than internal cysteines within the sequence. This is because the close proximity to the alpha-amine will lower the pKa of the thiol resulting in a greater degree of proton dissociation to the reactive thiolate ion at neutral or acid pH. In addition, the cysteine at the N-terminus of the structure is more likely to be exposed than the other two cysteines in the hedgehog sequence that are found buried in the protein structure.

Other examples of methods that provide linkage between a polyalkylene glycol and the N-terminal cysteine would be reactions with other alpha-haloacetyl compounds, organomercurials, disulfide reagents, and other N-substituted maleimides. Numerous derivatives of these active species are available commercially (e.g., ethyl iodoacetate (Aldrich, Milwaukee, Wis.), phenyl disulfide (Aldrich), and N-pyrenemaleimide (Molecular Probes, Eugene, Oreg.)) or could be synthesized readily (e.g., N-alkyliodoacetamides, N-alkylmaleimides, and organomercurials). Another aspect to the reactivity of an N-terminal cysteine is that it can take part in reaction chemistries unique to its 1,2-aminothiol configuration. One example is the reaction with thioester groups to form an N-terminal amide group via a rapid S to N shift of the thioester. This reaction chemistry can couple together synthetic peptides and can be used to add single or multiple, natural or unnatural, amino acids or other hydrophobic groups via the appropriately activated peptide. Another example, is the reaction with aldehydes to form the thiazolidine adduct. Numerous hydrophobic derivatives of thiol esters (e.g., C2-C24 saturated and unsaturated fatty acyl Coenzyme A esters (Sigma Chemical Co., St. Louis, Mo.)), aldehydes (e.g., butyraldehyde, n-decyl aldehyde, and n-myristyl aldehyde (Aldrich)), and ketones (e.g., 2-, 3-, and 4-decanone (Aldrich)) are available commercially or could be synthesized readily. In a similar manner, thiomorpholine could be prepared from a variety of alpha-haloketone starting materials.

B. Attachment of a Polymer Moiety to the C-terminus of Hedgehog

Notwithstanding the fact that the chemistry needed to attach a polyalkylene glycol moiety to the N-terminal cysteine or the lysines of hedgehog protein is readily available, we have surprisingly discovered that the activity of the hedgehog is completely eliminated when a polyalkylene glycol is conjugated via the N-terminal cysteine with non-cleavable polyalkylene glycol moieties or is conjugaged through the lysines when the linkage is performed in solution phase (see Example 1A).

As a result, it is not correct to assume that, once a particular “PEG” linkage chemistry is available for a particular amino acid, the attachment of the polymer moiety to that particular amino acid will have the intended effect.

Indeed, although the polymer may be attached anywhere on the hedgehog molecule that is not already modified by, for example, a hydrophobic group, the most preferred site for polymer coupling is at a site other than the N-terminus of the hedgehog and other than the lysine(s). The most preferred sites are site(s) at or near the C-terminus. Several observations suggest that the C-terminus or amino acids near the C-terminus would be preferred targets for modification with a polyalkylene glycol moiety: (i) The wild-type protein is naturally modified with cholesterol at this site, indicating that it is exposed and available for modification. Indeed, we showed that treatment with thrombin results in selective release of the C-terminal 3 amino acids (See U.S. Ser. No. 60/106,703, filed Nov. 2, 1998); (ii) We performed extensive SAR analyses and discovered that the C-terminal 11 amino acids could be deleted without harmful effects on folding or function; (iii) We have made hedgehog/Ig fusion proteins by attaching an Ig moiety to the C-terminus of hedgehog without harmful effects on folding or function (data not presented here).

While there is no simple chemical strategy for targeting a polyalkylene glycol polymer such as PEG to the C-terminus of hedgehog, it is straightforward to genetically engineer a site that can be used to target the polymer moiety, as discussed above with regard to site-directed mutagenesis. For example, incorporation of a Cys at a site that is at or near the C-terminus allows specific modification using a maleimide, vinylsulfone or haloacetate- activated polyalkylene glycol (e.g., PEG). As discussed above in Section A, these derivatives can be used specifically for modification of the engineered C-terminal cysteines due to the high selectively of these reagents for Cys. Other strategies such as incorporation of a histidine tag which can be targeted (Fancy et al., (1996) Chem. & Biol. 3: 551) or an additional glycosylation site, represent other alternatives for modifying the C-terminus of hedgehog.

Within the broad scope of the present invention, a single polymer molecule may be employed for conjugation with the hedgehog protein and modified versions thereof as discussed above, although it is also contemplated that more than one polymer molecule can be attached as well. Conjugated hedgehog compositions of the invention may find utility in both in vivo as well as non-in vivo applications. Additionally, it will be recognized that the conjugating polymer may utilize any other groups, moieties, or other conjugated species, as appropriate to the end use application. By way of example, it may be useful in some applications to covalently bond to the polymer a functional moiety imparting UV-degradation resistance, or antioxidation, or other properties or characteristics to the polymer. As a further example, it may be advantageous in some applications to functionalize the polymer to render it reactive or cross-linkable in character, to enhance various properties or characterisics of the overall conjugated material. Accordingly, the polymer may contain any functionality, repeating groups, linkages, or other constitutent structures which do not preclude the efficacy of the conjugated hedgehog composition for its intended purpose. Other objectives and advantages of the present invention will be more fully apparent from the ensuing disclosure and appended claims.

Illustrative polymers that may usefully be employed to achieve these desirable characteristics are described herein below in exemplary reaction schemes. In covalently bonded peptide applications, the polymer may be functionalized and then coupled to free amino acid(s) of the peptide(s) to form labile bonds.

Generally from about 1.0 to about 10 moles of activated polymer per mole of protein is employed, depending on the particular reaction chemistry and the protein concentration. The final amount is a balance between maximizing the extent of the reaction while minimizing non-specific modifications of the product and, at the same time, defining chemistries that will maintain optimum activity, while at the same time optimizing, if possible, the half-life of the protein. Preferably, at least about 50% of the biological activity of the protein is retained, and most preferably 100% is retained.

The reactions may take place by any suitable method used for reacting biologically active materials with inert polymers. Generally the process involves preparing an activated polymer (that may have at least one terminal hydroxyl group) and thereafter reacting the protein with the activated polymer to produce the soluble protein suitable for formulation. The above modification reaction can be performed by several methods, which may involve one or more steps.

Suitable methods of attaching a polyalkylene glycol moiety to a C-terminal cysteine involve using such moieties that are activated with a thiol reactive group, as generally discussed above. Common thiol reactive groups include maleimides, vinylsulfones or haloacetates. These derivatives can be used specifically for modification of cysteines due to the high selectively of these reagents for —SH. Maleimides react specifically with free sulfhydryls (cysteine residues) in minutes, under slightly acidic to neutral (pH 6.0-7.5) conditions. This pH range is preferred although the reaction will proceed, albeit slowly, at pH 5.0. Halogens (iodoacetyl functions) react with —SH groups at physiological pH's to slightly basic conditions. Both of these reactive groups result in the formation of stable thioether bonds.

In the practice of the present invention, polyalkylene glycol residues of C1-C4 alkyl polyalkylene glycols, preferably polyethylene glycol (PEG), or poly(oxy)alkylene glycol residues of such glycols are advantageously incorporated in the polymer systems of interest. Thus, the polymer to which the protein is attached can be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, polyoxyethylenated glycols, copolymers thereof and block copolymers thereof, provided that the water solubility of the block copolymer is maintained. Examples of polyoxyethylated polyols include, for example, polyoxyethylated glycerol, polyoxyethylated sorbitol, polyoxyethylated glucose, or the like. The glycerol backbone of polyoxyethylated glycerol is the same backbone occurring naturally in, for example, animals and humans in mono-, di-, and triglycerides. Therefore, this branching would not necessarily be seen as a foreign agent in the body.

As an alternative to polyalkylene oxides, dextran, polyvinyl pyrrolidones, polyacrylamides, polyvinyl alcohols, carbohydrate-based polymers and the like may be used. Moreover, heteropolymers (i.e., polymers consisting of more than one species of monomer such as a copolymer) as described in U.S. Pat. No. 5,359,030 may be used (e.g., proteins conjugated to polymers comprising a polyalkylene glycol moiety and one or more fatty acids) Those of ordinary skill in the art will recognize that the foregoing list is merely illustrative and that all polymer materials having the qualities described herein are contemplated.

The polymer need not have any particular molecular weight, but it is preferred that the molecular weight be between about 300 and 100,000, more preferably between 10,000 and 40,000. In particular, sizes of 20,000 or more are best at preventing protein loss due to filtration in the kidneys.

Polyalkylene glycol derivatization has a number of advantageous properties in the formulation of polymer-hedgehog conjugates in the practice of the present invention, as associated with the following properties of polyalkylene glycol derivatives: improvement of aqueous solubility, while at the same time eliciting no antigenic or immunogenic response; high degrees of biocompatibility; absence of in vivo biodegradation of the polyalkylene glycol derivatives; and ease of excretion by living organisms.

Moreover, in another aspect of the invention, one can utilize hedgehog covalently bonded to the polymer component in which the nature of the conjugation involves cleavable covalent chemical bonds. (See Example 1). This allows for control in terms of the time course over which the polymer may be cleaved from the hedgehog. This covalent bond between the hedgehog protein drug and the polymer may be cleaved by chemical or enzymatic reaction. The polymer-hedgehog protein product retains an acceptable amount of activity. Concurrently, portions of polyethylene glycol are present in the conjugating polymer to endow the polymer-hedgehog protein conjugate with high aqueous solubility and prolonged blood circulation capability. As a result of these improved characteristics the invention contemplates parenteral, aerosol, and oral delivery of both the active polymer-hedgehog protein species and, following hydrolytic cleavage, bioavailability of the hedgehog protein per se, in in vivo applications.

It is to be understood that the reaction schemes described herein are provided for the purposes of illustration only and are not to be limiting with respect to the reactions and structures which may be utilized in the modification of the hedgehog protein, e.g., to achieve solubility, stabilization, and cell membrane affinity for parenteral and oral administration. Generally speaking, the concentrations of reagents used are not critical to carrying out the procedures provided hererin except that the molar amount of activated polymer should be at least equal to, and preferably in excess of, the molar amount of the reactive group (e.g., thiol) on the hedgehog amino acid(s). The reaction of the polymer with the hedgehog to obtain the most preferred conjugated products is readily carried out using a wide variety of reaction schemes. The activity and stability of the hedgehog protein conjugates can be varied in several ways, by using a polymer of different molecular size. Solubilities of the conjugates can be varied by changing the proportion and size of the polyethylene glycol fragment incorporated in the polymer composition.

Utilities

The unique property of polyalkylene glycol-derived polymers of value for therapeutic applications of the present invention is their general biocompatibility. The polymers have various water solubility properties and are not toxic. They are believed non-immunogenic and non-antigenic and do not interfere with the biological activities of the hedgehog protein moiety when conjugated under the conditions described herein. They have long circulation in the blood and are easily excreted from living organisms.

The therapeutic polymer conjugates of the present invention may be utilized for the prophylaxis or treatment of any condition or disease state for which the hedgehog protein constituent is efficacious. In addition, the polymer-based conjugates of the present invention may be utilized in diagnosis of constituents, conditions, or disease states in biological systems or specimens, as well as for diagnosis purposes in non-physiological systems.

In therapeutic usage, the present invention contemplates a method of treating an animal subject having or latently susceptible to such condition(s) or disease state(s) and in need of such treatment, comprising administering to such animal an effective amount of a polymer conjugate of the present invention which is therapeutically effective for said condition or disease state. Subjects to be treated by the polymer conjugates of the present invention include mammalian subjects and most preferably human subjects. Depending on the specific condition or disease state to be combated, animal subjects may be administered polymer conjugates of the invention at any suitable therapeutically effective and safe dosage, as may readily be determined within the skill of the art, and without undue experimentation.

Generally, the modified proteins described herein are useful for treating the same medical conditions that can be treated with the unmodified forms of the proteins. As but one example of the application of the proteins of this invention in a therapeutic context, modified hedgehog proteins according to the invention can be administered to patients suffering from a variety of neurological conditions. The ability of hedgehog protein to regulate neuronal differentiation during development of the nervous system and also presumably in the adult state indicates that polymer conjugated hedgehog can reasonably be expected to facilitate control of adult neurons with regard to maintenance, functional performance, and aging of normal cells; repair and regeneration processes in lesioned cells; and prevention of degeneration and premature death which results from loss of differentiation in certain pathological conditions. In light of this, the present modified hedgehog compositions, by treatment with a local infusion can prevent and/or reduce the severity of neurological conditions deriving from: (i) acute, subacute, or chronic injury to the nervous system, including traumatic injury, chemical injury, vessel injury, and deficits (such as the ischemia from stroke), together with infectious and tumor-induced injury; (ii) aging of the nervous system including Alzheimer's disease; (iii) chronic neurodegenerative diseases of the nervous system, including Parkinson's disease, Huntington's chorea, amylotrophic lateral sclerosis and the like; and (iv) chronic immunological diseases of the nervous system, including multiple sclerosis. The modifed hedgehog proteins may also be injected into the cerebrospinal fluid, e.g., in order to address deficiencies of brain cells, or into the lymph system or blood stream as required to target other tissue or organ system-specific disorders.

Hedgehog compositions of the invention may be used to rescue, for example, various neurons from lesion-induced death as well as guiding reprojection of these neurons after such damage. Such damage can be attributed to conditions that include, but are not limited to, CNS trauma infarction, infection, metabolic disease, nutritional deficiency, and toxic agents (such as cisplatin treatment). Certain hedgehog proteins cause neoplastic or hyperplastic transformed cells to become either post-mitotic or apoptotic. Such compositions may, therefore, be of use in the treatment of, for instance, malignant gliomas, medulloblastomas and neuroectodermal tumors.

Modified proteins of the invention can be used to specifically target medical therapies against cancers and tumors which express the receptor for the protein. Such materials can be made more effective as cancer therapeutics by using them as delivery vehicles for antineoplastic drugs, toxins, and cytocidal radionuclides, such as yttrium 90.

A toxin may also be conjugated to the modified hedgehog to selectively target and kill hedgehog-responsive cells, such as a tumor expressing hedgehog receptor(s). Other toxins are equally useful, as known to those of skill in the art. Such toxins include, but are not limited to, Pseudomonas exotoxin, Diphtheria toxin, and saporin. This approach should prove successful because hedgehog receptor(s) are expressed in a very limited number of tissues. Another approach to such medical therapies is to use radioisotope labeled, modified protein. Such radiolabeled compounds will preferentially target radioactivity to sites in cells expressing the protein receptor(s), sparing normal tissues. Depending on the radioisotope employed, the radiation emitted from a radiolabeled protein bound to a tumor cell may also kill nearby malignant tumor cells that do not express the protein receptor. A variety of radionuclides may be used.

It is envisioned that subcutaneous delivery will be the primary route for therapeutic administration of the proteins of this invention. Local, intravenous delivery, or delivery through catheter or other surgical tubing may also be envisioned. Alternative routes include tablets and the like, commercially available nebulizers for liquid formulations, and inhalation of lyophilized or aerosolized formulations. Liquid formulations may be utilized after reconstitution from powder formulations.

For neurodegenerative disorders, several animal models are available that are believed to have some clinical predicative value. For Parkinson's disease, models involve the protection, or the recovery in rodents or primates in which the nigral-striatal dopaminergic pathway is damaged either by the systemic administration of MPTP or the local (intracranial) administration of 6-hydroxydopamine [6-OHDA], two selective dopaminergic toxins. Specific models are: MPTP- treated mouse model (Tomac et al., (1995) Nature 373, 335-339); MPTP-treated primate (marmoset or Rhesus) model (Gash et al., (1996) Nature 380, 252-255) and the unilateral 6-OHDA lesion rat model (Hoffer et al., (1994) Neuroscience Lett. 182, 107-111). For ALS, (Amyotrophic lateral sclerosis) models involve treatment of several mice strains that show spontaneous motor neuron degeneration, including the wobbler (Duchen, L. W. and Strich, S. J., (1968), J. Neurol. Neurosurg. Psychiatry 31, 535-542) and pmn mice (Kennel et al., (1996) Neurobiology of Disease 3, 137-147) and of transgenic mice expressing the human mutated superoxidase dismutase (hSOD) gene that has been linked to familial ALS (Ripps et al., (1995) Proc. Natl. Acad. Sci, USA, 92: 689-693). For spinal cord injury, the most common models involve contusion injury to rats, either through a calibrated weight drop, or fluid (hydrodynamic) injury. For Huntington's, models involve protection from excitotoxin (NMDA, quinolinic acid, kainic acid, 3-nitro-propionic acid, APMA) lesion to the striatum in rats (Nicholson, L. et al., (1995) Neuroscience 66, 507-521; Beal, M. F. et al., (1993) J. Neuroscience 13, 4181-4192). Recently, a model of transgenic mice overexpressing the human trinucleotide expanded repeat in the huntington gene has also been described (Davies, S. et al., (1997) Cell 90, 537-548). For multiple sclerosis, EAE in mice and rats is induced by immunization with MBP (myelin basic protein), or passive transfer of T cells activated with MBP (Hebr-Katz, R. (1993) Int. Rev. Immunol. 9, 237-285). For Alzheimer's, a relevant murine model is a determination of protection against lesion of the fimbria-fornix in rats (septal lesion), the main nerve bundle supplying the cholinergic innervation of the hippocampus (Borg et al., (1990) Brain Res., 518, 295-298), as well as use of transgenic mice overexpressing the human beta-amyloid gene. For peripheral neuropathies, a relevant model is protection against loss of peripheral nerve conductance caused by chemtherapeutic agents such as taxol, vincristine, and cisplatin in mice and rats (Apfel et al., (1991) Ann. Neurol., 29, 87-90).

The products of the present invention have been found useful in sustaining the half life of hedgehog, and may for example be prepared for therapeutic administration by dissolving in water or acceptable liquid medium. Administration is by either the parenteral, aerosol, or oral route. Fine colloidal suspensions may be prepared for parenteral administration to produce a depot effect, or by the oral route while aerosol formulation may be liquid or dry powder in nature. In the dry, lyophilized state or in solution formulations, the hedgehog protein -polymer conjugates of the present invention should have good storage stability. The thermal stability of conjugated hedgehog protein (data not shown) is advantageous in powder formulation processes that have a dehydration step.

The polymer-hedgehog protein conjugates of the invention may be administered per se as well as in the form of pharmaceutically acceptable esters, salts, and other biologically functional derivatives thereof. In such pharmaceutical and medicament formulations, the hedgehog protein preferably is utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The hedgehog protein is provided in an amount effective to achieve the desired pharmacological effect, as described above, and in a quantity appropriate to achieve the desired daily dose.

The formulations include those suitable for parenteral as well as non-parenteral administration, and specific administration modalities include oral, rectal, buccal, topical, nasal, ophthalmic, subcutaneous, intramuscular, intravenous, transdermal, intrathecal, intra-articular, intra-arterial, sub-arachnoid, bronchial, lymphatic, vaginal, and intra-uterine administration. Formulations suitable for oral, nasal, and parenteral administration are preferred.

When the hedgehog protein is utilized in a formulation comprising a liquid solution, the formulation advantageously may be administered orally or parenterally. When the hedgehog protein is employed in a liquid suspension formulation or as a powder in a biocompatible carrier formulation, the formulation may be advantageously administered orally, rectally, or bronchially.

When the hedgehog protein is utilized directly in the form of a powdered solid, the hedgehog protein may advantageously be administered orally. Alternatively, it may be administered nasally or bronchially, via nebulization of the powder in a carrier gas, to form a gaseous dispersion of the powder which is inspired by the patient from a breathing circuit comprising a suitable nebulizer device.

The formulations comprising the polymer conjugates of the present invention may conveniently be presented in unit dosage forms and may be prepared by any of the methods well known in the art of pharmacy. Such methods generally include the step of bringing the active ingredient(s) into association with a carrier which constitutes one or more accessory ingredients. Typically, the formulations are prepared by uniformly and intimately bringing the active ingredient(s) into association with a liquid carrier, a finely divided solid carrier, or both, and then, if necessary, shaping the product into dosage forms of the desired formulation.

Formulations of the present invention suitable for oral administration may be presented as discrete units such as capsules, cachets, tablets, or lozenges, each containing a predetermined amount of the active ingredient as a powder or granules; or a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered polymer conjugates with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the active conjugate, which preferably is isotonic with the blood of the recipient (e.g., physiological saline solution). Such formulations may include suspending agents and thickening agents or other microparticulate systems which are designed to target the compound to blood components or one or more organs. The formulations may be presented in unit-dose or multi-dose form.

Nasal spray formulations comprise purified aqueous solutions of the active conjugate with preservative agents and isotonic agents.

Formulations for rectal administration may be presented as a suppository with a suitable carrier such as cocoa butter, hydrogenated fats, or hydrogenated fatty carboxylic acid.

Ophthalmic formulations such as eye drops are prepared by a similar method to the nasal spray, except that the pH and isotonic factors are preferably adjusted to match that of the eye.

Topical formulations comprise the conjugates of the invention dissolved or suspended in one or more media, such as mineral oil, petroleum, polyhydroxy alcohols, or other bases used for topical pharmaceutical formulations.

In addition to the aforementioned ingredients, the formulations of this invention may further include one or more accessory ingredient(s) selected from diluents, buffers, flavoring agents, disintegrants, surface active agents, thickeners, lubricants, preservatives (including antioxidants), and the like.

Accordingly, the present invention contemplates the provision of suitable polymers for in vitro stabilization of hedgehog in solution, as a preferred illustrative application of non-therapeutic application. The polymers may be employed for example to increase the thermal stability and enzymic degradation resistance of the hedgehog. Enhancement of the thermal stability characteristic of the hedgehog protein via conjugation in the manner of the present invention provides a means of improving shelf life, room temperature stability, and robustness of research reagents and kits.

The following Examples are provided to illustrate the present invention, and should not be construed as limiting thereof. In particular, it will be understood that the in vivo, animal experiments described herein may be varied, so that other modifications and variations of the basic methodology are possible. These modifications and variations to the Examples are to be regarded as being within the spirit and scope of the invention.

### EXAMPLE 1

### A Survey of Chemistries That Can be Used to Modify Hedgehog with PEG

In an initial survey to understand key variables that could allow us to produce a pegylated version of hedgehog, a diverse series of chemistries were screened that were targeted at free amino groups, thiol groups, and α-amine at the N-terminus of the protein. The studies described below in sections A-C of Example 1 summarize our findings. Examples 2 and 3 describe an alternative strategy in which pegylation was targeted at specific sites at or near the C-terminus of Sonic hedgehog (Shh) that had been engineered into the Shh sequence by site directed mutagenesis. This strategy circumvented unexpected problems that were encountered in these initial studies, providing a highly potent pegylated form of Shh with a specific activity that was 10 times that of unmodified Shh in in vitro studies and exhibited a higher level of activity in animals.

A. Amine Reactive PEGs.

Wild type Shh (corresponding to residues 24-197 in the human gene sequence) was expressed and purified from E. coli as previously described (See copending U.S. Ser. No. 60/067,423).

The wild type was treated with the following commercially available forms: methoxypolyethelene glycol 5000 Da activated with cyanuric acid, p-ntrophenyl carbonate, succinimidyl succinate, and tresylate (Sigma, St. Louis, Mo.); and succinimidyl succinate methoxy-PEGs of varying lengths, 5 kDa, 10 kDa, and 20 kDa (Shearwater Polymers, Inc., Huntsville, Ala.).

Reactions were performed at a variety of pHs ranging from 7.5-8.2 and analyzed for extent of reaction by SDS-PAGE. The reaction products were subjected to size exclusion chromatography on a Superdex® 200 column in PBS and fractions containing unmodified, monosubstituted, disubstituted, and higher modified forms were obtained. These preparations were tested for hedgehog function using the C3H10T1/2 assay discussed above. All of these chemistries resulted in pegylated Shh that was inactive in its ability to promote hedgehog dependent responses in the C3H10T1/2 assay.

We subsequently determined that active hedgehog preparations, albeit with reduced activity (2-3 fold lower than wild type) could be obtained for 5 kDa PEG adducts if the cross-linking chemistry was performed on Shh that was immobilized on cation exchange resins such as POROS SP® (PerSeptive Biosystems, Cambridge, Mass.) or SP-Sepharose® (Pharmacia). In these studies Shh in 20 mM sodium phosphate pH 5.5, 40 mM NaCl was immobilized on SP resin in batch mode for 15 min at room temperature with constant rocking. The resin was washed with 25 mM sodium phosphate pH 5.5, suspended in the same buffer plus 50 mM HEPES pH 8.2, and treated with activated PEG for 2 hours at room temperature with constant rocking. The resins were then washed with 25 mM sodium phosphate pH 5.5 and Shh eluted with PBS pH 7.4 containing 450 mM NaCl. The reaction products were subjected to size exclusion chromatography on a Superdex® 200 column in PBS and fractions containing unmodified, monosubstituted, disubstituted, and higher modified forms were obtained.

In the C3H10T1/2 assay, Shh with the a single 5 kDa PEG added exhibited a level of activity that was 2-3 fold lower than unmodified Shh, while Shh modified with a single 10 or 20 kDa moiety was inactive. Later we discovered that the primary epitope required for binding to the SP matrix is the basic region corresponding to residues 10-15 in the Shh hedgehog sequence. Thus we infer that the effect of the SP matrix was to block modification at sites near the N-terminus of Shh, consistent with the findings described below that PEG modifications at the N-terminus inactivated the protein. The effect of added PEG on clearance was tested in mice by treating mice intravenously with approximately 70 μg of pegylated Shh and then measuring levels remaining in circulation after 7 hours. Values of 0, 0.06, 0.7, 25, 10, and 38 μg/mL of Shh were detected for unmodified, and Shh modified with a single 5 kDa PEG, a single 10 kDa PEG, two 10 kDa PEGs, a single 20 kDa PEG and two 20 kDa PEGs, respectively.

These findings indicated that as expected clearance of Shh could be readily modulated with pegylation and that the rate of clearance correlates with the size and number of added PEGs.

B. Thiol Reactive Chemistries.

Three different chemistries were used to assess the results of targeting the thiol groups on Shh with PEG: (i) a non cleavable activated PEG, methoxy PEG 5000 maleimide Shearwater Polymers, Inc.); (ii) a cleavable version that could be released with reductant, methoxy PEG 5000-orthopyridyl disulfide (Shearwater Polymers, Inc.); and (iii) a reversible linkage that is the resulting product of reaction of Shh with methoxy PEG 5000 aldehyde (Fluka; Buchs, Switzerland).

Modification with these thiol reactive PEGs resulted in addition of a single PEG that was selectively targeted at cysteine-1 at the N-terminus of the protein. Modification with the maleimide PEG resulted in a product that was inactive in its ability to elicit a hedgehog dependent response in C3H10T1/2 cells.

In contrast, the products of the two reversible chemistries had reduced activity compared to the level of activity observed with the unmodified Shh. The low level activity in the orthopyridyl disulfide PEG moiety was expected as disulfide interchange was found to result in slow release of the PEG in serum containing samples from the disulfide linked PEG-Shh adduct. Full activity from the orthopyridyl disulfide PEG modified product was achieved when the sample was treated with reductant prior to measuring its activity in the C3H10T1/2 assay.

Modification with aldehyde PEG results in the formation of a cyclic intermediate between the thiol and a-amino groups on the N-terminal cysteine. This intermediate is a reversible state and with time the PEG is released to generate the free cysteine. Thus the low level activity seen with the reversible PEG adducts is probably due to Shh that was reverted back to its unmodified state during the assay. While the reversible adducts may be useful in providing a form of Shh with longer bioavailability, their reduced activity in the pegylated state means that at a given concentration of Shh only a fraction of the protein is active reducing the available dose.

C. Chemistries Targeted at the N-terminal α-amine of Shh.

Because of the unusually high reactivity of an N-terminal cysteine, we accessed the effects of N-terminal modification on function by mutating the N-terminal cysteine into less reactive amino acid and then tested the effects of N-terminal modification on these variants. Two such mutants C1F Shh (in which the N-terminal cysteine is replaced with a phenylalanine) and C1II Shh (in which the N-terminal cysteine is replaced with two isoleucines) were particularly attractive to test in that they exhibit higher activity than the wild type cysteine 1 Shh when tested in the C3HT101/2 assay. The construction of these variants, purification, and characterization of their structure and function is described in detail in co-pending U.S. Ser. No. 067,423.

We incubated C1F Shh at 0.6 mg/mL in 100 mM sodium phosphate pH 6.0, 150 mM NaCl and 5 mM sodium cyanoborohydride for 16 hr at room temperature in the dark with 10 mg/mL methoxy PEG 20,000 aldehyde (Shearwater Polymers, Inc.). The sample was analyzed for extent of conjugation by SDS-PAGE. The reaction products were subjected to size exclusion chromatography on a Superose® 6 column in 5 mM sodium phosphate pH 5.5, 150 mM NaCl and fractions containing unmodified, and monosubstituted forms were obtained. The pegylated sample was inactive in the C3H10T1/2.

C1II Shh at 1.2 mg/mL in 100 mM sodium phosphate pH 6.0, 150 mM NaCl and 5 mM sodium cyanoborohydride was incubated for 16 hr at room temperature in the dark with 2 mg/mL methoxy PEG 5,000 aldehyde (Fluka). The sample was analyzed for extent of conjugation by SDS-PAGE. The reaction products were subjected to size exclusion chromatography on a Superose® 6 column in 5 mM sodium phosphate pH 5.5, 150 mM NaCl and fractions containing unmodified, and monosubstituted forms were obtained. Like C1F Shh that had been pegylated with PEG 20000 aldehyde, the C1II pegylated sample was also inactive in the C3H10T1/2.

### EXAMPLE 2

### Pegylation of Shh Using a Targeted Chemistry Directed at the C-terminus of Shh

Several observations suggested that pegylation that was targeted at or near the C-terminus of Shh would be a useful strategy for producing active hedgehog: (i) this region in natural Shh is modified with cholesterol and therefore is likely to be available for modification; (ii) we have found that treatment of Shh with thrombin results in cleavage of the protein at a site 3 amino acids from the C-terminus further suggesting that this is an exposed region; (iii) we performed extensive SAR analyses and discovered that the C-terminal 11 amino acids could be deleted without harmful effects on folding or function; and (iv) we have made hedgehog/Ig fusion proteins by attaching an Ig moiety to the C-terminus of hedgehog without harmful effects on folding or function (data not presented here).

Two sites that have been successfully targeted for modification are A169 and G175. These sites were selectively targeted by first mutating the natural amino acids in the human Shh sequence with cysteine residues and then using the thiol groups of these cysteines as targets for cross-linking. Because the wild type Shh sequence has an N-terminal cysteine that is highly reactive and modification at this site with PEG inactivated the protein (Example 1), we replaced Cys1 with the sequence isoleucine-isoleucine by site directed mutagenesis. This change in sequence has the added benefit of not only protecting the N-terminus from modification, but it results in a hedgehog product that is 10 times more potent than wild type Shh. While the following examples are limited to studies with this variant, we expect that any mutation targeted at Cys1 or modification of this cysteine can be used in place of the Ile-Ile substitution.

A. Construction of the C1II/A169C Mutant

N-terminal cysteine mutants of soluble huShh were made by unique site elimination mutagenesis using a Pharmacia kit following the manufacturer's recommended protocol and employing the mutagenic oligonucleotide design principles described below. In designing the mutagenic primers, if a desired mutation did not produce a restriction site change, a silent mutation producing a restriction site change was introduced into an adjacent codon to facilitate identification of mutant clones following mutagenesis. To avoid aberrant codon usage, substituted codons were selected from those occurring at least once elsewhere in the huShh cDNA sequence. As a first step in the mutagenisis we inserted the C1II mutation into p6H-Shh to form a C1II Shh template which was then further mutagenized to add the specific point mutations designed to target the pegylation.

Construction of the C1II Shh template pEAG872 was carried out as follows: The 584 bp NcoI-XhoI restriction fragment carrying the his-tagged wild-type Shh N-terminal domain from p6H-SHH was subcloned into the pUC-derived cloning vector pNN05 to construct the plasmid pEAG649. p6H-SHH was provided by David Bumcrot (Ontogeny, Inc.) and it carries human sonic hedgehog coding sequences starting at amino acid 1 of the mature Shh protein and extending to amino acid 175 (the N-terminal domain), followed by tandem termination codons. The DNA was cloned as an NcoI-XhoI fragment in pET11d so that the Shh coding sequences were downstream of sequences encoding 6 consecutive histidine residues and a recognition sequence (DDDDK) for the restriction protease enterokinase (Marti et al., Nature 375: 322-325, 1995). Presence of the introduced restriction site change was reconfirmed in the expression vector. The C1II mutation was introduced into pEAG649 by two cloning steps. First, we generated an intermediate cysteine-1 mutant C1F, plasmid pEAG837 using the following mutagenic primer: 5′ GGC GAT GAC GAT GAC AAA TTC GGA CCG GGC AGG GGG TTC 3′ (SEQ ID NO: 27), which introduces an ApoI site. The mutation was confirmed by DNA sequencing through a 180 bp NcoI-BglII restriction fragment carrying the mutant Shh proteins' N-termini in plasmid pEAG837. Second, the following mutagenic primer was used on plasmid pEAG837 to make C1II: 5′ GCG GCG ATG ACG ATG ACA AAA TCA TCG GAC CGG GCA GGG GGT TCG GG 3′ (SEQ ID NO: 28), which removes an ApoI site to make pEAG872. The mutation was confirmed by DNA sequencing through a 0.59 kb NcoI-XhoI restriction fragment carrying the mutant Shh.

We next introduced the A169C mutation into the Shh C1II template to make the soluble human Shh mutant C1II/A169C by replacing the dispensable C-terminal residue A169 with cysteine. The following mutagenic primer 5′ GAG TCA TCA GCC TCC CGA TTT TGC GCA CAC CGA GTT CTC TGC TTT CAC C 3′ (SEQ ID NO: 29) was used on C1II Shh template pEAG872 to add the mutation. The primer also resulted in the formation of an FspI site. Vector pEAG872 with the FspI site was designated vector pSYS049. The C1II/A169C mutations were confirmed by DNA sequencing through a 0.59 kb NcoI-XhoI restriction fragment. The expression vector pSYS050 was constructed by subcloning the NcoI-XhoI fragment from pSYS049 into the phosphatase-treated 5.64 kb XhoI-NcoI pET11d vector backbone of p6H-SHH.

The expression vector pSYS050 was transformed into competent E. coli BL21(DE3)pLysS, colonies were selected, induced, and screened for Shh expression as described in U.S. Ser. No. 60/067,423.

B. Purification of the C1II/A169C Mutant.

Bacterial pellets from cells expressing ShhII 169C at 4-5% of the total protein were thawed, resuspended in lysis buffer (25 mM sodium phosphate pH 8, 150 mM NaCl, 0.2 mM PMSF, 0.5 mM DTT) at a ratio of 1:4 (w/v) and lysed by two passes through a Gaulin press (APV Rannie, Copenhagen, Denmark) at 12,000 pounds per square inch. All subsequent purification steps were performed at 2-8° C. unless indicated otherwise. The homogenate was centrifuged at 19,000 g for 60 min and NaCl from a 5M stock was added to the resulting lysate to produce a final NaCl concentration of 300 mM.

This material was loaded onto a Ni-NTA agarose (Qiagen, Santa Clara, Calif.) column (4 g E. coli wet weight/mL resin) previously equilibrated with 25 mM sodium phosphate pH 8, 0.3 M NaCl, 0.5 mM DTT. The column was washed with 1 column volumes (CV) of the same buffer, then 3 CV of the same buffer plus 20 mM imidazole (diluted from a 1M stock solution at pH 7) and 100 mM NaCl, and histag-Shh eluted with 3 CV 25 mM sodium phosphate pH 8, 400 mM NaCl, 200 mM imidazole, 0.5 mM DTT. Nine 0.2 CV fractions were collected and the protein content in each fraction was determined by absorbance at 280 nm.

The peak fractions were pooled, diluted with 3 volumes of 100 mM MES pH 5.0 and loaded onto an SP Sepharose® Fast Flow (Pharmacia, Piscataway, N.J.) (15 mg protein/mL resin) previously equilibrated with 25 mM sodium phosphate pH 5.5, 150 mM NaCl. The column was washed with 3 CV of equilibration buffer, then with 1 CV of 25 mM sodium phosphate pH 5.5, 300 mM NaCl, 0.5 mM DTT, and histag-Shh was eluted with 800 mM NaCl in the same buffer. Elution fractions were analyzed for absorbance at 280 nm and by SDS-PAGE. Typically, we recovered about 1-2 g of his-tagged Shh from 0.5 kg of bacterial paste (wet weight). The product was filtered through 0.2 μm filter, aliquoted and stored at −70° C. The histagged Shh was about 95% pure as determined by SDS-PAGE.

To cleave off the six histidine tag, ShhII 169C was diluted with 0.5 volume of 50 mM sodium phosphate pH 8.0, and enterokinase (Biozyme, San Diego, Calif.) was incubated with the histag-Shh at an enzyme:Shh ratio of 1:500 (w/w) for 2 h at 28° C. Uncleaved histag-Shh and free histag were removed by passing the digest through a second Ni-NTA agarose column (20 mg Shh/mL resin). Prior to loading, imidazole (1 M stock solution at pH 7) was added to the digest to give final concentrations of 20 mM. The column was washed with 1 CV of 25 mM sodium phosphate pH 8, 400 mM NaCl, 200 mM imidazole, 0.5 mM DTT and the wash fraction was pooled with the flow through.

The Ni agarose unbound fraction was diluted with 2 volumes of 60 mM MES pH 5.0 and this material was loaded onto a second SP Sepharose® Fast Flow column (20 mg protein/ml resin) equilibrated with 5 mM sodium phosphate pH 5.5, 150 mM NaCl, 0.2 mM DTT. The column was washed with 3 CV of equilibration buffer and 1 CV of the same buffer containing 300 mM NaCl. Shh was eluted with 5 mM sodium phosphate pH 5.5, 800 mM NaCl, 0.2 mM DTT. The resulting Shh was >98% pure as characterized by SDS-PAGE. After removal of the histag and subsequent purification, the purified Shh was shown to have the expected mass as measured by ESI-MS. The product was filtered through 0.2 μm filter, aliquoted and stored at −70° C.

C. Modification of Shh II 169C with PEG Maleimide.

For modification with methoxy polyethylene glycol 20,000-maleimide (M-MAL-20,000; Shearwater Polymers, Inc., Huntsville, Ala.), we diluted 3.45 mL of purified C1II/A169C at 18 mg/mL in 5 mM Na₂HPO₄ pH 5.5, 800 mM NaCl, 0.2 mM DTT with 6 mL of 50 mM MES pH 6.5 and then added 1 mL of 80 mg/mL PEG maleimide. These conditions were selected such that the molar amount of maleimide added equaled the amounts of thiols in the reaction contributed by reactants where DTT equals 2 per mole and Shh equals 1 per mole.

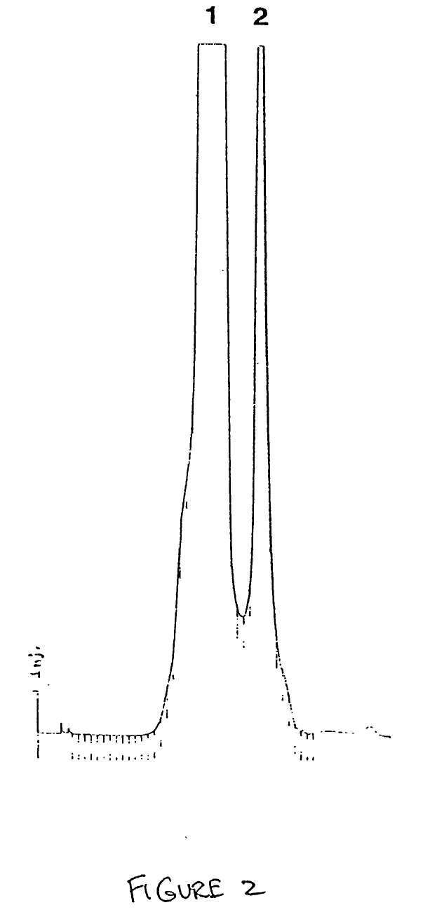

The sample was incubated for 90 min at room temperature in the dark. At this time additional DTT was added to 0.5 mM and the sample incubated further for an additional hour at room temperature. The sample was filtered and then stored overnight at 4° C. Typically under these conditions, about 90% of the Shh was modified with a single PEG moiety. The pegylated Shh II was purified from non reacted product by chromatography on a Superose® 6 FPLC sizing column (Pharmacia) with 5 mM sodium phosphate pH 5.5, 150 mM NaCl as the mobile phase. Elution fractions were analyzed for protein content by absorbance at 280 nm and SDS-PAGE. Fractions containing pegylated protein were pooled, filtered through 0.2 μm filter, aliquoted and stored at −70° C. The concentration of Shh is reported in Shh equivalents as the PEG moiety did not contribute to absorbance at 280 nm. FIG. 2 shows a chromatogram illustrating the fractionation of PEG-modified and unmodified Shh II A169C on a Superose 6 column. PEG-modified and unmodified Shh are well resolved by the sizing chromatography step, with the modified product eluting first, consistent with its larger size.

D. Biochemical Characterization of PEGylated Shh II.

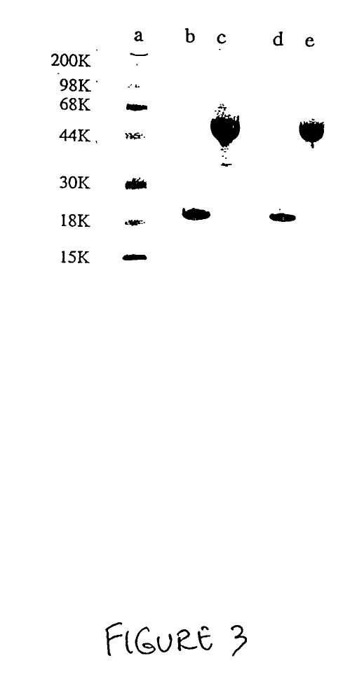

Samples were analyzed for extent of modification by SDS-PAGE (FIG. 3). Addition of a single PEG resulted in a shift in the apparent mass of Shh from 20 kDa to 55 kDa which was readily apparent upon analysis. In the pegylated sample there was no evidence of unmodified Shh II 169C nor of higher mass forms resulting from the presence of additional PEG groups. The presence of a single PEG was verified by MALDI-TOF mass spectrometry) on a Voyager-DE™ STR (PerSeptive Biosystems, Framingham, Mass.) mass spectrometer. Unmodified Shh II 169 had an observed mass of 19850 Da minus PEG and of 40460 Da after pegylation.

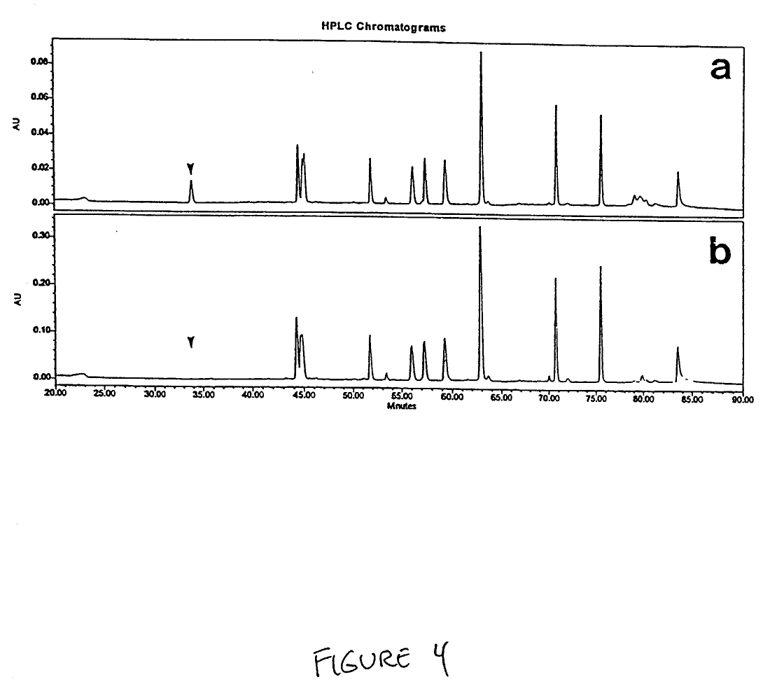

The specificity of the pegylation reaction was evaluated by peptide mapping. Alkylated protein (0.4 mg/mL in 1 M guanidine hydrochloride, 20 mM Na₂HPO₄ pH 6.0) was digested with endo Lys-C (Wako Pure Chemical Industries, Ltd.) at a 1:20 ratio. The digestion was conducted at room temperature for 30 h. The reaction was stopped by acidification with 5 μL of 25% trifluoroacetic acid. The digest was analyzed on a Waters 2690 Separation Module with a Model 996 photodiode array detector. Prior to injection, solid guanidine hydrochloride was added into the digest to a concentration of 6 M to dissolve insoluble material. A reverse phase Vydac® C₁₈ (2.1 mm internal diameter×250 mm) column was used for separation, with a 90 min gradient of 0.1% trifluoroacetic acid/water and 0.1% trifluoroacetic acid/acetonitrile at a flow rate of 0.2 mL/min. Individual peaks were collected manually for mass analysis. Results from the analysis are shown in FIG. 4. All of the predicted peptides from the endoproteinase Lys-C digest of Shh II 169C have been identified by mass spectrometry and of these, only the peptide that contains C169 (denoted with arrowhead) was altered by the modification as evident by its disappearance from the map. The mapping data therefore indicate that the PEG moiety is specifically attached to this peptide.

E. Assessing the Potency of Pegylated Shh.

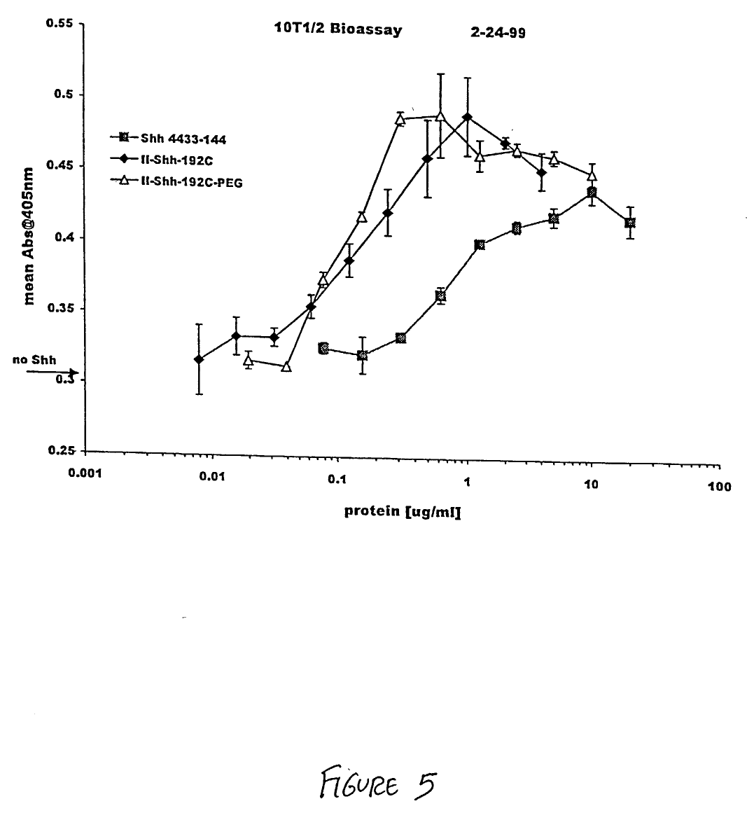

The activity of Shh II samples was tested on C3H10T1/2 cells using the procedures described below. The murine pluripotent mesenchymal cell line C3H10T1/2 was obtained from the American Type Culture Collection (ATCC). C3H10T1/2 cells were maintained in DMEM medium containing 10% FBS. For assessing hedgehog activity, C3H10T1/2 cells were plated in 96-well plates at 5000 cells/well and 24 h later purified hedgehog protein was added and the cells incubated for a further 5 d. Cells were then lysed and assayed for alkaline phosphatase (AP) activity using the chromogenic substrate p-nitrophenylphosphate and read at 405 nm. AP is a marker for differentiation into the osteoblast lineage. Typical dose responses were in the range of 0.1 to 10 g/L. The specific activity of the pegylated Shh II 169C (150-200 ng/mL) was indistinguishable from that of the unmodified Shh II 169C, indicating that the modification had not effected function (FIG. 5). In the same assay wild type Shh had a specific activity of 1-2 μg/mL and therefore the pegylated Shh is about 10 times as active as wild type Shh in its ability to elicit a hedgehog-dependent response.

Shh II 169 C was also PEGylated with: (i) a 5K PEG-maleimide moiety that was purchased from Fluka, Inc. (Cat. No. 63187, Ronkonkoma, N.Y.) following the same protocol described for modification with 20K PEG maleimide; (ii) a 5K PEG-vinylsulfone moiety that was purchased from Fluka, Inc. (Cat. No. 95066, Ronkonkoma, N.Y.); and (iii) a 5 K iodoacetamide PEG (Shearwater Polymers, Inc.). In all cases modification with the 5K PEG moieties was highly specific for the C-terminal cysteine C169 as determined by peptide mapping and the activities of the modified Shh II 169 adducts in the C3H10T1/2 assay were indistinguishable from that of the unmodified Shh II 169C. Based on these findings we envision that any chemistry that can be selectively targeted at the thiol group on surface Cys residues can be used to modify the Shh II 169C mutant. As part of this analysis, Shh II 169C was also PEGylated with a 20K PEG-vinylsulfone moiety that was purchased from Shearwater polymers, Inc. This product exhibited similar properties as the 20 K maleimide adduct.

To understand what effect pegylation of Shh might have on function we treated Ptc-LacZ mice with wt and Shh that had been pegylated with the 20 K PEG maleimide. Transgenic mice containing the ptc promotor fused to the LacZ gene generated by homologous recombination were generated as previously described (Goodrich et al., 1997) and were obtained from Matt Scott (Stanford U.). The Ptc-LacZ mouse provides a simple animal model to assess the potency of ShhN constructs in vivo. Hh-induced increases in β-gal activity in these animals, which can be used as a marker for ptc-1 containing cells and hence Hh-responsive tissue, and can be readily quantified.

In these studies, Ptc-LacZ mice were given by subcutaneous administration either 3 or 10 mg/kg of pegylated Shh or 10 or 30 mg of unmodified ShhN twice a day for three days. Mice were killed and their lungs, kidneys, and hearts were removed. The tissues were homogenized and the resulting lysates assayed for β-galactosidase activity. Both wt ShhN and pegylated induced an increase in β-gal activity in all organs tested after given subcutaneous administration. However, pegylated Shh was substantially more potent that unmodified Shh. Similar levels of β-gal induction were observed with a 10-fold lower dose of the pegylated product, indicating a substantial improvement in the efficacy of the modified Shh.

F. Assessing the Pharmacodynamics (PK) of Pegylated Shh in Rats.

Pegylated ShhII 169C (modified with 20 K PEG maleimide) was evaluated for PK in rats following intravenous (i.v.) or subcutaneous (s.c.) administration. In these studies 3 rats/group were treated either with 3 mg/Kg pegylated Shh s.c. or with 1 mg/Kg pegylated Shh i.v. and bleeds taken after 0.08, 0.25, 0.5, 1, 2, 4, 6, 8, 24, and 48 hr following administration. Levels of Shh in blood serum were determined by ELISA using anti-hedgehog antibodies as probes.

Surprisingly, after i.v. administration, there was only a slight (i.e., less than 2-fold) decrease in the rate at which pegylated Shh compared to a non modified Shh control was cleared from circulation. Maximum levels of about 29-35 μg/mL in blood were seen at 0.08 hr after administration. After 1 hr levels dropped to 10-15 μg/mL, then to 4-5 μg/mL after 2 hrs, to 1.5-2 μg/mL after 4 hrs, to 0.4-0.6 μg/mL after 6 hrs, to 0.2-0.3 μg/mL after 8 hrs, and to 0.007-0.016 μg/mL after 24 hrs. The data indicate that loss of Shh from blood following i.v. administration is probably the result of specific sequences on the Shh protein that govern its clearance and that modification at the A169C site has little effect on normal clearance of Shh from circulation. We calculated distribution and elimination half lives of 0.9 h and 3.2 h, respectively, from the levels of pegylated Sonic hedgehog in the blood.

In contrast, the bioavailability of pegylated Shh following subcutaneous administration was greatly improved over that of unmodified Shh, which therefore is likely to account for its improved efficacy in models. After treatment with pegylated Shh, two of the animals showed similar profiles with levels of 2 and 13, 31 and 72, 59 and 94, 117 and 114, 223 and 216, 543 and 463, 614 and 478, 466 and 353, and 39 ng/mL after 0.08, 0.25, 0.5, 1, 2, 4, 6, 8, 24, and 48 hrs, respectively. Data for unmodified Shh following s.c. administration peaked after 0.25-1 hr with levels of 300 and 436 ng/mL in two animals that were tested, but levels were down 10 fold after 6 hr, and down 10-fold further at 2 ng/mL 24 hr following administration. Therefore, with pegylation high levels were maintained from 2-24 hrs whereas with wild type protein peak levels were maintained for less than 6 hrs. Based on calculated areas under the curve for the pegylated Shh clearance data, we estimate that bioavailability of the pegylated protein was about 30% of the total dose. For one of the animals treated with pegylated Shh, the profile looked more like what would be expected for an intramuscular administration with levels of 0.29, 1.4, 1.8, 3.0, 2.8, 1.7, 0.94, 0.68, 0.35, and 0.04 μg/mL present after 0.08, 0.25, 0.5, 1, 2, 4, 6, 8, 24, and 48 hrs, respectively. Like the other two animals in the group, significant concentrations of pegylated Shh were maintained for 24 hr. Since activity data in the C3H10T1/2 cell assay and in animals revealed that efficacy with Shh treatment requires a continuous treatment with the protein throughout the duration of the studies, the improved delivery of the pegylated Shh following s.c. administration is likely to account for its improved efficacy in animals.

### EXAMPLE 3

### Preparation of Pegylated Shh C175

A. Construction of the C1II/G175C Shh Mutant.

The soluble His-tagged human Shh mutant C1II/G175C (with cysteine substituted for the C-terminal residue G175) was made by unique site elimination mutagenesis using a Pharmacia kit following the manufacturer's recommended protocol. The following mutagenic primer 5′ CCA CCA ATC TCA AAG CTC TCG AGC TAT CAG CAG CCT CCC GAT TTG GCC GC 3′ (SEQ ID NO: 30) was used on C24II SHH template pEAG872 (described above) to remove a HinfI site to make pSYS045. The C1II/G175C mutations were confirmed by DNA sequencing through a 0.59 kb NcoI-XhoI restriction fragment.

The expression vector pSYS046 was constructed by subcloning the NcoI-XhoI fragment from pSYS045 into the phosphatase treated 5.64 kb XhoI-NcoI pET11d vector backbone of p6H-SHH (described above). Presence of the introduced restriction site change was reconfirmed in the expression vector. The expression vector was transformed into competent BL21(DE3)pLysS cells, colonies were selected, induced, and screened for Shh expression as previously described.

B. Modification of Shh II 175C with PEG Maleimide.

Shh C1II/G175C was purified from bacterial cell pellets using the method described in Example 2 for the purification of Shh II 169C. For modification with methoxy polyethylene glycol 20,000-maleimide (M-MAL-20,000; Shearwater Polymers, Inc.), 50 μL of purified C1II/G175C Shh at 1.9 mg/mL in 5 mM Na₂HPO₄ pH 5.5, 800 mM NaCl, 0.5 mM DTT was diluted with 125 μL of 70 mM MES pH 6.5, 150 mM NaCl and to this 10 μL of 140 mg/mL PEG maleimide was added.

These conditions were selected such that the molar amount of maleimide added equaled the amounts of thiols in the reaction contributed by reactants where DTT equals 2 per mole and Shh equals 1 per mole. The sample was incubated for 90 min at room temperature in the dark. At this time additional DTT was added to 0.1 mM and the sample incubated further for an additional hour at room temperature. The sample was filtered and frozen at −70° C. Samples were analyzed for extent of modification by SDS-PAGE. Addition of a single PEG resulted in a shift in the apparent mass of Shh from 20 kDa to 55 kDa which was readily apparent upon analysis.

The activity of pegylated Shh C1II/G175C was tested on C3H10T1/2 cells using the procedures described below and was indistinguishable from unmodified Shh C1II/G175C. Both the pegylated and unmodified Shh C1II/G175C exhibited IC50 values in this assay of 150-200 ng/mL.

These findings verify that the A169C mutation is not unique in its ability to serve as a site for attachment of PEG without perturbing hedgehog function and that other positions in the Shh sequence such as G175 can be used equally as well as a target for pegylation We expect that any residue in Shh can be substituted for which meets the following criteria: (i) The thiol group on the substituted amino acid is exposed and assessable for modification; (ii) The amino acid mutation itself does not perturb function; and (iii) The addition of the PEG moiety itself does not alter function. The available crystal structure for Shh should aid in the selection of possible residues that can be targeted.

### EXAMPLE 4

### Preparation of Pegylated Desert Hedgehog (Dhh)

A. Construction of the C1II/A170C Dhh Mutant.

The 0.62 kb XhoI-ApaI fragment carrying an N-terminal human DHH cDNA from a plasmid provided by D. Bumcrot (Ontogeny, Inc., Cambridge, Mass.) was subcloned into pBluescriptII-SK+ to make the plasmid pEAG666. The mutagenic primer 5′ GGC CCC CGG CCC GGA CCG CAG CTC TGG GCT G 3′ (SEQ ID NO: 31), which adds a 5′ RsrII site, and the mutagenic primer 5′ GGG TAC CGG GCC CTC CTC GAG TCA TCA GCC GCC CGC CCG CAC CGC CAG TGA G 3′ (SEQ ID NO: 32), which removes a 3′ RsrII site and adds a 3′ XhoI site, were used on template pEAG666 to make pEAG679. The mutations were confirmed by DNA sequencing through a 532 bp RsrII-XhoI fragment of pEAG679.

pEAG683, the E. coli expression vector for his-tagged wild type huDhh was made by subcloning the 532 bp RsrII-XhoI fragment from pEAG679 into the phosphatase-treated 5.70 kb XhoI-RsrII vector backbone from p6H-SHH (described above). Presence of Dhh-specific sequences was confirmed by restriction enzyme digestion of the expression vector. The expression vector was transformed into competent BL21(DE3)pLysS cells, colonies were selected, induced, and screened for DHH expression as previously described.

The 526 bp NcoI-XhoI fragment from pEAG683 was subcloned into the pUC-derived cloning vector pNN05, to make plasmid pEAG749. The C1II mutation was made by using mutagenic primer 5′ CAG CGG CGA TGA CGA TGA CAA AAT CAT CGG CCC GGG CCG GGG GCC GGT TG 3′ (SEQ ID NO:33), which adds a SmaI site, on template pEAG749 to make pEAG873. The C1II mutation was confirmed by DNA sequencing through the 593 bp NcoI-XhoI fragment of pEAG873.

The Dhh mutant C1II/A170C was made using mutagenic primer 5′ GTC ATC AGC CGC CCG CCC GTA CGC ACA GTG AGT TAT CAG CTT TGA C 3′ (SEQ ID NO: 34), which adds an RsaI site, on template pEAG873 to make pEAG949. The mutation was confirmed by DNA sequencing through the through the 593 bp NcoI-XhoI fragment of pEAG949.

pEAG952, the E. coli expression vector for his-tagged huDhh mutant C1II/A170C was made by subcloning the 593 bp NcoI-XhoI fragment from pEAG949 into the phosphatase-treated 5.64 kb XhoI-NcoI pET11d vector backbone of p6H-SHH. Presence of the introduced restriction site changes were reconfirmed in the expression vector. The expression vector was transformed into competent BL21(DE3)pLysS cells, colonies were selected, induced, and screened for DHH expression as previously described.

B. Modification of Dhh II 170C with PEG Maleimide.

Dhh II170C was purified from bacterial cell pellets using the method described in Example 2 for the purification of Shh II 169C. For modification with methoxy polyethylene glycol 20,000-maleimide (M-MAL-20,000; Shearwater Polymers, Inc.), 7.5 mL of purified C1II/A170C Dhh at 10 mg/mL in 5 mM Na₂HPO₄pH 5.5, 800 mM NaCl, 0.2 mM DTT was diluted with 1 mL of 0.5 M MES pH 6.5 and to this 1.5 mL of 110 mg/mL PEG maleimide was added. These conditions were selected such that the molar amount of maleimide added equaled the amounts of thiols in the reaction contributed by reactants where DTT equals 2 per mole and Dhh equals 1 per mole. The sample was incubated for 90 min at room temperature in the dark. At this time additional DTT was added to 0.1 mM and the sample incubated further for an additional hour at room temperature. The sample was filtered and frozen at −70° C.

The pegylated Dhh II was purified from non reacted product by chromatography on a Superose® 6 FPLC sizing column (Pharmacia) with 5 mM sodium phosphate pH 5.5, 150 mM NaCl as the mobile. Elution fractions were analyzed for protein content by absorbance at 280 nm and SDS-PAGE. Fractions containing pegylated were pooled and the concentration is reported in Dhh equivalents as the PEG moiety did not contribute to absorbance at 280 nm.

C. Biochemical Characterization of PEGylated Dhh II.

Samples were analyzed for extent of modification by SDS-PAGE. Addition of a single PEG resulted in a shift in the apparent mass of Dhh from 20 kDa to 55 kDa which was readily apparent upon analysis. In the pegylated sample there was no evidence of unmodified Dhh II 170C nor of higher mass forms resulting from the presence of additional PEG groups. The activity of Dhh II samples was tested on TM3 cells using the procedures described below. The TM3 cell line was obtained from ATCC and transfected with a luciferase reporter gene that had engineered behind the gli promotor (the cell line was a gift of D. Israel at Ontogeny, Inc., Cambridge, Mass.). For assessing hedgehog activity, the TM3 gli luciferase cells were plated in 96-well plates and 24 h later purified hedgehog protein was added and the cells incubated for a further 24 hr. Cells were then lyzed and assayed for luciferase activity on a Tropix TR717 microplate luminometer. The specific activity of the pegylated Dhh II 169C was similar to that of the unmodified Dhh II 170C, indicating that the modification had not effected function.

### EXAMPLE 5

### Preparation of Truncated Forms of Pegylated Shh.

A truncated N-9 version of pegylated Shh II C169 was generated by treating the modified protein with bovine plasmin (Sigma, St. Louis, Mo.) in 50 MM Tris-HCl pH 7.4 for 3 h at 20° C. at a ShhN:enzyme ratio of 10:1 (w/w). Plasmin was removed by passing the digest through an ovoinhibitor (Pierce, Rockland, Ill.) column in the same buffer. The truncated Shh was tested for function in the C3H10T1/2 assay and was inactive at eliciting a hedgehog response. The truncated Shh was also tested for its ability to compete with wild type Shh for eliciting a hedgehog dependent response on C3H10T1/2 cells and it was functional as a Shh antagonist. As described in U.S. Ser. No. 60/106,703, we previously demonstrated that truncated versions of Shh or other alterations at the N-terminus of the protein that blocked its ability to elicit a response in the C3H10T1/2 assay but did not block patched-1 binding, such as the N-9 version of the protein, could be used as functional antagonists of Shh. While we have investigated many different classes of antagonists, our present ability to produce a pegylated antagonist suggests that our previous strategies for preparing antagonists can be adopted to prepare a host of pegylated hedgehog antagonists. The addition of PEG to such molecules should likewise improve their bioavailability in animals.

### EXAMPLE 6

### Identification of Other Sites in Hedgehog That can be Targeted for Pegylation.

While sites near the C-terminus of hedgehog proteins were selected for targeting pegylation based on biochemical observations that we made, the modest increase in IV half life observed in animals with these pegylated products suggested that it would be beneficial to add additional PEG moieties at other sites on the hedgehog surface. To select potential sites for modification we used the crystal structure of murine Shh (Tanaka Hall, T. M. et al. 1995, Nature 378, 212-216) to identify amino acid residues whose side chains might be accessible to modification with PEG groups and mutated these residues to cysteines. While many amino acid residues fit this criteria, the following eleven amino acid residues were selected for our initial analysis: N27, N46, Y57, N68, K82, N92, S112, E113, S133, G146, and S154. We envision that other sites may be used and that the same strategy we used to generate the eleven mutants indicated could be used on these other sites. We also envision that the corresponding sites that are identified as beneficial in the case of Shh can be mutated in Dhh and Ihh.

Point mutations were incorporated into Shh DNA by unique site elimination mutagenesis using a Pharmacia kit following the manufacturer's recommended protocol and employing the mutagenic oligonucleotide design principles described above. The following mutagenic primers were used on the C1II A169C Shh template and the resulting restriction sites that were added as a result of the mutagenesis steps are listed below. (1) N27C: 5′ GGC GCC TAG GGT CTT CTC AGC CAC ACA GGG GAT AAA CTG CTT GTA GGC 3 (SEQ ID NO: 35)′, which introduces a new DdeI site; (2) N46C: 5′GAGTTC CTT AAA TCG CTC GGA GCA CCT GGA GAT CTT CCC TTC 3′ (SEQ ID NO: 36), which introduces a new Bsp12861 site; (3) Y57C: 5′ CAT CCT TAA ATA TGA TGT CCG GGT TGC AAT TGG GGG TGA GTT CCT TAA ATC G 3′ (SEQ ID NO:37), which introduces a new NciI site; (4) N68C: 5′ CAT CAG CCT GTC CGC TCC GGT ACA TTC TTC ATC CTT AAA TAT GAT GTC 3 (SEQ ID NO: 38)′, which introduces a new RsaI site; (5) K82C: 5′ GAG ATG GCC AAA GCG TTT AAG CAG TCC TTA CAC CTC TGA GTC 3′ (SEQ ID NO: 39), which introduces a new MseI site; (6) N92C: 5′ GTT TCA CTC CTG GCC ACT GAC ACA TCA CCG AGA TGG CCA AAG 3′ (SEQ ID NO: 40), which removes a BsrI site; (7) S112C: 5′ GTA GTG CAG AGA CTC CTC GCA GTG GTG GCC ATC TTC G 3′ (SEQ ID NO: 41), which removes a DdeI site; (8) S133C: 5′ CCA GCA TGC CGT ACT TGC AGC GAT CGC GGT CAG ACG TGG 3′ (SEQ ID NO: 42), which introduces a new PvuI site; (9) S 154C: 5′ CAG TGG ATA TGT GCC TTG CAC TCG TAG TAC ACC CAG TC 3′ (SEQ ID NO: 43), which removes a HinfI site, (10) E113C: 5′ GTA GTG CAG AGA CTC GCA TGA GTG GTG GCC ATC TTC G 3′ (SEQ ID NO: 44), which removes a DdeI site, and (11) G146C: 5′ GTA CAC CCA GTC GAA GCA GGC CTC CAC CGC CAG GC 3′ (SEQ ID NO: 45), which removes an MspI site. The fidelity of the resulting constructs was verified by DNA sequencing. Expression vectors were generated by subcloning the DNA inserts into phosphatase-treated, 5.64 kb XhoI-NcoI pET11d vector backbone of p6H-SHH. The presence of the introduced restriction site was reconfirmed in the expression vector. The expression vectors were transformed into competent BL21(DE3)pLysS cells, colonies selected, induced, and screened for SHH expression as previously described. The Shh mutants were purified following the same strategy disclosed in EXAMPLE 2 for purification of ShhII 169C and the resulting proteins were pegylated also as described.

We also made a mutant where we put back A192C to alanine in order to get back to a single site of pegylation. This form is suitable for expression in E. coli and one may readily shorten its C-terminus via genetic truncations to minimize potential sites of antigenicity. Briefly, the expression vector for his-tagged SHH variant C24II/N91C was constructed by subcloning the 359 bp NcoI-BstEII fragment from pEAG1088 (C24II/N91C/A192C cDNA) and the 228 bp BstEII-XhoI fragment from pEAG872 (C24II cDNA) into the 5.64 kb XhoI-NcoI vector fragment from p6H-SHH. The SHH cDNA NcoI-XhoI insert in the resultant plasmid, called pEAG1270, was confirmed by DNA sequencing. E. coli strain BL21(DE3)pLysS transformed with pEAG1270 were tested for SHH expression by IPTG induction as described herein for pAND020 (C24II/N91C/A192C expression vector). Expression of C24II/N91C was equivalent to that of C24II/N91C/A192C (assayed by SDS-PAGE by Coomassie stain and by Western blot analysis with anti-SHH polyclonal r1200 Ab). C-terminal genetic truncations are made by site-directed mutagenesis by insertion of a new TGA termination codon immediately after the appropriate C-terminal residue's codon. We use the Amersham-Pharmacia Biotech Unique Site Elimination kit for doing site-directed mutagenesis, following the manufacturer's recommended protocol.

Under certain circumstances, it may be advantageous to remove the histidine tag and directly express one or more of the N-terminal hedgehog forms described herein. A preferred host would be E. coli (although other hosts may be used), particularly if large amounts of protein are needed for large-scale and/or commercial production. We removed the histidine tag from mutant 20 (see Table 1), enabling it be expressed in E. coli. Briefly, the his tag was removed and wildtype C24 SHH expressed in E. coli (the cDNA encodes MCGPGRGF . . . at the SHH N-terminus) via a plambda promoter-driven expression construct.

We note that expression of the C24II (i.e., isoleucine-isoleucine) N-terminus in mammalian cells cannot be correctly directed by the native SHH signal sequence since signal cleavage is aberrant, resulting in inactive forms of SHH. A variant of C24II soluble SHH using the signal sequence of pepsinogen A has been constructed and we expect that we can obtain mammalian cell expression of this variant, since it is known that rhesus monkey pepsinogen A has an II N-terminus.

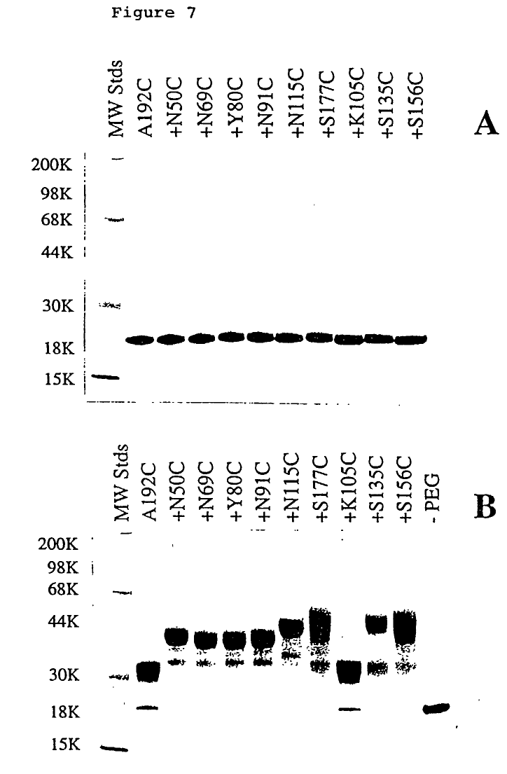

When analyzed by SDS-PAGE we observed that ten of the eleven constructs were pegylated at the newly engineered sites while one the mutants was not modified (FIG. 7 shows modification reactions for 9 of the 11 mutants). The proteins were next tested for function in the C3H10T1/2 assay and three of them retained full activity. Similar results were obtained when the pegylated products were tested for their ability to bind Ptc-1. The results from these analyses are summarized below in Table I. A similar set of activity data was obtained for the mutants following modification with 20K PEG maleimide in place of the 5 K PEG maleimide moiety except that mutant 17, 24, and 44 exhibited even a greater reduction in activity after modification with 20 K PEG than was seen after modification with the 5 K PEG.

TABLE I
Shh-II 192C Double Cys Mutants-Ptc-1 binding data

| | IC₅₀ in C3H10T1/2 | | | | | |
|---|---|---|---|---|---|---|
| Protein | assay in μg/mL | | | Ptc binding (nM) | | |
| Shh variant | unmodified | 5 K PEG | NEM | unmodified | 5 K PEG | NEM |
| wildtype | 2 | >20 | 1 | 2.4 | 42 | 1.4 |
| C1II/169C | 0.3 | 0.3 | 0.2 | 1.1 | 1 | 0.4 |
| mutant 17 +N27C | 0.4 | 4 | 0.3 | 1.3 | 32 | 0.3 |
| mutant 18 +N46C | 0.4 | 0.3 | 0.2 | ND | 2 | 2 |
| mutant 19 +Y57C | 0.2 | 0.2 | 0.2 | ND | 3 | 1 |
| mutant 20 +N68C | 0.3 | 0.2 | 0.2 | 3.0 | 3 | 2 |
| mutant 21 +N92C | 1.5 | >20 | 2 | ND | 35 | 0.3 |
| mutant 22 +S154C | 1 | 5 | 2 | 3.9 | 85 | 8.3 |
| mutant 23 +K82C* | 0.2 | - | - | - | - | - |
| mutant 24 +S112C | 0.4 | 0.6 | 0.2 | ND | 8 | 0.6 |
| mutant 25 +S133C | 0.4 | 2 | 0.2 | 3.7 | 33 | 0.4 |
| mutant 44 +E113C | 1 | 0.6 | ND | 2.3 | 12 | 0.5 |
| mutant 45 +G146C | 0.3 | 0.5 | ND | ND | ND | ND |

*modification chemistries failed
ND, notdetermined

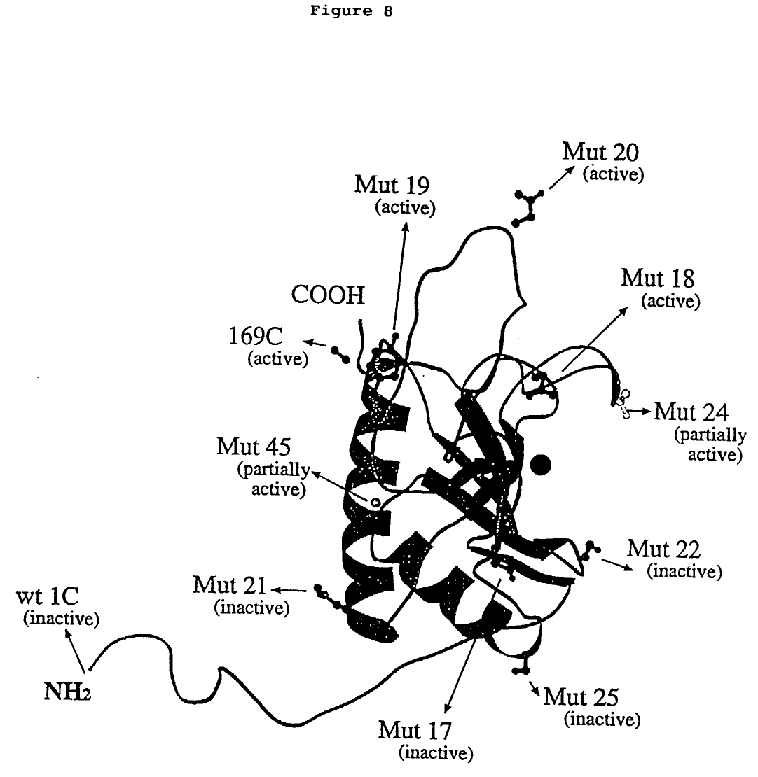

FIG. 8 summarizes the structure-activity data for the 5K PEG modified forms of Shh with respect to the positions of the modified amino acids in the crystal structure of murine Shh. The data reveal that one end of the protein is not necessary for activity and can be pegylated, while the other end of the protein is sensitive to pegylation. Based on these findings it would be straightforward for one skilled in the art and using the processes we described herein, to select other surface exposed amino acids from the region of Shh that is not needed for function, engineer cysteines at these sites, and then express, purify, and pegylate the resulting products.

In addition to defining useful regions for modification the structure-activity analysis also defined regions in the protein that could not be modified without loss of function. To expand upon our understanding of these sites and the impact of modifications at these sites on function we subjected each of the mutants to modification with a series of smaller thiol reactive compounds and asked which of these functional groups affected activity. The series of modification reagents tested were selected to represent different sizes and shapes of the modifying groups and we envisioned that based on this analysis we would be able to determine more precisely if the sites were different in their proximity to the Shh receptor molecules. Results from these analyses with N-ethyl maleimide (Pierce), β-(4-Hydroxyphenyl) ethylmaleimide (Pierce), N-(1-Naphthyl) maleimide (Aldrich), N-(1-Pyrenyl) maleimide (Sigma), 5 K PEG maleimide (Fluka), and 20 K PEG maleimide (Shearwater Polymers, Inc.) are shown in Table II.

TABLE II
Shh-II 192C Double Cys Mutants- Alkaline Phosphatase Induction
Hedgehog proteins were diluted to 2-3 mg Shh/mL with MES buffer pH 6.5 to a final
concentration of 50 mM MES and treated with a slight molar excess of each thiol
reactive compound over free thiol groups in the reaction mixtures for 2 h at room
temperature. Samples were then treated with 0.5 mM DTT for 1 h at room temperature
to quench excess modification reagent and analyzed for activity in the C3H10T1/2
assay.

| Protein | Potency (IC₅₀) in C3H10T1/2 assay in μg/mL | | | | | | |
|---|---|---|---|---|---|---|---|
| Shh variant | unmodified | NEM | HPE | Napth | Pyrene | 5 K PEG | 20 K PEG |
| wild type | 2 | 1 | 0.3 | 0.3 | 0.1 | >20 | >20 |
| C1II/169C | 0.3 | 0.2 | 0.2 | 0.2 | 0.2 | 0.3 | 0.3 |
| mutant 17 +N27C | 0.4 | 0.3 | 0.5 | 1.5 | 3 | 4 | 8 |
| mutant 18 +N46C | 0.4 | 0.2 | 0.4 | 0.4 | 0.5 | 0.3 | 0.3 |
| mutant 19 +Y57C | 0.2 | 0.2 | 0.3 | 0.2 | 0.3 | 0.2 | 0.3 |
| mutant 20 +N68C | 0.3 | 0.2 | 0.2 | 0.3 | 0.3 | 0.2 | 0.4 |
| mutant 21 +N92C | 1.5 | 2 | 2 | 4 | 3 | >20 | >20 |
| mutant 22 +S154C | 1 | 2 | 2 | 2 | 3 | 5 | 8 |
| mutant 23 +K82C | 0.2 | modifications failed | | | | | |
| mutant 24 +S112C | 0.4 | 0.2 | 0.2 | 0.3 | 0.25 | 0.6 | 2 |
| mutant 25 +S133C | 0.4 | 0.2 | 0.2 | 0.6 | 3 | 2 | 2 |

The data clearly indicate that that the six sites where modification with 5K PEG blocked function (C1, N50, N115, S177, S135, and S156) were not equivalent and that two in particular, N50 and S156, were sufficiently close in proximity to the receptor that they also were blocked by modification with the pyrene moiety. The activity of mutant 17 was partially blocked by the hydroxyphenyl moiety, while the activity of mutant 25 was not affected by the hydroxyphenyl moiety. Mutant 25, in contrast, was partially blocked by addition of the napthyl group. One significant advantage of this type of analysis over conventional mutagenesis studies is that one can map the proximity of a specific group on a ligand with its receptor even if the residue is not in specific contact with the receptor by determining the size of a modification reagent needed to block binding. While any of a large number of groups can be used, we have demonstrated the principle with the six groups listed in Table II. An extensive list of commercially available maleimide derivatives is available from Adrich-Sigma, while others can be synthesized using bifunctional cross-linking reagents (see for example those provided by Pierce). This method should also be applicative to other thiol-specific reactive reagents other than maleimides such as haloacetate and vinylsulfone derivatives. The three Shh variants that retained function (mutants 18, 19, and 20) were next tested for PK in rats. All three showed a further increase in half life following IV administration. The elimination half life increased from 3 h for ShhII 192C with a single 20K PEG to 8 h for the three double mutants containing two 20K PEGS and the area under the curve increased from 34 μg/mL h for pegylated ShhII 192C to over 200 for the three double mutants. Mutant 20 showed a slightly longer half life than mutant 18 and 19 and was selected for further study in animals. In patched LacZ mice mutant 20 PEG was as efficacious as Shh II 169C at inducing Beta-gal activity and treatment of mice with mutant 20 resulted in weight gain in the Shh treated animals, a pharmacodynamic measure of hedgehog activity in mice (data not presented here). Pegylated mutant 20 was also assayed for activity in a nerve crush model in mice where the length of time that the animals take to recover from nerve damage was used as a measure of activity. Pegylated mutant 20 was more potent than pegylated Shh II 169C in this model consistent with its longer half life (data not presented here).

Because mutants 18, 19 and 20 all retained function, we generated one construct that contained two of the three mutations yielding three sites for modification and one that contained all four sites for modification. For these mutants, the C1II/Y57C/A169C pEAG1087 template was used with previously described oligonucleotides: (1) N46C: 5′ GAG TTC CTT AAA TCG CTC GGA GCA CCT GGA GAT CTT CCC TTC 3′, which introduces a new Bsp1286I site, and (2) N46C/N68C: oligonucleotide for N46C above and for N68C: 5′ CAT CAG CCT GTC CGC TCC GGT ACA TTC TTC ATC CTT AAA TAT GAT GTC 3′, which introduces a new RsaI site. The fidelity of the resulting constructs was verified by DNA sequencing. Once sequence confirmation was obtained for the NcoI-XhoI fragments from the resulting mutagenized plasmids, the NcoI-XhoI fragments were subcloned into the XhoI-NcoI vector backbone fragment from p6h-SHH to make the E. coli expression vector. Expression vectors were generated by subcloning the DNA inserts into phosphatase-treated, 5.64 kb XhoI-NcoI pET11d vector backbone of p6H-SHH. The presence of the introduced restriction site was reconfirmed in the expression vector. The expression vectors were transformed into competent BL21(DE3)pLysS cells, colonies selected, induced, and screened for SHH expression as previously described. The Shh mutants were purified following the same strategy disclosed in Example 2 for purification of ShhII 169C and the resulting proteins were pegylated also as described.

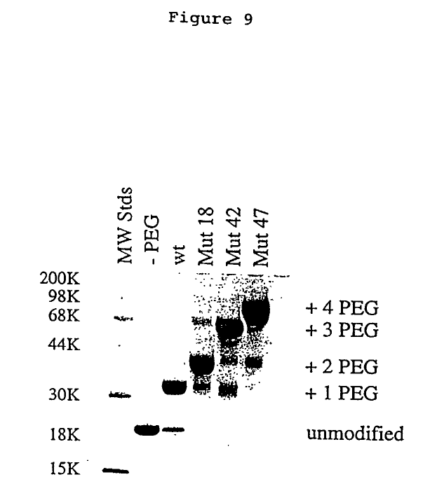

When analyzed by SDS-PAGE we observed that both constructs were pegylated at the newly engineered sites (FIG. 9). The proteins were next tested for function in the C3H10T1/2 assay. The IC50 for mutant 42 that was pegylated with three 5 K PEGs was 1 μg/mL and the IC50 for mutant 47 was approximately 2 μg/mL. The positive data for mutants 42 and 47 in the C3H10T1/2 assay verify that is is possible to put at least 4 PEGs on Shh and retain function. Based on the structure-activity data described here we envision that other hedgehog variants can be made that when pegylated will also retain function and show improved pharmacokinetic/pharmacodynamic activity.

In our initial studies in animals where Shh was administered repeatedly over several weeks or more of treatment, we determined that Shh II 169C that had been pegylated with 20 K maleimide PEG was highly immunogenic and that the antibody response resulted in clearance of the pegylated Shh from the serum. Upon repeat administration of PEG Shh, reduction of Shh levels was frequently observed after only 2 weeks of treatment. As a result of this observation, we devised a simple immunogenicity assay where mice were treated with PEG Shh 3×/week for 3 weeks and at the end of the treatment cycle, serum from the animals was assayed for anti-hedgehog antibodies and levels of Shh in the serum the day after the last Shh treatment. Maleimide and vinylsulfone PEG modified forms of Shh II 169C and mutant 20 Shh were evaluated in this assay and in both sets of analyses the PEG maleimide modified Shh was more immunogenic that PEG vinylsulfone modified Shh. While it may be possible to design a less immunogenic PEG maleimide Shh product, the vinylsulfone adduct was selected for more extensive testing in animals. Mutant 20 that was pegylated with a 20 K vinylsulfone PEG exhibited a similar half life as the corresponding maleimide version, was less immunogenic, and was efficacious in the nerve crush model. Mutant 20 Peg vinylsulfone will also be tested in models for diabetic neuropathy using models for which we have already determined are responsive to Shh.

While the studies described herein largely focus on modifications of Shh and Shh mutants with maleimde and vinsulfonePEG adducts we envision that other types of PEG moieties can be attached at these sites by any chemistry that can be selectively targeted to thiol groups and that PEGs of different sizes and shapes than those described can be used to better maximize the dosing/bioavailability of the PEGylated products.

What is claimed is:

1. A composition comprising a hedgehog protein coupled to a non-naturally-occurring polymer at a position on the hedgehog protein, wherein said polymer comprises a polyalkylene glycol moiety.

2. The composition of claim 1, wherein the at least one polyalkylene moiety is coupled to the hedgehog protein by way of a group selected from a thiol group, an aldehyde group, a maleimide group, a vinylsulfone group, a succinimidyl group, a haloacetate group, plurality of histidine residues, a hydrazine group and an amino group.

3. The composition of claim 2, wherein the group is a thiol group.

4. The composition of claim 2, wherein the group is a maleimide group

5. The composition of claim 2, wherein the group is a vinylsulfone group

6. The composition of claim 2, wherein the group is a haloacetate group

7. The composition of claim 2, wherein the group is a plurality of histidine residues

8. The composition of claim 2, wherein the coupling group is a hydrazine group

9. The composition of claim 2, wherein the group is an amino group.

10. The composition of claim 2, wherein the group is a succinimidyl group.

11. The composition of claim 1, wherein the hedgehog protein is coupled to more than one polymer at more than one position.

12. The composition of claim 1, wherein the hedgehog protein is selected from the group consisting of Sonic hedgehog, Indian hedgehog and Desert hedgehog.

13. The composition of claim 12, wherein the hedgehog protein is a mutant hedgehog protein.

14. The composition of claim 13, wherein the mutant hedgehog protein is any hedgehog protein whose N-terminal cysteine has been modified.

15. The composition of claim 13, wherein the mutant hedgehog protein is selected from the group consisting of: a Sonic hedgehog protein whose Alanine residue number 169 has been replaced with another amino acid residue; a Sonic hedgehog protein whose Glycine residue number 175 has been replaced with another amino acid residue; and a Desert hedgehog protein whose Alanine residue number 170 has been replaced with another amino acid residue.

16. The composition of claim 12, wherein the hedgehog protein is modified at an N-terminal cysteine thereof with a hydrophobic group.

17. The composition; of claim 1, wherein the hedgehog protein is a hedgehog antagonist.

18. The composition of claim 17, wherein the hedgehog antagonist is an antibody homolog.

19. The composition of claim 1, wherein the hedgehog protein retains the potency of hedgehog protein lacking said polymer, as measured in a CH310T1/2 assay.

20. The composition of claim 1, wherein the hedgehog is a hedgehog fusion protein.

21. The composition of claim 20, wherein the hedgehog fusion protein comprises a portion of an immunoglobulin molecule.

22. The composition of claim 1, wherein the hedgehog protein has at least one of the following properties: (a) it is an antagonist of hedgehog activity; (b) it is hydrophobically modified.

23. A composition comprising a biologically active hedgehog protein having a structure exemplified by the formula A—[Sp]—B—[Sp]—X, wherein A is a polyalkylene glycol polymer moiety conjugated to hedgehog protein B via an optional spacer [Sp]; and wherein X is an optional hydrophobic moiety linked to B.

24. The composition of claim 23, wherein A is not conjugated to an N-terminal cysteine or to any lysine of B.

25. A biologically active hedgehog protein having an N- terminal end and a C-terminal end, the hedghog linked to a polymer comprising a polyalkylene glycol moiety, wherein the linkage is at a position other than at the N-terminal end of hedgehog and other than at any lysine of hedgehog.

26. The hedgehog protein of claim 23 or 25, wherein the hedgehog and the polyalkylene glycol moiety are arranged such that the hedgehog protein has an enhanced bioavailability relative to hedgehog protein lacking the polyalkylene moiety.

27. The hedgehog protein of claim 26, wherein the hedgehog protein is selected from the group consisting of Sonic hedgehog, Indian hedgehog and Desert hedgehog.

28. The hedgehog protein of claim 26, wherein the hedgehog protein is modified at an N-terminal cysteine thereof with a hydrophobic group.

29. The hedgehog protein of claim 28, wherein the hydrophobic group comprises at least one isoleucine amino acid residue.

30. The hedgehog protein of claim 26, wherein the hedgehog protein is coupled to the polymer at or near the C- terminal end of the hedgehog.

31. The hedgehog protein of claim 26, wherein the hedgehog protein is coupled to the polymer at a site other than at or near the C- terminal end thereof.

32. The hedgehog protein of claim 26, wherein the hedgehog protein comprises a hedgehog fusion protein.

33. The hedgehog protein of claim 32, wherein the hedgehog fusion protein includes a portion of an immunoglobulin molecule.

34. The hedgehog protein of claim 26, wherein the hedgehog protein has at least one of the following properties: (a) it is a functional antagonist of hedgehog activity; (b) it is modified with a hydrophobic group

35. A conjugated hedgehog protein comprising a hedgehog protein coupled to a polyethylene glycol moiety, wherein the hedgehog is coupled to the polyethylene glycol moiety by a labile bond, wherein the labile bond is cleavable by biochemical hydrolysis and/or proteolysis.

36. A composition according to claims 1 or 23, wherein the polymer has a molecular weight of from about 5 to about 40 kilodaltons.

37. A pharmaceutical composition comprising the hedgehog composition of claim.

38. A method of treating a potential or developed condition or disease state in a mammialian subject with a hedgehog protein, comprising administering to the subject an effective amount of a hedgehog composition comprising said hedgehog coupled to a polyethylene glycol moiety.

39. The method of claim 38, wherein the hedgehog is coupled to the polymer at a site on the hedgehog that is at or near a C-terminal end.

40. The method of claim 38, wherein the hedgehog is coupled to the polymer at a site on the hedgehog that is other than at or near the N-terminal end.

41. The method of claim 38, wherein the hedgehog is coupled to the polymer at a site on the hedgehog that is other than at or near the C-terminal end

42. The method of claim 38, wherein the hedgehog has at least one of the following properties: (a) it is a functional antagonist of hedgehog activity; (b) it is modified with hydrophobic moiety.

43. A method of prolonging the availability of hedgehog protein in an in vivo or in vitro system, comprising coupling said hedgehog to a non-naturally-occurring polymer moiety to yield a coupled polymer-hedgehog composition, and introducing the coupled polymer-hedgehog composition to the in vivo or in vitro system.

44. The method of claim 43, wherein the hedgehog is coupled to the polymer at a site on the hedgehog protein that at or near a C-terminal end of the hedgehog.

45. The method of claim 43, wherein the hedgehog is coupled to the polymer at a site on the hedgehog that is other than at or near an N-terminal end of the hedgehog.

46. The method of claim 43, wherein the hedgehog is coupled to the polymer at a site on the hedgehog that is other than at or near a C-terminal end of the hedgehog.

47. The method of claim 43, wherein the polymer comprises a polyalkylene glycol.

48. The method of claim 43, wherein the introduced coupled-polymer hedgehog composition comprises the composition of claim 23.

49. The method of claim 47, wherein the coupled-polymer hedgehog composition is produced by coupling a polyalkylene glycol to hedgehog that is immobilized.

50. The method of claim 49, wherein hedghog is immobilized on a resin.

51. The method of claim 50, wherein the resin is a cation exchange resin.

52. A composition comprising a hedgehog protein coupled to a non-naturally-occurring polymer at a position on the hedgehog protein, wherein said polymer is selected from the group consisting of dextrans, polyvinyl pyrrolidones, glycopeptides, and polyamino acids.

53. A composition comprising a hedgehog protein coupled to a non-naturally-occurring polymer at a position on the hedgehog protein, wherein said polymer comprises a heteropolymer, one of whose components is a polyalkylene glycol moiety.

54. A method for defining a functionally important region of a protein, comprising the steps of: identifying amino acid residues of the protein whose side chains are accessible to modification with a modification reagent; optionally altering such accessible residues to allow modification with the modification reagent, modifying such accessible residues independently or in combination with the modification reagent to form a modified protein; testing the modified protein for function; and determining from structure-activity data of the modified protein positions of the identified amino acid residues that affect activity.

55. A method for mapping a functionally important region of a molecule, comprising the steps of modifying said molecule at one or more specific sites on the molecule with a series of modification reagents of different molecular size and shape; determining activity of the molecule when the molecule has been so modified with the series of modification reagents; and determining at which size and shape such modification blocks activity of the molecule.

56. The method of claim 55, wherein the step of modifying said molecule comprises modifying said molecule with a polyalkylene glycol moiety.
